(19) United States
(12) Patent Application Publication
(10) Pub. No.: US 2017/0051282 A1
(43) Pub. Date: Feb. 23, 2017

(54) EXTRACELLULAR VESICLE METHODS AND COMPOSITIONS

(71) Applicant: Cold Spring Harbor Laboratory, Cold Spring Harbor, NY (US)

(72) Inventors: Thomas R. GINGERAS, Cold Spring Harbor, NY (US); Sudipto K. CHAKRABORTTY, Cold Spring Harbor, NY (US); Ashwin PRAKASH, Cold Spring Harbor, NY (US); Gal NECHOOSHTAN, Cold Spring Harbor, NY (US)

(21) Appl. No.: 15/217,455

(22) Filed: Jul. 22, 2016

Related U.S. Application Data
(60) Provisional application No. 62/195,953, filed on Jul. 23, 2015.

(51) Int. Cl. C12N 15/113; C12Q 1/68

(52) U.S. Cl. CPC C12N 15/113; C12Q 1/6886; C12Q 2600/178; C12Q 2600/136; C12N 2330/50; C12N 2320/30; C12N 2310/11

(57) ABSTRACT

Disclosed herein are methods and compositions for treating cancers.

### CROSS-REFERENCE

This application claims the benefit of U.S. Provisional Patent Application No. 62/195,953, filed on Jul. 23, 2015 which is herein incorporated by reference in their entirety.

### STATEMENT OF GOVERNMENTAL SUPPORT

This invention was made with government support under 1U54HG007004 and CA045508 awarded by the National Human Genome Research Institute. The government has certain rights in the invention.

### SEQUENCE LISTING

The instant application contains a Sequence Listing which has been submitted electronically in ASCII format and is hereby incorporated by reference in its entirety. Said ASCII copy, created on Sep. 7, 2016, is named 48562-701_201_SL.txt and is 11,237 bytes in size.

### BACKGROUND

Historically, microvesicles were regarded as cellular debris with no apparent function. However, a growing body of experimental data has suggested that microvesicles have numerous biological activities. For example, platelet-derived microvesicles were shown to stimulate selected cells via surface proteins on the microvesicles. In other examples, specific effects of bioactive lipids in platelet microvesicles on certain target cells were identified. In still further examples, platelet microvesicles increased adhesion of mobilized CD34⁺ endothelial cells by transfer of certain microvesicle surface components to the mobilized cells. Microvesicles also play a crucial role in disseminating pathogens such as prions and viruses from one cell to another. Single and double-stranded RNAs can also be pathogen-associated molecular signals that are recognized by cytosolic receptors of the innate-immune system of many cell types during virus infection. This recognition of exogenous RNAs can result in the activation of caspase-1 and subsequent apoptosis of affected cells. Differentiation of endogenous from exogenous RNAs is partially based on the presence of 5′ triphosphate or poly-uracil or -adenylyl strings frequently found in RNA viral genomes.

Microvesicles also comprise RNA that may reflect the RNA content of the cell from which they originate. Microvesicles have biological effects on other cells, probably due to the RNA present in the microvesicles. Microvesicles reportedly include non-coding miRNA (microRNA) that could potentially interfere or regulate gene expression in cells that produced the microvesicles. In vitro cell-to-cell signaling via exosomal RNA has also been demonstrated. Some exosomal RNA was functional and translatable in a recipient cell; however, many exosomal RNAs were not present in the cytoplasm of cells from which the exosomes were thought to have originated. While RNA was generally instable in serum and readily hydrolyzed by RNAses, other RNA was resistant to RNAse attack, presumably due to its varying association with circulating particles. Chemically and structurally the RNA associated particles are reportedly diverse. The literature contains a number of contradictions. Several papers, for example, indicate that the RNA “cargo” of exosomes was substantially different from the parental cell content. This runs counter to several other reports noting that the miRNA content for their originating cancer cells was similar to that found in circulating exosomes. Complicating factors between these studies include a lack of standardized techniques, protocols, and workflows for isolation of exosomes and downstream analysis of their constituents. Thus, contradictory data and hypotheses with respect to the nature, quality, availability, and origin/manner of generation of microvesicles exist.

### SUMMARY

In one aspect a composition is provided comprising an antisense masking oligonucleotide (AMO), wherein the AMO has anti-tumor activity, specifically binds to a RNA fragment of a primary RNA transcript of an extracellular cancer vesicle (ECV) and inhibits tumor progression mediated by the RNA fragment.

In some embodiments, the AMO binds to a RNA fragment that is a 5′ RNA fragment. In some embodiments, the AMO binds to a RNA fragment that is an external loop cleavage product of the primary RNA transcript. In some embodiments, the AMO binds to a RNA fragment that is an internal loop cleavage product of the primary RNA transcript. In some embodiments, the AMO binds to a region of the RNA fragment that is duplexed in the primary RNA transcript. In some embodiments, the AMO does not interact with the primary RNA transcript. In some embodiments, the AMO binds to a RNA fragment comprises a single stranded region. In some embodiments, the AMO binds to a single stranded portion of a RNA fragment. In some embodiments, the AMO does not bind to a RNA fragment that is double stranded. In some embodiments, the AMO does not bind to a duplexed region of a RNA fragment.

In some embodiments, the AMO binds to a RNA that is a human (h)Y RNA. In some embodiments, the AMO binds to a RNA that is not a RNY1 RNA, a RNY3 RNA, a RNY4 RNA, or a combination thereof. In some embodiments, the AMO binds to a RNA is a hY5 RNA.

In some embodiments, the AMO binds to an RNA that has a primary RNA transcript that is transcribed by RNA polymerase III.

In some embodiments, the AMO binds to a RNA fragment that is from about 8 to 40 nucleotides in length. In some embodiments, the AMO binds to a RNA fragment that is from about 8 to 31 nucleotides in length. In some embodiments, the AMO binds to a RNA fragment that is from about 23 to 40 nucleotides in length. In some embodiments, the AMO binds to a RNA fragment that is from about 23 to 31 nucleotides in length. In some embodiments, AMO binds to a RNA fragment that is about 23, 29, or 31 nucleotides in length.

In some embodiments, the AMO binds to a RNA fragment that is processed from a primary RNA transcript in the ECV. In some embodiments, the AMO binds to a RNA fragment that is cleaved from a primary RNA transcript in the ECV. The In some embodiments, the AMO does not bind to a primary RNA transcript that is processed in the ECV to form the RNA fragment. The In some embodiments, AMO does not bind to a primary RNA transcript that is cleaved in the ECV to form the RNA fragment.

In some embodiments, the AMO binds to a RNA fragment that comprises a secondary structure. In some embodiments, the secondary structure of the RNA fragment is a hairpin.

In some embodiments, the AMO binds to a RNA fragment that comprises the sequence 5′ GUU GUG GG 3′ (SEQ ID NO: 1). In some embodiments, the AMO binds to a sequence 5′GUU GUG GG 3′ (SEQ ID NO: 1) of the RNA fragment that is not duplexed. In some embodiments, the AMO does not bind to a 5′GUU GUG GG 3′ (SEQ ID NO: 1) of the primary RNA transcript that is duplexed.

In some embodiments, the AMO binds to a RNA fragment that is in the ECV.

In some embodiments, the AMO does not bind to a primary RNA transcript that is in the ECV. In some embodiments, the AMO does not bind to a primary RNA transcript that is in a cancer cell. In some embodiments, the AMO does not bind a primary RNA transcript that is in a normal cell. In some embodiments, the AMO does not bind to a RNA fragment that is in an extracellular vesicle from a normal cell.

In some embodiments, the AMO binds to a RNA fragment that does not comprise a 5′ triphosphate, a 5′ poly-uracil string, or a 5′ polyadenylyl string.

In some embodiments, the AMO binds to a RNA fragment of the ECV that has a diameter of from 30 nm to 2 μm.

In some embodiments, the AMO binds to a RNA fragment of an ECV that is an exosome. In some embodiments, the AMO binds to a RNA fragment of an ECV that is a microvesicle. In some embodiments, the AMO binds to a RNA fragment of an ECV that is not an apoptotic body. In some embodiments, the AMO binds to a RNA fragment of an ECV that is not formed by blebbing.

In some embodiments, the AMO binds to a RNA fragment of an ECV that comprises programmed cell death 6-interacting protein (PDCDIP). In some embodiments, the AMO binds to a RNA fragment of an ECV that comprises transferrin receptor (CD71). In some embodiments, the AMO binds to a RNA fragment of an ECV that comprises TSG101. In some embodiments, the AMO binds to a RNA fragment of an ECV that comprises an Endosomal Sorting Complexes Required for Transport (ESCRT) protein complex In some embodiments, the AMO binds to a RNA fragment of an ECV that does not comprise rRNA 2′-O-methyltransferase fibrillarin protein. In some embodiments, the AMO binds to a RNA fragment of an ECV that does not comprise prohibitin (PHB) protein. In some embodiments, the AMO binds to a RNA fragment of an ECV that does not comprise protein disulfide isomerase (PDI) protein.

In some embodiments, the AMO binds to a RNA fragment of an ECV that localizes to the cytoplasm of a normal cell when the ECV contacts the normal cell.

In some embodiments, the AMO localizes into the ECV. In some embodiments, the AMO is single stranded.

In some embodiments, the AMO comprises RNA.

In some embodiments, the AMO is chemically modified.

In some embodiments, the AMO is resistant to degradation when administered to a mammal.

In some embodiments, the AMO is not expressed from an expression vector.

In some embodiments, the AMO comprises the sequence 5′-CCC ACA AC-3′ (SEQ ID NO: 7).

In some embodiments, the AMO comprises a backbone modification. In some embodiments, the AMO comprises a phosphorothioate linkage or a phosphorodiamidate linkage. In some embodiments, the AMO comprises at least one modified sugar moiety. In some embodiments, each sugar moiety is a modified sugar moiety. In some embodiments, the AMO comprises a phosphorodiamidate morpholino (PMO), a locked nucleic acid (LNA), a peptide nucleic acid (PNA), a 2′-O-methyl (2′-O-Me), a 2′-Fluoro (2′F), or a 2′-O-methoxyethyl (2′MOE) moiety.

In some embodiments, the AMO inhibits apoptosis of non-tumor cells in a tumor microenvironment. In some embodiments, the AMO inhibits angiogenesis in a tumor microenvironment. In some embodiments, the AMO inhibits metastasis. In some embodiments, the AMO inhibits inflammation. In some embodiments, the AMO inhibits cell migration.

In one aspect, a pharmaceutical composition is provided comprising any composition described herein.

In one aspect, a composition or pharmaceutical composition provided herein is for use in the treatment of cancer.

In one aspect, a composition or pharmaceutical composition provided herein is for use in the manufacture of a medicament for treating cancer.

In one aspect, an isolated ECV is provided herein comprising an AMO that specifically binds to a RNA fragment of a primary RNA transcript of the ECV, wherein the RNA fragment mediates tumor progression.

In one aspect, a method of producing a therapeutic ECV is provided comprising an antisense masking oligonucleotide (AMO) with anti-tumor activity that specifically binds to a RNA fragment of a primary RNA transcript of the ECV, wherein the RNA fragment mediates tumor progression, comprising: providing a cancer cell that can produce ECVs; allowing the cancer cell to produce the ECVs; transfecting an AMO in the ECVs; and isolating exosomes produced by the cell, wherein the ECVs comprise the AMO bound to the RNA fragment of a primary RNA transcript.

In one aspect, a method of producing a therapeutic ECV is provided comprising the steps: isolating ECVs from a biological sample from a subject, wherein the ECVs comprise a RNA fragment of a primary RNA transcript; contacting the ECVs with an antisense masking oligonucleotide (AMO) with anti-tumor activity that inhibits tumor progression mediated by the RNA fragment, to thereby produce therapeutic extracellular vesicles.

In one aspect, a method of producing a therapeutic ECV is provided comprising the steps: isolating donor cells from a biological sample from a subject; isolating extracellular vesicles produced by the donor cells, wherein the extracellular vesicles comprise a RNA fragment of a primary RNA transcript; and contacting the extracellular vesicles with an AMO with anti-tumor activity, thereby producing therapeutic extracellular vesicles.

In one aspect, a method of identifying an AMO that inhibits tumor progression mediated by a RNA fragment of a primary RNA transcript of an ECV is provided, comprising: providing a testing system comprising ECVs and target cells, wherein the ECVs are located in proximity to the target cells; measuring tumor progression of the target cells; and identifying an AMO with anti-tumor activity that inhibits tumor progression mediated by a RNA fragment of a primary RNA transcript of the ECVs.

In some embodiments, the system further comprises a cancer cell population that produces the ECVs.

In one aspect, an in vitro cell culture system is provided comprising a cancer cell population that produces ECVs comprising a RNA fragment of a primary RNA transcript; a target cell population; and an antisense masking oligonucleotide (AMO) with anti-tumor activity that inhibits tumor progression mediated by the RNA fragment.

In some embodiments, the target cell population is a normal cell population.

In one aspect, a kit is provided comprising an antisense masking oligonucleotide (AMO) with anti-tumor activity that specifically binds to a RNA fragment of a primary RNA transcript of an ECV; and a detecting reagent or a detecting apparatus capable of detecting binding of the AMO to the RNA fragment, wherein the RNA fragment mediates tumor progression.

In one aspect, a method of treating cancer in a mammal is provided comprising administering to the mammal a pharmaceutical composition comprising any composition described herein.

In one aspect, a method of treating cancer in a subject is provided, comprising administering an effective amount of an isolated ECV comprising an AMO with anti-tumor activity that specifically binds to a RNA fragment of a primary RNA transcript of the ECV, wherein the RNA fragment mediates tumor progression.

In one aspect, a method of treating cancer in a mammal is provided comprising administering to the mammal a pharmaceutical composition comprising an antisense masking oligonucleotide (AMO) with anti-tumor activity that specifically binds to a RNA fragment of a primary RNA transcript of an ECV, wherein the RNA fragment mediates tumor progression.

In some embodiments, administering comprises administering locally to a tumor microenvironment.

In one aspect, a method of inhibiting tumor cell progression in a tumor microenvironment is provided comprising contacting an ECV in the tumor microenvironment with a composition comprising an AMO with anti-tumor activity that specifically binds to a RNA fragment of a primary RNA transcript of the ECV, wherein the RNA fragment mediates tumor progression.

In some embodiments, the ECV is not a circulating vesicle.

In some embodiments, apoptosis of non-tumor cells in the tumor microenvironment is inhibited. In some embodiments, angiogenesis is inhibited in the tumor microenvironment In some embodiments, metastasis is inhibited. In some embodiments, inflammation is inhibited. In some embodiments, cell migration is inhibited.

In some embodiments, the method further comprises administering an anti-cancer agent.

In some embodiments, stromal cell death is inhibited. In some embodiments, epithelial cell death is inhibited. In some embodiments, endothelial cell death is inhibited. In some embodiments, fibroblast cell death is inhibited.

In some embodiments, the AMO localizes to within the ECV in the microenvironment after the administering.

In some embodiments, the composition comprises an ECV internalizing agent.

In one aspect, a method of inhibiting metastatic disease progression in a subject is provided comprising: selecting a subject having an ECV comprising a RNA fragment of a primary RNA transcript, wherein the RNA fragment mediates tumor progression; and administering, to the selected subject, an AMO with anti-tumor activity, wherein the AMO specifically binds to the RNA fragment under conditions effective to inhibit progression of metastatic disease in the subject.

In one aspect, a method of inhibiting pre-metastatic site formation in a subject is provided comprising: selecting a subject having an ECV comprising a RNA fragment of a primary RNA transcript, wherein the RNA fragment mediates tumor progression; and administering, to the selected subject, an AMO with anti-tumor activity, wherein the AMO specifically binds to the RNA fragment under conditions effective to inhibit formation of a pre-metastatic site in the subject.

In one aspect, a method of inhibiting primary tumor growth in a subject is provided comprising: selecting a subject having an ECV comprising a RNA fragment of a primary RNA transcript, wherein the RNA fragment mediates tumor progression; and administering, to the selected subject, an AMO with anti-tumor activity, wherein the AMO specifically binds to the RNA fragment under conditions effective to inhibit growth of a primary tumor in the subject.

In one aspect, a method of diagnosing a mammal with cancer is provided comprising: isolating ECVs from a biological sample from a mammal; and detecting the presence of a RNA fragment of a primary RNA transcript of the ECV, wherein the RNA fragment mediates tumor progression, wherein the presence of the RNA fragment in the biological sample indicates that the subject has cancer.

In some embodiments, the isolated ECVs comprise circulating ECVs.

In some embodiments, the isolated ECVs comprise ECVs from a tumor microenvironment.

In some embodiments, the cancer is carcinoma, melanoma, lymphoma, leukemia, neuroblastoma, retinoblastoma, glioma, rhabdomyoblastoma, or sarcoma.

In one aspect, a method for evaluating treatment efficacy and/or progression of a cancer in a subject is provided, comprising: isolating ECVs from a biological sample of a subject; determining an amount of a RNA fragment or amount of tumor progression mediated by the RNA fragment, wherein the RNA fragment is a fragment from a primary RNA transcript in the ECVs; and determining any measurable change in the amount or of the pro-RNA fragment or amount of tumor progression to thereby evaluate treatment efficacy and/or progression of the cancer in the subject.

In one aspect, a method of monitoring metastatic disease treatment in a subject is provided comprising: obtaining first and second samples, at different points in time, from a subject being treated for a metastatic disease; measuring an amount of a RNA fragment or amount of tumor progression mediated by the RNA fragment in ECVs in each sample, wherein the RNA fragment is a fragment of a primary RNA transcript; comparing the amount of the RNA fragment or amount of tumor progression in the first sample to a corresponding level in the second sample; and determining whether the subject is responding to a treatment based on the comparing.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIGS. 1A-D exemplify validation of purification of extracellular vesicles (EVs). (A) Transmission electron microscopy image of K562 EVs after negative staining shows classic cup-shaped vesicles that are on average smaller than 200 nm. (B) Immuno-electron microscopy image of purified EVs labeled with Anti-CD81 (mouse mAb) and detected by Goat anti-mouse IgG secondary conjugated with 5 nm gold. Dark spots on the image are the electron dense gold elements conjugate to IgG secondary antibody. (C) Bioanalyzer RNA profile (RNA Pico-chip) of untreated EVs, RNA profile of EVs treated with RNAse and RNA profile of EVs treated with detergent and RNAse. X-axis was nucleotides lengths and Y-axis was Fluorescent Units. (D) Western Blot analysis of proteins from K562 EVs and whole cell. Proteins selected for detection were previously identified to be enriched in EV or whole cell. EV enriched: ALIX (PDCD6IP gene), CD71 (TfR1 gene), TSG101 (TSG101 gene). Whole cell: PDI (PDI gene), FIBRILLARIN (FBL gene), PROHIBITIN (PHB gene).

FIGS. 2A-D exemplify pie-charts representing the relative abundance of families of RNA within BJ whole cell (A), K562 whole cell (B), BJ EV (C), and K562 EV (D). The group labeled as “Others” in the pie-charts are representative of reads derived from several gencode annotation categories such as pseudogenes, antisense intronic, mitochondrial t-RNA, vault RNA, immunoglobulin genes etc.

FIGS. 3A-F exemplify fragmentation patterns of hY5 (A) Full length hY5 structure (SEQ ID NO: 6). The structure was drawn using mfold (van Gelder et al. NAR 1994 Vol 22, No. 13 p. 2505). Bold line indicates the 5′ 31nt processed product and the 8nt motif us highlighted. (B) Graph depicting the most frequent (>1000 reads per million) start and stop positions of reads mapping to the human hY5 gene. The most frequent start positions marked as the 5′ start position of the hY5 annotation, and position 52 of the annotation. And the most frequent stop positions being 23, 29, and 31 for the reads which start at the 5′ end of the hY5 gene, and position 83 which has reads starting at 52 and also some reads that start at position 1. (C) Northern blot of hY5 RNA purified from K562 and BJ cells and EVs. Synthetic versions of Y5 processing products were used as size markers. RNA was detected by a probe complementary to the 5′ 31nt processed product. w-whole cell RNA, e-EV RNA. (D, E) In vitro processing of hY5. Synthetic full length hY5 was incubated for 30 min at 37° C. with 0, 2, 4, or 8 μg of K562 whole cell (D) or EV (E) protein extract. Samples containing only the extracts and treated identically were used to control for the existence of Y5 RNA in protein extracts. Detection was done as in C. 23nt and 31nt size markers are not equimolar. (F) In vitro processing of Y5 5′ 31-mer variants. Wild type (WT), scrambled (scram.) and 8nt motif scrambled (motif scram.) versions of the Y5 5′ 31-mer were radioactively end-labeled and incubated with K562 EV protein extract for 2 hr at 37° C.

FIGS. 4A-F exemplify quantification of cell death by Flow Cytometry. YO-PRO-1 and Hoechst dyes were used for quantification of cell death. Y-axis indicates the percent of cell death indicated by YO-PRO-1 and Hoechst double positive cells. The mean of duplicates was presented with error bars indicating variation from mean. (A) Levels of cell death in K562 cells when treated with EVs and EV RNA. Y-axis indicates percent cell death observed. The following treatments are presented: Untreated: K562 cells without any treatment, K562 EV treated: K562 cells incubated with K562 EVs, Mock: K562 cells with lipofectamine treated only (no RNA). K562 EV RNA treated: K562 cells treated with K562 EV RNA. Complete scram 31-mer: K562 cells treated with 31 nucleotide scrambled sequence. (B) Levels of cell death in BJ cells when treated with EVs and EV RNA. Y-axis indicates percent cell death observed. The following treatments are presented: Untreated: BJ cells without any treatment Mock: BJ cells with lipofectamine treated only (no RNA), BJ EV RNA: BJ cells transfected with BJ EV RNA, K562 EV RNA: BJ cells treated with K562 EV RNA, BJ EV: BJ cells incubated with BJ EVs, HeLa EV: BJ cells incubated with HeLa EVs, U2OS EV: BJ cells incubated with U2OS EVs, MCF7 EV: BJ cells incubated with MCF7 EVs, K562 EV: BJ cells incubated with K562 EVs. (C) Generality of hY5 31-mer induced cell death phenotype. Bars indicate the net increase in cell death normalized to levels of cell death from mock treatment in each cell type. Four cancer cell lines including K562 (chronic myelogenous leukemia), HeLa (cervical adenocarcinoma), MCF7 (breast adenocarcinoma), U2OS (Osteosarcoma) and 4 primary cells including BJ (normal skin fibroblasts), HUVEC (normal human umbilical vein endothelial cell), IMR90 (normal human lung fibroblasts) and HFFF (normal human fetal foreskin fibroblasts) were transfected with hY5 31-mer. 100 pmol of hY5 was used for each transfection, except HFFF where 200 pmol of hY5 31-mer was used. (D) Dose response curve of hY5 31-mer induced cell death phenotype in BJ cells. The bars represent the percent of cell death when BJ cells are treated with increasing dose (10, 50, 100, 200, 300 and 400 pmol) of hY5 31-mer or nonspecific RNA. AllStars negative control RNA (Qiagen) was used as a non-specific RNA control. The levels of cell death in Untreated or Mock treated (Lipofectamine only) BJ cells are also indicated. (E) Levels of cell death in BJ cells from 100 pmol of synthetic RNA oligonucleotides transfection. Y-axis indicates the percent cell death. The synthetic RNA oligonucleotides used for transfection are as follows: Untreated: BJ cells without any treatment, Mock: BJ cells with lipofectamine treated only (no RNA), Nonspecific RNA: Nonspecific RNA control (AllStars negative control siRNA), 8nt motif deleted: hY5 sequence with nucleotides 14-21 motif deleted, hY5 31-mer complement: 31nt hY5 3′ side fragment, 8nt motif scrambled: hY5 31-mer sequence with nucleotides 14-21 scrambled, hY5 31-mer scram: 31nt completely scrambled sequence, DS hY5 31-mer, Double stranded hY5 31-mer duplex, Full length hY5: hY5 83-mer full length sequence, hY5 31-mer: 5′ hY5 31nt fragment, hY5 23-mer: 5′ side hY5 23nt fragment. (F) Levels of cell death observed in K562 cells from 100 pmol of synthetic RNA oligonucleotides transfection. Y-axis indicates percent cell death. The synthetic RNA oligonucleotides used for transfection are as follows: Untreated: K562 cells without any treatment, Mock: K562 cells with lipofectamine treated only (no RNA), Nonspecific RNA: Nonspecific RNA control (AllStars negative control siRNA), 8nt motif deleted: hY5 sequence with nucleotides 14-21 motif deleted, 8nt motif scrambled: hY5 31-mer sequence with nucleotides 14-21 scrambled, hY5 31-mer scram: 31nt completely scrambled sequence, DS hY5: Double stranded, Full length hY5 83-mer, hY5 31-mer: 5′ hY5 31nt fragment.

FIG. 5 exemplifies schematic of a protocol for isolation of EVs from conditioned cell culture medium.

FIG. 6 exemplifies a graph of the amount and size distribution of K562 EVs by Nanoparticle Tracking analysis (NTA). X-axis represents particle size (nm). The Y-axis represents the concentration of particles (1×10⁶)/mL.

FIGS. 7A-B exemplify scatter plots representing correlation in gene expression levels, between replicates of EVs and cellular small RNA in K562 (A) or BJ (B).

FIGS. 8A-B exemplify graphs depicting kernel density plots of the ratio of rpm in EV and the sum of rpm in EV and corresponding whole cell in K562 (A) and BJ (B). Each line in the plots depicts the number of genes belonging to each RNA family, and genes which have a ratio of 0 represents genes that are more abundant in cells compared to EVs, which a ratio of 1 represents genes that are more abundant in EVs when compared to their source cells.

FIGS. 9A-D exemplify intercellular transfer and subcellular localization of EVs and EV-RNA. (A) Transfer and subcellular localization of K562 EVs labeled with lipid dye PKH67 in BJ cells. (B) Transfer and subcellular localization of 5-ethynyl uridine (EU) labeled K562 EV RNA (green) in Mouse 3T3 cells treated with ActinomycinD. Nuclei are counterstained with Hoechst. The scale bar represents 20 μm. (C) Subcellular localization of synthetic hY5 31-mer labeled with Alexa-488 at 3′end in BJ cells after 24 hr Scale bar indicates 15 μm. (D) Time course analysis of the level of hY5 31-mer in mouse HB4 cells when Mouse HB4 cells are incubated with K562 EVs. X-axis indicates duration of incubation (hr) while Y-axis indicates the level of hY5 (in reads per million).

FIG. 10 exemplifies quantification of cell death of BJ cells by co-culture with K562. Y-axis indicates the percent cell death: Untreated: BJ cells grown without any treatment, Transwell: Percent cell death observed in BJ cells when co-cultured with K562 cells across a Transwell membrane (1 μm pore size) at 1:1 ratio, Direct co-culture: Percent cell death observed in primary BJ cells when BJ cells are directly co-cultured in the same well with K562 cells at 1:1 ratio.

FIGS. 11A-F exemplify a novel method of exosome isolation and a multi-parametric comparative analysis to other exosome isolation methods. (A) Schematic of exemplary method of exosome isolation. (B) Graph of the amount and size distribution of EVs isolated using the indicated isolation methods. X-axis represents particle size (nm). The Y-axis represents the concentration of particles (1×10⁶)/mL. (C) Scatter plots representing correlation in gene expression levels, between replicates of EVs and cellular small RNA using the indicated isolation methods. (D) Graph comparing RNA yield using the indicated isolation methods. (E) Graph comparing exosome sizes obtained using the indicated isolation methods. (F) Graph comparing number of isolated exosomes using the indicated isolation methods.

### DETAILED DESCRIPTION

Several aspects are described below with reference to example applications for illustration. It should be understood that numerous specific details, relationships, and methods are set forth to provide a full understanding of the features described herein. Those having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### Fragments of Small RNAs in Extracellular Vesicles Shape Microenvironments of Cancer Cells

In the late 19^{th} century, Paget proposed that the microenvironment was key for tumor growth. For metastasis, migratory tumor cells leave the primary tumor through intravasation, disseminate throughout the body via the circulation, and eventually engraft in a distant organ that provides an appropriate microenvironment. The ability of cancer cells to migrate and traverse the epithelial and endothelial barriers in the primary tumor site, and, once disseminated, to invade, survive, and colonize the metastatic site, are prerequisites for metastasis. The tumor microenvironment is a key contributor for cancer progression and drug resistance.

Extracellular vesicles (EVs) contain many proteins and various types of RNAs as cargo. The issue of elucidating the functionality of RNAs released and carried by EVs remains largely unresolved. Determining a functionality of these EV RNAs is complicated, for example, because, for example, a large proportion of the detected RNA biotypes are represented by a mixture of full length and shorter fragments. Furthermore, while tumor derived EVs have enhanced expression of tumor antigens and do not mirror the general protein composition of the plasma membrane of the originating tumor cell, limited information is available regarding RNA content of EVs and the function of only a few of these molecules is partially understood. EVs can communicate with and influence neighboring and distal cells. Cancer-secreted EVs can be internalized by other cell types in a cancer microenvironment site and their contents can be transferred to recipient site cells and exert genome-wide regulation of gene expression. Furthermore, tumor-derived EVs can upregulate proinflammatory molecules at potential metastatic sites. For example, preconditioning of cells or potential metastatic sites with EVs from a number of cancer cell lines can increase the metastatic tumor burden and distribution in target tissues, regardless of their origin or metastatic capability. In addition, RNAs of cancer-derived EVs may activate Toll-like receptors in surrounding immune cells. Therefore, cancer-secreted miRNAs may play a crucial role in regulating various cellular components of the tumor microenvironment in order to facilitate metastasis. The adaptation of primary and metastatic sites by EVs to facilitate cancer cell dissemination and engraftment can play an important pro-metastatic role. The extracellular presence of sRNAs suggests a potential role for sRNAs in defining the metastatic potential of cancer cells and mediating the cancer-host communication.

There is a need to dissect the structure and the function of EVs and their contents and utilize this information to develop minimally invasive diagnostics and therapeutics. Detection of specific RNA and protein molecules in exosomes derived from body fluids can be a minimally invasive way of identifying diagnostic and prognostic biomarkers of various pathological conditions, including cancer. There is a need to develop therapeutics, to develop predictive or early diagnostic markers for metastasis, and to elucidate the molecular mechanisms of metastasis that would allow development of efficient treatment options.

Intercellular communication can be mediated by extracellular small regulatory RNAs (sRNAs). To date, most attention is centered on exosomes and microRNAs as the vectors and the secreted species, respectively. However, this field would benefit from an increased understanding of the plethora of sRNAs secreted by different cell types in different extracellular fractions. It is still not clear if specific sRNAs are selected for secretion, or if sRNA secretion is mostly passive. Various members of the hY RNA families and can be contained in EVs as RNA cargos. However, the relationship between full length primary transcript hY RNAs and processed hY5 forms, and whether these forms are biologically active, had previously remained elusive. Additionally, no differences between the processed and the primary Y RNA transcripts in the EVs released by different types of normal and transformed cells were previously known to exist.

It has now been discovered that a sRNA processed specifically in EVs and released from cancer cells plays an important role in influencing the microenvironment in the competition of normal and cancer cells in vitro and may do so under in vivo conditions. The inventors have also observed that some sRNAs are found in both cancer cell-derived EVs and in non-cancer derived EVs. In some embodiments, shorter fragments of these sRNAs are found in cancer derived EVs and are absent from, or at much lower levels than, non-cancer derived EVs. In some embodiments, shorter fragments of these sRNAs are found in cancer cell-derived EVs and non-cancer cell-derived EVs; however, these shorter fragments within the cancer cell-derived EVs display pro-apoptotic activity, while these shorter fragments within the non-cancer cell-derived EVs do not display pro-apoptotic activity. The inventors have discovered that when human primary cells of multiple types are exposed to EVs from a variety of human cancer cell lines of distinct developmental lineages, rapid cell death of the primary cells occurs. Cancer cells treated with EVs from primary cells or cancer cells do not display, or have less of, this response. For example, cancer cell EVs processes hY5 transcripts into single stranded 31nt and 23nt sRNA products that triggers cell death specifically in primary cells of diverse developmental origins. Furthermore, it has been discovered that sRNAs processed specifically in EVs and released from cancer cells may play an important role conditioning pre-metastatic sites or microenvironments and facilitate seeding of circulating cancer cells at metastatic sites.

The functional role of hY5 fragments orchestrated through extracellular vesicles can be an intricate competitive cell interaction mechanism, and can promote in vivo establishment, growth, and spread of tumor cells. The transfer of cancer cell EVs via cell to cell interactions can also result in primary cell death and contribute to establishment of altered microenvironments that can favor cancer cell development process, e.g., growth, invasion, metastasis. The results suggest an in vivo role for hY5 fragments in a tumor microenvironment. In some embodiments, lethality induced by hY5 fragments can sensitize normal tissue to neoplastic cell invasion and metastasis by promoting cell removal and inducing an inflammatory response. For example, EVs can signal formation of microenvironments that favor cancer cell growth. For example, these microenvironments can favor cancer metastasis. For example, microenvironments created by cancer cell EVs can favor seeding and/or growth of circulating cancer cells at secondary sites, thus potentiating metastasis.

The inventors have unexpectedly discovered that EVs produced from cancer cells can be modified or inhibited and employed to treat cancer and/or prevent cancer progression and/or metastasis. The inventors still further discovered that RNA-containing EVs can be employed in numerous diagnostic applications and represent targets for therapeutics. In some embodiments, RNAs contained in the EVs are modified or inhibited. In some embodiments, nucleic acids of EVs, such as pro-apoptotic, pro-inflammatory, or pro-metastatic nucleic acid fragments contained within EVs produced by cancer cells, are modified or inhibited.

### DEFINITIONS

“About” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

“Antisense masking oligonucleotide” (AMO) refers to a nucleic acid that inhibits a function or an activity of a non-mRNA target polynucleotide. AMOs do not include siRNAs or miRNAs

“Anti-tumor activity” refers to the in vitro and/or in vivo anti-tumor effects exerted by the AMOs according to the invention. Anti-tumor effects include, but are not limited to, a decrease of cell growth, a decrease of a pro-apoptotic effect, an anti-migratory effect, an anti-inflammatory effect, an anti-metastatic effect, and an anti-angiogenesis effect.

“Anti-migratory” refers to the ability of AMOs to stop cells from migrating away from the neoplastic tumor tissue and reducing the colonization of new tissues by such cells.

“Cancer” refers to the physiological condition in mammals typically characterized by unregulated cell growth/proliferation.

“Cancer cell death” refers to cell death, e.g., via apoptosis or necrosis, of a cancer cell.

“Cancer or tumor progression” refers to progression one or more stages of a cancer or a tumor, including tumorigenesis, growth and proliferation, inflammation, invasion, angiogenesis, migration, and metastasis.

“Cellular RNAs” are RNAs inherent to a cell and include protein coding RNAs and non-coding RNAs (ncRNA). Protein coding RNAs, e.g., mRNA, code for proteins and undergo translation to produce proteins. non-coding RNA (ncRNA) represent a variety of functional RNAs that do not undergo translation. Non-limiting examples of ncRNAs include tRNA, rRNA, snRNA, snoRNA, SRP RNA, asRNA, miRNA, siRNA, Y RNA, and telomerase RNA. lncRNAs (long non-coding RNAs) are non-protein coding transcripts longer than 200 nucleotides. tRNAs typically carry amino acids and deliver them to a ribosome. rRNAs typically couple with ribosomal proteins and participate in translation of mRNA to produce protein molecules. snRNAs are typically involved in splicing and other nuclear functions. snoRNAs are typically involved in nucleotide modification. SRP RNAs are typically involved in membrane integration. asRNAs are typically involved in transcription attenuation, mRNA degradation, mRNA stabilization, and translation blockage. Telomerase RNAs are typically involved in telomere synthesis.

“Extracellular vesicles” refer to membrane-derived microvesicles, which includes a range of vesicles, including exosomes, microparticles and shed microvesicles secreted by many cell types under both normal physiological and pathological conditions. The methods and compositions described herein can be applied to microvesicles of various sizes; for example, 30 to 200 nm, for example, 30 to 800 nm, for example, up to 2 um.

“Inhibiting cancer or tumor progression” means inhibiting the development, growth, proliferation, or spreading of a tumor, including, but not limited to: inhibition of growth of cells in a tumor; inhibition of tumor growth; reduction in the number of tumor cells; reduction in tumor size; inhibition of tumor cell infiltration into adjacent peripheral organs and/or tissues; inhibition of metastasis; increased length of survival of a patient following treatment; and/or decreased mortality of a patient at a given time point following treatment.

“Inhibiting cancer or tumor cell growth or proliferation” means decreasing a cancer or tumor cell's growth or proliferation by at least 10%, 20%, 30%, 40%, 50%, 60%, 70%, 80%, 90%, 95%, or 100%, and includes inducing cell death in a cell or cells within a tumor.

“Migration” is the process of cells migrating away from a neoplastic tumor tissue and colonizing new tissues, i.e., the metastatic process.

“miRNAs” are small endogenous noncoding RNA gene products about 22nt long that regulate gene expression in a sequence-specific manner by RNA interference (RNAi). miRNAs regulate the translation and degradation of mRNAs through base pairing to partially complementary sites, predominately in the untranslated region of mRNAs. miRNAs are expressed as long precursor RNAs. Drosha, an RNAse III endonuclease, processes many primary miRNAs in the nucleus, releasing ˜70nt precursor miRNAs. Drosha associates with DGCR8, a dsRNA-binding protein, to form a microprocessor complex. Precursor miRNAs can be transported to the cytoplasm by exportin-5 and cleaved by Dicer, an RNAse III endonuclease, releasing 17-24nt mature ds-miRNA. One strand of the miRNA duplex is incorporated into the effector complex RNA-induced silencing complex (RISC) that mediates target gene expression. Argonaute 2, a key component of RISC, may function as an endonuclease that cleaves target mRNAs.

“Normal cell death” refers to cell death, e.g., via apoptosis or necrosis, of a non-cancer cell.

“Nucleic acid” is used in its broadest sense and comprises ribonucleic acids (RNA) and deoxyribonucleic acids (DNA) from all possible sources, in all lengths and configurations, such as double-stranded, single-stranded, circular, linear or branched. All sub-units and sub-types are also comprised, such as oligomers, plasmids, viral and bacterial nucleic acids, as well as genomic and non-genomic DNA and RNA from animal and plant cells or other eukaryotes or prokaryotes, messenger RNA (mRNA) in processed and unprocessed form, transfer RNA (tRNA), heterogeneous nuclear RNA (hnRNA), ribosomal RNA (rRNA), mitochondrial RNA (mtRNA), nRNA (nuclear RNA), siRNA (short interfering RNA), small nuclear RNA (snRNA), small nucleolar RNA (snoRNA), small Cajal Body specific RNA (scaRNA), micro RNA (miRNA), doubled-stranded RNA (dsRNA), ribozyme, riboswitch, viral RNA, double-stranded DNA (dsDNA), single-stranded DNA (ssDNA), plasmid DNA, cosmid DNA, chromosomal DNA, viral DNA, mitochondrial DNA (mtDNA), nuclear DNA (nDNA), small nuclear DNA (snDNA), signal recognition particle RNA (SRP RNA), antisense RNA (asRNA), Y RNA, telomerase RNA, or the like.

“Patient”, “subject” and “individual” are used interchangeably herein, and refer to an animal, particularly a human, to whom treatment including prophylactic treatment is provided. This includes human and non-human animals.

“Peptide”, “polypeptide” and “protein” are used interchangeably to refer to amino acid sequences i.e., two or more amino acids linked by a peptide bond.

A “primer” refers to a natural or synthetic nucleic acid, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH.

“Purified” when used in reference to a microvesicle/extracellular vesicle refers to the fact that it is removed from the majority of other cellular components from which it was generated or in which it is typically present in nature.

“siRNA” is an agent which functions to inhibit expression of a target gene by RNA interference (RNAi). siRNA forms a double stranded RNA and has the ability to reduce or inhibit expression of a gene or target gene when the siRNA is present or expressed in the same cell as the target gene.

“Target nucleic acid” or “target polynucleotide” or “target RNA” refers to a nucleic acid molecule that has a function which is desired to be inhibited.

“Therapeutically effective amount” refers to an amount that is sufficient to effect a therapeutically significant reduction in one or more symptoms of a condition when administered to a typical subject who has the condition. A therapeutically significant reduction in a symptom is, e.g. about 10%, about 20%, about 30%, about 40%, about 50%, about 60%, about 70%, about 80%, about 90%, about 100%, or more as compared to a control or non-treated subject.

“Transfection” refers to the introduction of nucleic acid into a cell or extracellular vesicle (e.g., for the purpose of introducing an AMO. Examples of methods of transfection include, but are not limited to, electroporation, calcium phosphate, lipofection, and viral infection utilizing a viral vector. An AMO can be introduced into a cell or EV in a non-expressible form. An AMO can be introduced into a cell or EV in an expressible form (e.g., within an expression vector).

“Treat” or “treatment” refers to a therapeutic treatment wherein the object is to eliminate or lessen symptoms. Beneficial or desired clinical results include, but are not limited to, elimination of symptoms, alleviation of symptoms, diminishment of extent of a condition, stabilization (i.e., not worsening) of a condition's state, and delaying or slowing of progression of a condition.

“Tumor” refers to all neoplastic cell growth and proliferation, whether malignant or benign, and all pre-cancerous and cancerous cells and tissues.

### Target Polynucleotides

It is an object of the invention to inhibit the function of a target polynucleotide. The inventors have discovered that EVs contain one or more active target polynucleotide that can trigger primary cell death. hY RNAs were found to be significantly up-regulated in human cancer tissues, compared to normal tissues. Y RNAs are small RNAs (sRNAs) with poorly characterized functions, but are thought to be involved in RNA processing and DNA replication. The Y RNA family consists of four genes in humans (hY1, hY3, hY4, hY5) and two genes in mice (mY1 and mY3) that are transcribed by RNA polymerase III. Their primary transcripts range in length from about 83-112nt. The sizes of the human Y RNAs are 112nt (hY1), 101nt (hY3), 98nt (hY4), and 84nt (hY5). The secondary structure of Y RNAs is characterized by a large internal loop and a stem structure formed by base-pairing between the highly conserved 5′ and 3′-ends. Internal and external loops in Y RNAs may be accessible to nucleases that cleave single-stranded RNA and full-length Y RNA transcripts may be cleaved in the internal or external loops to generate 5′-Y RNA fragments. The RNA genes in this family exhibit are evolutionary conserved and have high sequence similarity in all vertebrates and invertebrates. Additionally, 966 hY RNA pseudogenes exist, of which hY5 has 8 in the human genome. The hY RNAs interact with both Ro60 and La proteins in ribonucleoprotein complexes found in normal and in systemic Lupus Erythematosus and Sjogren Syndrome samples. Y RNAs may have multiple functions based on the protein-partners present in the complexes. Cellular Y RNAs may have specific functional roles in forming part of the initiation of DNA replication complex, chaperoning misfolded RNAs, and maintaining 5S ribosomal RNAs. A variety of distinct proteins correlated with each of these functional roles may be associated with the Y RNAs.

Unexpectedly, the inventors have also discovered that fragments of an 83nt primary transcript of a human hY5 gene are generated within EVs and can include 29-31nt and 22-23nt processed products of primary hY5 RNA transcripts. Primary cells treated with cancer cell EVs exhibited rapid cell death in a dose dependent manner. Primary cells treated with deproteinized total RNA from EVs, or 31nt and 23nt synthetic versions of processed hY5 RNA also exhibited rapid cell death in a dose dependent manner A double stranded version of a processed hY5 product caused a substantially lower cell death phenotype compared to a single stranded version, unlike that as was seen with antiviral innate immune responses.

Processed hY5 product (e.g., 31nt and 23nt sRNAs) can be detected in EVs from both primary cells and cancer cells; however, exposure of EVs isolated from primary cells does not trigger cell death in the primary cells. As mentioned above, total EV RNAs treated with phenol (deproteinized) obtained from either primary or cancer cells, in addition to synthetic versions of processed hY5 products (e.g., 31nt or 23nt sRNAs), caused cell death when contacted to primary cells. Thus, In some embodiments, different analytes (e.g., proteins, nucleic acids, co-factors, etc.) present in primary and cancer cell EVs can be associated with of a processed hY5 cargo (e.g., 31nt or 23nt cargos) depending on their origin. For example, an analyte present in a sufficient amount in cancer cell EV to inhibit an inhibitor of the function of processed hY5 products may not be present in a sufficient amount in primary cell EVs to inhibit the functional inhibitor of processed hY5 products. For example, an analyte present in a sufficient amount in primary cell EVs to inhibit the function of processed hY5 products may not be present in a sufficient amount in cancer cell EVs to inhibit the function of processed hY5 products.

In some embodiments, a target polynucleotide is RNA. In some embodiments, a target polynucleotide is sRNA. In some embodiments, a target polynucleotide is transcribed by RNA polymerase III. In some embodiments, a target polynucleotide is a hY5 polynucleotide. In some embodiments, a target polynucleotide is hY5 RNA.

In one aspect, a target polynucleotide is a fragment of a primary transcript. In some embodiments, a target polynucleotide is a fragment of a primary hY5 RNA transcript, e.g., a full length transcript. In some embodiments, a target polynucleotide is specifically generated in an EV. In some embodiments, a target polynucleotide is generated in cancer cell EVs and primary cell EVs. In some embodiments, a target polynucleotide is not generated in a cell. In some embodiments, a target polynucleotide is not generated in a cancer cell. In some embodiments, a target polynucleotide is not generated in a primary cell. In some embodiments, a target polynucleotide is a fragment of a primary transcript wherein the fragment is not generated in a cell. In some embodiments, a target polynucleotide is a fragment of a primary transcript specifically generated in an EV. In some embodiments, a target polynucleotide is a fragment of a primary transcript generated in cancer cell EVs and primary cell EVs. In some embodiments, a target polynucleotide is a 5′ fragment. In some embodiments, a target polynucleotide is a hY5 RNA fragment. In some embodiments, a target polynucleotide is a 5′-hY5 RNA fragment.

In some embodiments, a target polynucleotide lacks a 5′-triphosphate or poly-uracil or poly-adenylyl group, e.g., those frequently found in RNA viral genomes. For example, a target polynucleotide can be a single stranded hY5 31nt and 23nt processed sRNA that lack a 5′-triphosphate or poly-uracil or -adenylyl strings. In some embodiments, single stranded hY5 31nt and 23nt processed sRNA are compartmentalized within EVs produced from a cancer cell. In some embodiments, a primary transcript of a target polynucleotide forms a stable hairpin structure and triggers substantially lower cell death.

In some embodiments, a target polynucleotide comprises a core sequence comprising 2 or more nucleic acids critical to an activity or function of the target polynucleotide. For example, deletion or rearrangement of these nucleotides can render a cancer cell EV containing such a processed sRNA much less effective in causing primary cell death For example, a target polynucleotide can comprise a core sequence comprising 3, 4, 5, 6, 7, 8, 9, 10, 11, 12, 13, 14, 15, 16, 17, 18, 19, 20 or more nucleic acids critical to an activity or function of the target polynucleotide In some embodiments, a target polynucleotide comprises a core sequence comprising 7, 8, or 9 nucleic acids critical to an activity or function of the target polynucleotide. In some embodiments, a target polynucleotide comprises a core sequence comprising 8 nucleic acids critical to an activity or function of the target polynucleotide. In some embodiments, a target polynucleotide can be a hY5 RNA fragment comprising a core nucleotide sequence critical to cancer cell EV induced primary cell death. In some embodiments, a core nucleic acid sequence present in both the 31nt and 23nt processed products critical in triggering the cell death phenotype is about 8 nucleotides in length. In the absence or introduction of variation in the eight nucleotide sequence, sRNA maintaining the remainder of the sequence found in hY5 and keeping a hairpin structure has residual negative selection for primary cells. Thus, a secondary structure of the 31nt sRNA may also be important. In some embodiments, a target polynucleotide comprises a secondary structure important to the activity or function of the target polynucleotide

In some embodiments, a double stranded version of a core nucleotide sequence found in a target polynucleotide is sufficient for hY1-dependent initiation of DNA replication. For example, a double stranded version of an eight nucleotide core sequence of hY5 sRNA (5′GUAGUGGG3′) is sufficient for hY1-dependent initiation of DNA replication.

In some embodiments, a target polynucleotide causes inappropriate and uncontrolled DNA replication signals in primary cells, and cause increased primary cell death. Such processed hY5-stimulated cell death signals can be less effective in inducing apoptosis in cancer cell lines given their characteristic loss of DNA replication controls inherent with transformed cells. In some embodiments, a target polynucleotide cause more cell death to primary cells than to cancer cells. In some embodiments, a target polynucleotide causes increased cell death only when its complementary strands are missing. In some embodiments, the cell death of primary cells is related to the amount of a target polynucleotide produced (e.g., a 5′-31nt hY5 fragment).

However, not all exposed primary cells may die. Different proportions of primary cells may survive depending on the primary cell type and dosage used. These results appear to indicate that not all co-cultured cells are equally sensitive. Tumor-fibroblast interactions may act in parallel to promote tumorigenicity. Further, not all associated primary fibroblast cells may be involved in this cooperative activity. Thus, provided herein is a method comprising contacting a primary cell population with a cancer cell EV or a hY5 fragments (e.g., 31nt product); and determining if surviving primary cells after treatment continue to fail to respond to the exposure of the 31nt or cancer cell EVs; or if they do provide support for tumor growth.

In another aspect, a target polynucleotide is a primary transcript. In some embodiments, a target polynucleotide is a primary transcript in a cell. In some embodiments, a target polynucleotide is a primary transcript in a cancer cell. In some embodiments, a target polynucleotide is a primary hY5 RNA transcript. In some embodiments, a target polynucleotide is a primary hY5 RNA transcript in a cancer cell. In some embodiments, a target polynucleotide that is a primary transcript can be inhibited, for example, using siRNA or asRNA technologies.

An exemplary target polynucleotide comprises the sequence 5′GUU GUG GG3′ (SEQ ID NO:1). An exemplary target polynucleotide comprises the sequence 5′AGU UGG UCC GAG UGU UGU GGG UUA UUG UUA A3′ (SEQ ID NO:2). An exemplary target polynucleotide comprises the sequence 5′-AGU UGG UCC GAG UGU UGU GGGUU-3′ (SEQ ID NO:3).

An exemplary target polynucleotide comprises the sequence 5′-AGU UGG UCC GAG UGU UGU GGG UU-3′ (SEQ ID NO:4).

An exemplary non-functional version of a target polynucleotide comprises the sequence

5′-AGU UGG UCC GAG UAC GUA CAG UUA UUG UUA A-3′ (SEQ ID NO:5).

An exemplary sequence of a primary transcript from which a target polynucleotide is derived, is 5′AGU UGG UCC GAG UGU UGU GGG UUA UUG UUA AGU UGA UUUA ACA UUG UCU CCC CCC ACA ACC GCG CUU GAC UAG CUU GCU GUU U-3′ (SEQ ID NO: 6). In some embodiments, the primary transcript from which a target polynucleotide is derived is not a target polynuceotide. In some embodiments, the primary transcript from which a target polynucleotide is derived is a target polynuceotide.

### Therapeutic Compositions

Provided herein are inhibitors of target polynucleotides that can be used in the provided compositions and methods. As used herein, an inhibitor of a target polynucleotide refers to an agent or compound that inhibits a target polynucleotide directly or indirectly. In some embodiments, an inhibitor of a target polynucleotide inhibits the function or an activity of the target polynucleotide. In some embodiments, an inhibitor of a target polynucleotide may not inhibit the expression of the target polynucleotide. For example, an inhibitor of a hY5 fragment can inhibit the function or activity of the hY5 fragment. For example, an inhibitor of a hY5 fragment can inhibit tumor progression mediated by the hY5 fragment. Inhibitors of target polynucleotides, e.g., inhibitors of hY5 fragments, include, but are not limited to a peptide, small molecule, nucleic acid, and antibody. Such inhibitors can be made using the nucleic acid sequences of target polynucleotides, e.g., processed hY5 products.

### Oligonucleotide Inhibitors

In one aspect, an inhibitor of a target polynucleotide is an oligonucleotide. In some embodiments, the oligonucleotide inhibitor comprises one or more chemical modifications to improve in vitro and in vivo stability or delivery.

In some embodiments, an inhibitor of a target polynucleotide interacts with the target polynucleotide directly. In some embodiments, an oligonucleotide inhibitor of a target polynucleotide is an oligonucleotide capable of inhibiting the function or masking a functional region of a target polynucleotide, e.g., an antisense masking oligonucleotide (AMO). In some embodiments, an inhibitor of a target polynucleotide interacts with a non-mRNA target polynucleotide directly. In some embodiments, inhibitors of target polynucleotides, e.g., AMOs, do not bind to target polynucleotide that is an mRNA.

An oligonucleotide inhibitor of a target polynucleotide can be designed to interact with a target polynucleotide based on sequence homology between the target polynucleotide and the oligonucleotide inhibitor. The oligonucleotide inhibitor can comprise a full length or truncated complimentary sequence to a target polynucleotide, e.g., a hY5 fragment. In some embodiments, an inhibitor of a target polynucleotide is an oligonucleotide capable of inhibiting the function or masking a functional region of a target polynucleotide, e.g., an antisense masking oligonucleotide (AMO). In some embodiments, an oligonucleotide inhibitor is from about 6 to 22 nucleotides in length, or is from about 10 to 18 nucleotides in length, or is about 11 to about 16 nucleotides in length. In some embodiments, an oligonucleotide inhibitor is about 14, 15, 16, or 17 nucleotides in length. In some embodiments, the oligonucleotide inhibitor consists of from 12 to 25 nucleobases, from 15 to 20 nucleobases or from 8 to 15 nucleobases.

AMOs can be designed based on the sequence of the target molecule. AMOs can be designed to interact with a target nucleic acid molecule through either canonical or non-canonical base pairing. The AMO may have exact sequence complementary to the target sequence or near complementarity. Thus, based on the sequence of a hY5 fragment, inhibitory oligonucleotides can be designed to bind to any form of a hY5 fragment. Inhibitory oligonucleotides typically bind to at least a portion of the target polynucleotide, in this case a hY5 fragment. The inhibitory nucleic acids are at least partially complementary to a hY5 fragment. In some embodiments, the oligonucleotide inhibitor is at least 90%, at least 95%, at least 98%, at least 99%, or 100% complementary to a portion of a target polynucleotide. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. A percent complementarity indicates the percentage of residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). 100% complementary means that all the residues of a nucleic acid sequence will hydrogen bond with the same number of residues in a second nucleic acid sequence. The sequence of an oligonucleotide inhibitor need not be 100% complementary to that of its target nucleic acid to hybridize. Thus, the hY5 fragment inhibitor sequence can have 100%, 95%, 90%, 85%, 80%, 75%, 70% complementarity, or any percent complementarity between 100% and 70%, to the sequence of a hY5 fragment. In some embodiments, AMOs can comprise at least 70%, at least 80%, at least 90%, at least 95%, or at least 99% sequence complementarity to a target region within the target nucleic acid sequence to which they are targeted. For example, an AMO in which 18 of 20 nucleobases of the oligomeric compound are complementary to a target region, and would therefore specifically hybridize, would represent 90 percent complementarity. In this example, the remaining noncomplementary nucleobases may be clustered together or interspersed with complementary nucleobases and need not be contiguous to each other or to complementary nucleobases. Percent complementarity of an AMO with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Optionally, a first portion of the hY5 fragment inhibitor sequence is identical (i.e., has 100% complementary) to sequence of a hY5 fragment, while a second portion of the hY5 fragment inhibitor sequence has less than 100% complementarity, e.g. 50%, to the sequence of a hY5 fragment.

AMOs are designed so that they bind (hybridize) to a target polynucleotide (e.g., a targeted portion of a hY5 fragment) and remain hybridized under physiological conditions. Typically, if they hybridize to a site other than the intended (targeted) polynucleotide sequence, they hybridize to a limited number of sequences that are not a target polynucleotide (to a few sites other than a target polynucleotide). Design of an AMO can take into consideration the occurrence of the nucleic acid sequence of the targeted portion of the target polynucleotide or a sufficiently similar nucleic acid sequence in other locations in the genome or transcriptome, such that the likelihood the AMO will bind other sites and cause “off-target” effects is limited. In some embodiments, AMOs that inhibit hY5 fragments can be designed and made using standard nucleic acid synthesis techniques. In some embodiments, an AMO is single-stranded. In some embodiments, an AMO comprises RNA. In some embodiments, an AMO comprises DNA. In some embodiments, an AMO comprises DNA and RNA.

An AMO need not hybridize to all nucleobases in a target sequence and the nucleobases to which it does hybridize may be contiguous or noncontiguous. AMOs may hybridize over one or more segments of a target polynucleotide, such that intervening or adjacent segments are not involved in the hybridization event (e.g., a loop structure or hairpin structure may be formed). In some embodiments, an AMO hybridizes to noncontiguous nucleobases in a target polynucleotide. For example, an AMO can hybridize to nucleobases in a target polynucleotide that are separated by one or more nucleobases to which the AMO does not hybridize.

Any of the AMOs or any component of an AMO (e.g., a nucleobase, sugar moiety, backbone) described herein may be modified in order to achieve desired properties or activities of the AMO or reduce undesired properties or activities of the AMO. For example, an AMO or one or more component of any AMO may be modified to enhance binding affinity to a target sequence on target polynucleotide; reduce binding to any non-target sequence; reduce degradation by cellular nucleases (i.e., RNAse H); improve uptake of the AMO into a cell and/or into the nucleus of a cell; alter the pharmacokinetics or pharmacodynamics of the AMO; and modulate the half-life of the AMO.

In some embodiments, an AMO has nucleotide analogues, including derivatives wherein the sugar is modified, as in 2′-O-methyl, 2′-deoxy-2′-fluoro, and 2′,3′-dideoxynucleoside derivatives, nucleic acid analogs based on other sugar backbones, such as threose, locked nucleic acid derivatives, bicyclo sugars, or hexose, glycerol and glycol sugars, nucleic acid analogs based on non-ionic backbones, such as “peptide nucleic acids,” these nucleic acids and their analogs in non-linear topologies, such as dendrimers, comb-structures, and nanostructures, and these nucleic acids and their analogs carrying tags (e.g., fluorescent, functionalized, or binding) bound to their ends, sugars, or nucleobases.

In some embodiments, the AMO comprises one or more backbone modification. In some embodiments, the AMO comprises one or more sugar moiety modification. In some embodiments, the AMO comprises one or more backbone modification and one or more sugar moiety modification.

In some embodiments, the backbone of the AMO is modified by various chemical modifications to improve in vitro and in vivo stability and to improve the in vivo delivery of AMOs. Modifications of AMOs include, but are not limited to, 2′-O-methyl modifications, 2′-O-methyl modified ribose sugars with terminal phosphorothioates and a cholesterol group at the 3′ end, 2′-O-methoxyethyl (2′-MOE) modifications, 2′-fluoro modifications, and 2′,4′ methylene modifications (LNAs). Further exemplary inhibitory nucleic acids include modified oligonucleotides (2′-O-methylated or 2′-O-methoxyethyl), locked nucleic acids (LNA), morpholino oligonucleotides, peptide nucleic acids (PNAs), PNA-peptide conjugates, and LNA/2′-O-methylated oligonucleotide mixmers. In some embodiments, an AMO comprises a 2′-O-methyl modified ribose sugars with terminal phosphorothioates and a cholesterol group at the 3′ end (“antagomir”). For exemplary modifications see, e.g., Valòczi et al., Nucleic Acids Res. 32(22):e175 (2004) Fabiani and Gait, RNA 14:336-46 (2008); Lanford et al., Science 327(5962:198-201 (2010); Elmen et al., Nature 452:896-9 (2008); Gebert et al., Nucleic Acids Res. 42(1):609-21 (2013); Kloosterman et al., PLoS Biol 5(8):e203 (2007); and Elmen et al., Nucleic Acids Res. 36:1153-1162 (2008).

In some examples, each monomer of the AMO is modified in the same way, for example each linkage of the backbone of the AMO comprises a phosphorothioate linkage or each ribose sugar moiety comprises a 2′O-methyl modification. In some examples, a combination of different modifications may be desired. For example, an AMO can comprise a combination of phosphorodiamidate linkages and sugar moieties comprising morpholine rings (morpholinos).

The AMOs described herein can comprise nucleobases that are complementary to nucleobases present in a target polynucleotide, e.g., a hY5 polynucleotide. The nucleobase of an AMO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target polynucleotide. Examples of modified nucleobases include, without limitation, hypoxanthine, xanthine, 7-methylguanine, 5,6-dihydrouracil, 5-methylcytosine, and 5-hydroxymethoylcytosine.

The AMOs may comprise naturally-occurring nucleotides, nucleotide analogs, modified nucleotides, or any combination. Naturally occurring nucleotides include deoxyribonucleotides and ribonucleotides. Modified nucleotides include nucleotides with modified or substituted sugar groups and/or having a modified backbone. In some embodiments, all of the nucleotides of the AMO are modified nucleotides. For exemplary chemical modifications of AMOs or components of AMOs that are compatible with the methods and compositions described herein see U.S. Pat. Nos. 8,258,109 B2 and 5,656,612; and U.S. Patent Publication No. 2012/0190728.

A representative, non-limiting list of modified nucleobases includes 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), and pyridoindole cytidine (H-pyrido(3′,′: 4,5)pyrrolo[2,3-d]pyrimidin-2-one). Modified nucleobases can also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808; those disclosed in The Concise Encyclopedia of Polymer Science And Engineering, pages 858-859, Kroschwitz, J. I., ed. John Wiley & Sons, 1990; those disclosed by Englisch et al., Angewandte Chemie, International Edition, 1991, 30, 613; and those disclosed by Sanghvi, Y. S., Chapter 15, Antisense Research and Applications, pages 289-302, Crooke, S. T. and Lebleu, B., ed., CRC Press, 1993. Certain of these nucleobases can be used for increasing the binding affinity of the AMOs described herein. These include 5-substituted pyrimidines, 6-azapyrimidines and N-2, N-6 and O-6 substituted purines, including 2-aminopropyladenine, 5-propynyluracil and 5-propynylcytosine. Modified nucleobases and their use are described, in U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692. Polycyclic heterocyclic compounds can be used in place of one or more of the naturally-occurring heterocyclic base moieties. These compounds can be used in to increase the binding properties of the AMO to a target polynucleotide. Modifications can be targeted to guanosines (G-clamps) or cytidine analogs. Representative cytosine analogs that make 3 hydrogen bonds with a guanosine in a second strand include 1,3-diazaphenoxazine-2-one, 1,3-diazaphenothiazine-2-one, and 6,7,8,9-tetrafluoro-1,3-diazaphenoxazine-2-one. These base modifications can hybridize with complementary guanine and the latter can hybridize with adenine and enhance helical thermal stability by extended stacking interactions. (see U.S. Pub. Nos. 2003/0207804 and 2003/0175906). In some embodiments, an AMO has one or more carboxamido-modified bases as described in PCT/US11/59588,

In some embodiments, an AMO comprises a locked nucleic acid (LNA) nucleotide analogue. Some embodiments of LNA nucleotide analogues are bicyclic nucleic acid analogs that contain one or more 2′-O, 4′-C methylene linkages, which effectively lock the furanose ring in a C3′-endo conformation. This methylene linkage “bridge” restricts the flexibility of the ribofuranose ring and locks the structure into a rigid bicyclic formation. ASOs comprising LNA nucleotide analogues can demonstrate a much greater affinity and specificity to their target polynucleotide than do natural DNA counterparts. LNAs can hybridize to complementary nucleic acids even under adverse conditions, such as under low salt concentrations. See, e.g., U.S. Pat. Nos. 6,130,038, 6,268,490, and 6,670,461.

In some embodiments, an AMO comprises a peptide nucleic acid (PNA) nucleotide analogue. In some embodiments of PNA nucleotide analogues, the negatively charged sugar-phosphate backbone of DNA can be replaced by a neutral polyamide backbone composed of N-(2-aminoethyl) glycine units. The chemical configuration of PNA typically enables the nucleotide bases to be positioned in approximately the same place as in natural DNA, allowing PNA to hybridize with complementary DNA or RNA sequence.

In some embodiments, an AMO comprises a glycol nucleic acid (GNA) nucleotide analogue (Zhang, L et al (2005), a simple glycol nucleic acid, (J. Am, Chem. Soc. 127:4174-4175), a threose nucleic acid (TNA) nucleotide analogue (Wu et al, Organic Letters, 2002, 4(8):1279-1282), a tricyclic nucleoside analog (Steffens et al, Helv Chim Acta (1997) 80:2426-2439; Steffens et al, J Am Chem Soc (1999) 121: 3249-3255; Renneberg et al, J Am Chem Soc (2002) 124: 5993-6002; and Renneberg et al, Nucl Acids Res (2002) 30: 2751-2757), or a phosphonomonoester nucleic acid which incorporates a phosphorus group in the backbone, for example, analogues with phosphonoacetate and thiophosphonoacetate internucleoside linkages (see, e.g., US Pat. Pub. No. 2005/0106598). In some embodiments, an AMO comprises a cyclobutyl ring replaces a naturally occurring furanosyl ring.

Any of the AMOs described herein may contain a sugar moiety that comprises ribose or deoxyribose, as present in naturally occurring nucleotides, or a modified sugar moiety or sugar analog, including a morpholine ring. In some embodiments, an AMO comprises at least one modified sugar moiety. In some embodiments, each sugar moiety is a modified sugar moiety. Non-limiting examples of modified sugar moieties include 2′ substitutions such as 2′-O-methyl (2′-O-Me), 2′-O-methoxyethyl (2′MOE), 2′-O-aminoethyl, 2′F; N3′-P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, an AMO has a phosphorodiamidate morpholino (PMO), a locked nucleic acid (LNA), a peptide nucleic acid (PNA), a 2′-O-methyl (2′-O-Me), a 2′-Fluoro (2′F), or a 2′-O-methoxyethyl (2′MOE) moiety. In some embodiments, an AMO has 2′-O-(2-methoxyethyl) (MOE) phosphorothioate-modified nucleotides.

In some embodiments, an AMO has a 2′ modification with respect to a 2′ hydroxyl. For example, the 2′ modification may be 2′ deoxy. Incorporation of 2′-modified nucleotides in AMOs may increase resistance to nucleases and thermal stability with target polynucleotides. Various modifications at the 2′ positions may be independently selected from those that provide increased nuclease sensitivity, without compromising molecular interactions with the target polynucleotide. Such modifications may be selected on the basis of their increased potency in vitro or in vivo. Exemplary methods for determining increased potency (e.g., IC₅₀) for target polynucleotide inhibition are described herein.

In some embodiments, the 2′ modification may be independently selected from O-alkyl (which may be substituted), halo, and deoxy (H). In some embodiments, substantially all, or all, nucleotide 2′ positions of the AMOs can be modified, e.g., as independently selected from O-alkyl (e.g., O-methyl), halo (e.g., fluoro), deoxy (H), and amino. For example, the 2′ modifications may each be independently selected from O-methyl and fluoro. In exemplary embodiments, purine nucleotides each have a 2′ OMe and pyrimidine nucleotides each have a 2′ F. In some embodiments, from one to about 20 2′ positions, or from about one to about ten 2′ positions, or from about one to about five, or from about one to about 2 or 3 2′ positions are left unmodified (e.g., as 2′ hydroxyls).

2′ modifications also include small hydrocarbon substituents. The hydrocarbon substituents include alkyl, alkenyl, alkynyl, and alkoxyalkyl, where the alkyl (including the alkyl portion of alkoxy), alkenyl and alkynyl may be substituted or unsubstituted. The alkyl, alkenyl, and alkynyl may be C₁ to C₁₀ alkyl, alkenyl or alkynyl, such as C₁, C₂, or C₃. The hydrocarbon substituents may include one or two or three non-carbon atoms, which may be independently selected from N, O, and/or S. The 2′ modifications may further include the alkyl, alkenyl, and alkynyl as O-alkyl, O-alkenyl, and O-alkynyl. Exemplary 2′ modifications include 2′-O-alkyl (C₁-C₃ alkyl, such as 2′OMe or 2′OEt), 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) substitutions.

In some embodiments, an AMO has at least one 2′-halo modification (e.g., in place of a 2′ hydroxyl), such as 2′-fluoro, 2′-chloro, 2′-bromo, and 2′-iodo. In some embodiments, the 2′ halo modification is fluoro. The AMO can contain from 1 to about 5 2′-halo modifications (e.g., fluoro), or from 1 to about 3 2′-halo modifications (e.g., fluoro). In some embodiments, the AMO contains all 2′-fluoro nucleotides, or 2′-fluoro on all pyrimidine nucleotides. In some embodiments, the 2′-fluoro groups are independently di-, tri-, or unmethylated.

In some embodiments, an AMO has one or more 2′-deoxy modifications (e.g., H for 2′ hydroxyl), and in some embodiments, contains from about 2-10 2′-deoxy modifications, or contains 2′ deoxy at all positions. In some embodiments, an AMO has 2′ positions modified as 2′OMe. In some embodiments, an AMO has purine nucleotides modified at the 2′ position as 2′OMe.

The AMOs described herein comprise a backbone structure that connects the components of an oligomer. In naturally occurring oligonucleotides, the backbone comprises a 3′-5′ phosphodiester linkage connecting sugar moieties of the oligomer. The backbone structure or oligomer linkages of the AMOs described herein include, but are not limited to, phosphorothioate, phosphorodithioate, phosphoroselenoate, phosphorodiselenoate, phosphoroanilothioate, phosphoraniladate, phosphoramidate, and the like. See e.g., LaPlanche et al. Nucleic Acids Res. 14:9081 (1986); Stec et al. J. Am. Chem. Soc. 106:6077 (1984), Stein et al. Nucleic Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). The term AMO embodies oligonucleotides and any other oligomeric molecule that comprises nucleobases capable of hybridizing to a complementary nucleobase on a target polynucleotide, such as a sRNA, but does not comprise a sugar moiety, such as a peptide nucleic acid (PNA). In some embodiments, an AMO a backbone structure of the AMO does not contain phosphorous but rather contains peptide bonds, for example in a PNA, or linking groups including carbamate, amides, and linear and cyclic hydrocarbon groups. In some embodiments, an AMO has a backbone modification comprising a phosphorothioate linkage or a phosphorodiamidate linkage. In some embodiments, the backbone modification is a phosphothioate linkage. In some embodiments, the backbone modification is a phosphoramidate linkage. The AMO can contain one or more phosphorothioate linkages. Phosphorothioate linkages have been used to render oligonucleotides more resistant to nuclease cleavage. For example, the AMO may be fully phosphorothioate-linked or may contain about half or ¾ phosphorothioate linkages. For example, the AMO may be partially phosphorothioate-linked, for example, phosphorothioate linkages may alternate with phosphodiester linkages. In some embodiments, however, the AMO is fully phosphorothioate-linked. In other embodiments, the AMO has from one to five or one to three phosphate linkages.

In some embodiments, an AMO has at least one terminal modification or “cap”. The cap may be a 5′ and/or a 3′-cap structure, which include chemical modifications at either terminus of the AMO (with respect to terminal ribonucleotides), and including modifications at the linkage between the last two nucleotides on the 5′ end and the last two nucleotides on the 3′ end. The cap structure as can increase resistance of the AMO to exonucleases without compromising molecular interactions with the target polynucleotide. Such modifications may be selected on the basis of their increased potency in vitro or in vivo. The cap can be present at the 5′-terminus (5′-cap) or at the 3′-terminus (3′-cap) or can be present on both ends. In some embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety. The phosphorothioate or phosphorodithioate linkage(s), when part of a cap structure, are generally positioned between the two terminal nucleotides on the 5′ end and the two terminal nucleotides on the 3′ end.

In some embodiments, an AMO has at least one terminal phosphorothioate monophosphate. The phosphorothioate monophosphate may support a higher potency by inhibiting the action of exonucleases. The phosphorothioate monophosphate may be at the 5′ and/or 3′ end of the AMO.

In some embodiments, an AMO has phosphorothioate linkages between the last two nucleotides on the 5′ and the 3′ end (e.g., as part of a cap structure), or as alternating with phosphodiester bonds. In these or other embodiments, the AMO can contain at least one terminal abasic residue at either or both the 5′ and 3′ ends. An abasic moiety does not contain a commonly recognized purine or pyrimidine nucleotide base, such as adenosine, guanine, cytosine, uracil or thymine. Thus, such abasic moieties lack a nucleotide base or have other non-nucleotide base chemical groups at the 1′ position. For example, the abasic nucleotide may be a reverse abasic nucleotide, e.g., where a reverse abasic phosphoramidite is coupled via a 5′ amidite (instead of 3′ amidite) resulting in a 5′-5′ phosphate bond.

An exemplary target polynucleotide comprises the sequence 5′GUU GUG GG3′ (SEQ ID NO:1). An exemplary target polynucleotide comprises the sequence 5′AGU UGG UCC GAG UGU UGU GGG UUA UUG UUA A3′ (SEQ ID NO:2). An exemplary target polynucleotide comprises the sequence 5′-AGU UGG UCC GAG UGU UGU GGGUU-3′ (SEQ ID NO:3).

An exemplary target polynucleotide comprises the sequence 5′-AGU UGG UCC GAG UGU UGU GGG UU-3′ (SEQ ID NO:4).

An exemplary non-functional version of a target polynucleotide comprises the sequence 5′-AGU UGG UCC GAG UAC GUA CAG UUA UUG UUA A-3′ (SEQ ID NO:5).

An exemplary sequence of a primary transcript from which a target polynucleotide is derived, is 5′AGU UGG UCC GAG UGU UGU GGG UUA UUG UUA AGU UGA UUUA ACA UUG UCU CCC CCC ACA ACC GCG CUU GAC UAG CUU GCU GUU U-3′ (SEQ ID NO:6)

In some embodiments, an oligonucleotide inhibitor comprises the nucleotide sequence of 5′-CCC ACA AC-3′ (SEQ ID NO:7). In some embodiments, an oligonucleotide inhibitor comprises the nucleotide sequence of 5′=CCC CAC AAC CGC GCU UGA CUA GCU UGC UGU UU=3′ (SEQ ID NO:8). In some embodiments, an oligonucleotide inhibitor comprises the nucleotide sequence of 5′-CCC CAC AAC CGC GCT TGA CTA GCT TGC TGT TT-3′ (SEQ ID NO:9). In some embodiments, an oligonucleotide inhibitor comprises the nucleotide sequence of 5′-CCC ACA ACC GCG CUU GAC UAG CU-3′ (SEQ ID NO:10). In some embodiments, an oligonucleotide inhibitor comprises the nucleotide sequence of 5′-CCC ACA ACC GCG CTT GAC TAG CT-3′ (SEQ ID NO:11). In some embodiments, an oligonucleotide inhibitor comprises the nucleotide sequence of 5′-CCC ACA ACC GCG CUU GGA CUA GCU-3′ (SEQ ID NO:12). In some embodiments, an oligonucleotide inhibitor comprises the nucleotide sequence of 5′-CCC ACA ACC GCG CTT GGA CTA GCT-3′ (SEQ ID NO:13). In some embodiments, an oligonucleotide inhibitor comprises the nucleotide sequence of’ 5′-CCC ACA ACA CUU GAC UAG CU-3′ (SEQ ID NO:14). In some embodiments, an oligonucleotide inhibitor comprises the nucleotide sequence of’ 5′-CCC ACA ACA CTT GAC TAG CT-3′ (SEQ ID NO:15). In some embodiments, an oligonucleotide inhibitor comprises the nucleotide sequence of’ 5′-CCC ACA ACA CUU GGA CUA GCU-3′ (SEQ ID NO:16). In some embodiments, an oligonucleotide inhibitor comprises the nucleotide sequence of’ 5′-CCC ACA ACA CTT GGA CTA GCT-3′ (SEQ ID NO:17).

In some embodiments, an oligonucleotide inhibitor comprises the nucleotide sequence of 5′-UUA ACA UUG UCU CCC CCC ACA AC-3′ (SEQ ID NO:18). In some embodiments, an oligonucleotide inhibitor comprises the nucleotide sequence of 5′-TTA ACA TTG TCT CCC CCC ACA AC-3′ (SEQ ID NO:19). In some embodiments, an oligonucleotide inhibitor comprises the nucleotide sequence of 5′-UUA ACA AUA ACC CAC AAC-3′ (SEQ ID NO:20). In some embodiments, an oligonucleotide inhibitor comprises the nucleotide sequence of 5′-TTA ACA ATA ACC CAC AAC-3′ (SEQ ID NO:21).

Exemplary hY5 fragment inhibitors also include 5′-CcC ACa aC-3′ (SEQ ID NO: 7), with LNA in capitals, DNA in lower case, complete phosphorothioate backbone, and capital C denotes LNA methylcytosine. Other exemplary hY5 fragment inhibitors can comprise the sequence 5′-CcC cAc aaC CGC GCT TGA CTA GCT TGC TGT TT-3′ (SEQ ID NO:22). Other exemplary hY5 fragment inhibitors can comprise the sequence 5′CCC ACA ACC GCG CTT GAC TAG CT-3′ (SEQ ID NO:23). Other exemplary hY5 fragment inhibitors can comprise the sequence 5′CCC ACA ACC GCG CTT GGA CTA GCT-3′ (SEQ ID NO:24). Other exemplary hY5 fragment inhibitors can comprise the sequence 5′-CCC ACA ACA CTT GAC TAG CT-3′ (SEQ ID NO:25). Other exemplary hY5 fragment inhibitors can comprise the sequence 5′-CCC ACA ACA CTT GGA C TA GCT-3′ (SEQ ID NO:26). Other exemplary hY5 fragment inhibitors can comprise the sequence 5′-TTA ACA TTG TCT CCC CCC ACA AC-3′ (SEQ ID NO:27). Other exemplary hY5 fragment inhibitors can comprise the sequence 5′-TTA ACA ATA ACC CAC AAC-3′ (SEQ ID NO:28).

Exemplary hY5 fragment inhibitors also include 5′-C*C*C ACA*A*C-3′ (SEQ ID NO:29) being fully 2′-O-Me RNA and * indicates phosphorothioate linkage, Other exemplary hY5 fragment inhibitors can comprise the sequence 5′-C*C*C CAC AAC CGC GCU UGA CUA GCU UGC UG*U*U*U-3′ (SEQ ID NO:30). Other exemplary hY5 fragment inhibitors can comprise the sequence 5′-C*C*C ACA ACC GCG CUU GAC*U*A*G*C*U-3′ (SEQ ID NO:31). Other exemplary hY5 fragment inhibitors can comprise the sequence 5′-C*C*C*ACA ACC GCG CUU GGA CUA*G*C*U-3′ (SEQ ID NO:32). Other exemplary hY5 fragment inhibitors can comprise the sequence 5′-C*C*C ACA ACA CUU GAC*U*A*G*C*U-3′ (SEQ ID NO:33). Other exemplary hY5 fragment inhibitors can comprise the sequence 5′-C*C*C*ACA ACA CUU GGA CUA*G*C*U-3′ (SEQ ID NO:34). Other exemplary hY5 fragment inhibitors can comprise the sequence 5′-U*U*A*A*CA UUG UCU CCC CCC AC*A*A*C-3′ (SEQ ID NO:35). Other exemplary hY5 fragment inhibitors can comprise the sequence 5′-UUA ACA AUA ACC C*A*C*A*A*C-3′ (SEQ ID NO:36).

In some embodiments, inhibitors of target polynucleotides can be used as antisense constructs to control gene expression in cells, tissues or organs. In some embodiments, inhibitors of target polynucleotides bind to mRNA.

The methodology associated with antisense techniques is well known to the skilled artisan, and is described and reviewed in Antisense Drug Technology: Principles, Strategies, and Applications, Crooke, Marcel Dekker Inc., New York (2001). In general, antisense nucleic acids are designed to be complementary to a region of mRNA expressed by a gene, so that the antisense molecule hybridizes to the mRNA, thus blocking translation of the mRNA into protein. Several classes of antisense oligonucleotide are known to those skilled in the art, including cleavers and blockers. The former bind to target RNA sites, activate intracellular nucleases (e.g., RNAse H or RNAse L) that cleave the target RNA.

In some embodiments, an inhibitor of a target polynucleotide is an oligonucleotide that inhibits translation of a primary transcript of a target polynucleotide. For example, an inhibitor of a target polynucleotide can be an asRNA that inhibits translation of a primary hY5 transcript. In some embodiments, an inhibitor is an indirect inhibitor that inhibits translation of a protein that activates a target polynucleotide. For example, an inhibitor can be an asRNA that inhibits translation of an mRNA encoding a protein required for an activity or function of a hY5 fragment. In some embodiments, an inhibitor is an indirect inhibitor that inhibits translation of a protein that has a processing activity towards a primary transcript of a target polynucleotide. For example, an inhibitor can be an asRNA that inhibits translation of a complementary mRNA encoding a nuclease that cleaves a primary hY5 transcript to a 5′-hY5 fragment.

In some embodiments, an inhibitor of a target polynucleotide is an oligonucleotide that reduces expression or abundance of an mRNA encoding a protein that activates a target polynucleotide. For example, an inhibitor of a target polynucleotide can be a siRNA or miRNA that reduces the expression or abundance of an mRNA encoding a protein that activates a target polynucleotide. In some embodiments, an inhibitor is an indirect inhibitor that reduces expression or abundance of an mRNA encoding a protein that has a processing activity towards a primary transcript of a target polynucleotide. For example, an inhibitor can be a siRNA or miRNA that reduces expression or abundance of an mRNA encoding a nuclease that cleaves a primary hY5 transcript to a 5′-hY5 fragment.

In some embodiments, an inhibitor is an indirect inhibitor that interacts with a molecule that binds to a target polynucleotide, e.g., a hY5 fragment, to inhibit the activity or function of the target polynucleotide. In some embodiments, an inhibitor is an indirect inhibitor that interacts with molecule that binds to a target polynucleotide, e.g., a hY5 fragment, to reduce or eliminate the presence of the target polynucleotide. In some embodiments, an inhibitor of a target polynucleotide is a functional oligonucleotide, e.g., a ribozyme. Inhibitors of hY5 fragments can also include inhibitory polypeptides and antibodies.

The activity or potency of the oligonucleotide inhibitors may be determined in vitro and/or in vivo. For example, the oligonucleotide may significantly inhibit (e.g., about 50% inhibition) the activity or function of a target polynucleotide, e.g., a hY5 fragment, at a concentration of about 1 mM, 100 μM, 10 μM, 1 μM, 100 nm, 50 nM or less, or in other embodiments, 40 nM, 20 nM, or 10 nM or less. Alternatively, or in addition, the activity or function of the oligonucleotide may be determined in a suitable mouse or rat model, or non-human primate model, where inhibition (e.g., by at least 50%) of a μM is observed at a dose of 50 mg/kg or less, such as 25 mg/kg or less, 10 mg/kg or less, or 5 mg/kg or less. For example, the oligonucleotide may be dosed subcutaneously or intravenously and may be formulated in an aqueous preparation (e.g., saline).

Methods of Identifying AMOs that Inhibit Target Polynucleotides

Also within the scope of the present invention are methods for identifying AMOs that inhibit a target polynucleotide. AMOs that specifically hybridize to target polynucleotides may be screened to identify AMOs that inhibit target polynucleotide activity or function. Any method known in the art may be used to identify an AMO that when hybridized to the target polynucleotide results in the desired effect (e.g., inhibition of cell death or tumor progression caused by cancer cell EVs). An example of a method that may be used is provided below.

As a round of screening, an AMO “walk” may be performed using AMOs that have been designed to hybridize to a target region of a hY5 fragment. The AMOs used in the AMO walk are tiled every 1 nucleotide from an end of an hY5 fragment to the other end of the hY5 fragment. For example, a first AMO of 15 nucleotides in length may be designed to specifically hybridize to nucleotides+1 to +15 relative to the 3′ end of the hY5 fragment. A second AMO is designed to specifically hybridize to nucleotides+2 to +16 relative to the 3′ end of the hY5 fragment. For example, a first AMO of 15 nucleotides in length may be designed to specifically hybridize to nucleotides+1 to +15 relative to the 5′ end of the hY5 fragment. A second AMO is designed to specifically hybridize to nucleotides+2 to +16 relative to the 5′ end of the hY5 fragment.

In some embodiments, one or more AMOs, or a control AMO (an AMO with a scrambled sequence, sequence that is not expected to hybridize to the target region) are delivered, for example by transfection, into a cancer cell-derived EV that has the target polynucleotide, e.g., hY5 fragment.

AMOs that hybridize to a region target polynucleotide and inhibit an activity or function of the target polynucleotide (e.g., inhibition of cell death or tumor progression caused by cancer cell EVs) can be tested in vitro using cell cultures or tested in vivo using animal models. Suitable routes for administration of AMOs may vary depending on the disease and/or the cell types to which delivery of the AMOs is desired. AMOs may be administered, for example, by intravitreal injection, intrathecal injection, intraperitoneal injection, subcutaneous injection, or intravenous injection. Following administration, the cells, tissues, and/or organs of the model animals may be assessed to determine the effect of the AMO treatment. The animal models may also be any phenotypic or behavioral indication of the disease or disease severity.

A variety of different agents may be screened by the above methods. Candidate agents encompass numerous chemical classes including, but not limited to, peptides, polynucleotides (e.g., AMOs), and organic molecules (e g, small organic compounds having a molecular weight of more than 50 and less than about 2,500 Daltons). Candidate agents can comprise functional groups for structural interaction with target polynucleotides, such as hydrogen bonding, and can include at least one or at least two of an amine, carbonyl, hydroxyl or carboxyl group. The candidate agents can comprise cyclical carbon or heterocyclic structures and/or aromatic or polyaromatic structures substituted with one or more functional groups. Candidate agents can be biomolecules including peptides, saccharides, fatty acids, steroids, purines, pyrimidines, derivatives, structural analogs or combinations thereof. Candidate agents can be obtained from a wide variety of sources including libraries of synthetic or natural compounds. For example, numerous means are available for random and directed synthesis of a wide variety of organic compounds and biomolecules, including expression of randomized polynucleotides and polypeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs.

Furthermore, arrays may also be used in a method for screening agents. An array can be a high-density array. A high-density array can comprise tens, hundreds, thousands, tens-of-thousands or hundreds-of-thousands of candidate agents. The density of microspots of an array may be at least about 1/cm² or at least about 10/cm², up to about 500/cm² or up to about 1,000/cm². In some embodiments, the density of all the microspots on the surface of the substrate may be up to about 400/cm², up to about 300/cm², up to about 200/cm², up to about 100/cm², up to about 90/cm², up to about 80/cm², up to about 70/cm², up to about 60/cm², or up to about 50/cm². For example, an array can comprise at least 50, 60, 70, 80, 90, 100, 150, 200, 250, 300, 350, 400, 450, 500, 600, 700, 800, 900, or 1,000 distinct candidate agents per a surface area of less than about 1 cm². For example, an array can comprise 50, 60, 70, 80, 90, 100, 150, 200, 250, 300, 350 or 400 discrete regions in an area of about 16 mm², or 2,500 discrete regions/cm². In some embodiments, candidate agents on an array are screened directly for their ability to bind or otherwise interact with a target polynucleotide. A plurality of potential agents may be screened in parallel for their ability to bind or otherwise interact with a target polynucleotide. The screening process may involve assaying for the interaction, such as binding, of at least one agent with a hY5 fragment, for example, a hY5 RNA fragment from an EV produced by a cancer cell.

### Cells

In some embodiments of the presently disclosed subject matter, a cell that produces the extracellular vesicles comprising a target polynucleotide disclosed herein is provided. In some embodiments, the cell is a cultured cell, that is, a cell propagated ex vivo in culture media. The culture cell can be immortalized to facilitate continuous propagation. In some embodiments, the cell is a cancer cell, such as for example a cancer cell originally isolated from a tumor and then propagated in culture. In some embodiments, the cancer cell can be an ovarian cancer cell, a cervical cancer cell, a breast cancer cell, an endometrial cancer cell, a colon cancer cell, a prostate cancer cell, a lung cancer cell, a melanoma cell, or a pancreatic cancer cell.

### Diagnostic Methods

In some embodiments, extracellular vesicle preparations can be used as a diagnostic tool. For example, EVs can be isolated from a particular tissue, evaluated for their nucleic acid or protein content, which can then be correlated to disease state or risk of developing a disease.

Further, the presently disclosed subject matter provides for the isolation of cancer cell-derived EVs from a biological fluids from a test subject. As such, the presently disclosed subject matter provides methods for diagnosis and prognosis of cancer based on the collection and measurement of cancer-derived EV RNA levels or activity levels, e.g., hY5 RNA fragment levels from biological samples, and in some instances without necessitating direct sampling of cancer cells.

In some embodiments of the presently-disclosed subject matter, a method for assessing the presence or activity of one or more RNAs of a disease (e.g., a RNA signature or RNA expression profile) is provided. In some embodiments of the presently disclosed subject matter, a method is provided for assessing the presence or activity of one or more RNAs in EVs. In some embodiments the method involves isolating cancer cell-derived EVs from a sample, isolating sRNA from the cancer cell-derived EVs, and/or determining a presence or activity of one or more sRNAs in cancer cell-derived EVs. A circulating tumor-derived vesicle is a vesicle shed into circulation or bodily fluids from tumor cells. EVs can be directly assayed from a biological sample. The level or amount of vesicles in the sample, the bio-signature of one or more vesicles in the sample, or the presence or activity of one or more sRNAs can be determined without prior isolation, purification, or concentration of the biological sample, vesicles, or sRNAs. Alternatively, the EVs in the sample may be isolated, purified, or concentrated from a sample prior to analysis. In some embodiments, determining the presence of one or more sRNAs includes determining a fragment profile of the one or more target RNAs. The one or more target RNAs in the sample or the fragment profile of the one or more target RNAs in the sample can be compared to a reference. In some embodiments the sample can be a biological sample obtained from a subject. In some embodiments, the sample can be obtained from a cell culture.

In some embodiments of the presently disclosed subject matter, a method for characterizing a disease in a subject is provided. Characterizing can include providing a diagnosis, prognosis, and/or theranosis of the disease. In some embodiments, the method can include isolating cancer cell-derived EVs from a biological sample of the subject, determining an amount of one or more RNAs in the isolated cancer cell-derived EVs, and comparing the amount of the one or more RNAs to a reference, wherein the disease is characterized based on a measurable difference in the amount of the one or more RNAs or tumor grogression caused by the one or more RNAs from the cancer cell-derived EVs as compared to a control. For example, in some embodiments the subject can be diagnosed as having the disease or risk thereof if there is a measurable difference in the amount of the one or more target RNA fragments or tumor grogression caused by the one or more RNA fragments from the cancer cell-derived EVs in the sample as compared to a reference. For example, in some embodiments the subject can be diagnosed as having the disease or risk thereof if there is a measurable difference in an activity, e.g., a pro-apoptotic or pro-metastatic activity, of the one or more target RNA fragments or tumor grogression caused by the one or more RNA fragments from the cancer cell-derived EVs in the sample as compared to a reference.

In some embodiments, a method for evaluating treatment efficacy and/or progression of a disease in a subject is provided. In some embodiments, the method can involve isolating cancer cell-derived EVs from a biological sample of the subject, determining an amount of one or more target RNA fragments or tumor grogression caused by the one or more RNA fragments in the isolated cancer cell-derived EVs, and determining any measurable change in the amounts or activities of the one or more target RNA fragments to thereby evaluate treatment efficacy and/or progression of the cancer in the subject. In some embodiments, the biological sample can include a first biological sample collected prior to initiation of treatment for the disease and/or onset of the disease and a second biological sample collected after initiation of the treatment or onset. In some embodiments, the method can also include selecting a treatment or modifying a treatment for the disease based on the amount of the one or more target RNA fragments or tumor grogression caused by the one or more RNA fragments determined.

In some embodiments, a method for characterizing a cancer in a subject is provided and includes isolating EVs from a biological sample of the subject; determining a presence or an amount of one or more target RNA fragments from the isolated EVs; and comparing the presence, activity, tumor grogression caused by the one or more RNA fragments, or the amount of the one or more target RNA fragments to a reference, wherein the cancer is characterized based on a measurable difference in the presence, activity, tumor grogression caused by the one or more RNA fragments, or the amount of the one or more target RNA fragments from the isolated EVs as compared to the reference. In some embodiments, the characterizing comprises providing a diagnosis, prognosis and/or theranosis of the cancer. A biological sample can be used for the detection of the presence, activity, tumor grogression caused by the one or more RNA fragments, and/or fragment profile level of a sRNA, e.g., a hY5 RNA fragment, of interest associated with cancer-derived EVs. Any cell, group of cells, cell fragment, or cell product can be used with the methods of the presently claimed subject matter, although biological fluids and organs that would be predicted to contain cancer-derived EVs exhibiting differential activity, tumor grogression caused by the one or more RNA fragments, or levels of hY5 RNA fragments as compared to normal controls, e.g., EVs derived from non-cancerous cells or from a biological sample from a subject without cancer, are best suited. In some embodiments, the biological sample is blood or a component thereof. In some embodiments, the biological sample comprises milk, blood, serum, plasma, ascites, cyst fluid, pleural fluid, peritoneal fluid, cerebral spinal fluid, tears, urine, saliva, sputum, or combinations thereof.

### Methods of Isolating Vesicles

Compositions and methods of the invention are directed to assaying one or more vesicles. Vesicles include without limitation the following types or species: extracellular vesicle (EV), microvesicle, exosome, nanovesicle, dexosome, bleb, blebby, prostasome, microparticle, intralumenal vesicle, membrane fragment, intralumenal endosomal vesicle, endosomal-like vesicle, exocytosis vehicle, endosome vesicle, endosomal vesicle, apoptotic body, multivesicular body, secretory vesicle, phopholipid vesicle, liposomal vesicle, argosome, texasome, secresome, tolerosome, melanosome, oncosome, or exocytosed vehicle. Unless otherwise specified, methods that make use of a species of vesicle can be applied to other types of vesicles. Vesicles comprise spherical structures with a lipid bilayer similar to cell membranes which surrounds an inner compartment which can contain soluble components. In some embodiments, the methods of the invention make use of exosomes, which are small secreted vesicles of about 50-100 nm in diameter.

In some embodiments, the cancer cell-derived vesicles are isolated using size exclusion chromatography, PEG-precipitation of the vesicles, filtration, or immunosorbent capture. In some embodiments, isolating the vesicles comprises using an agarose-based gel. Size exclusion chromatography, PEG-precipitation, filtration, and immunosorbent capture techniques are known in the art

In some embodiments, a void volume fraction is isolated and comprises the vesicles of interest. Further, in some embodiments, the cancer cell-derived vesicles can be further isolated after chromatographic separation by centrifugation techniques (of one or more chromatography fractions), as is generally known in the art. In some embodiments, for example, density gradient centrifugation can be used to further isolate the vesicles. Still further, in some embodiments, it can be desirable to further separate the cancer-derived isolated vesicles from vesicles of other origin.

In some embodiments, cancer cell-derived vesicles are isolated using affinity selection. For example, cancer cell-derived vesicles can be isolated based on their affinity for particular binding agents. For example, a binding agent can be an antibody or an aptamer. Thus, binding agents can be used in affinity selection to select particular ligands, molecules, substances, or the like based on the extent to which they bind with a particular binding agent. In some embodiments, affinity selection comprises separating the cancer-cell-derived vesicles from non-cancer-derived EVs by immunosorbent capture using an anti-cancer antigen antibody as the binding agent.

In some embodiments, EVs are isolated from cellular preparations by methods comprising one or more of filtration, centrifugation, antigen-based capture and the like. In some embodiments, a population of cells grown in culture are collected and pooled. In some embodiments, monolayers of cells are pooled. In some embodiments, cells grown in suspension are used. In some embodiments, the pooled population is subject to one or more rounds of centrifugation e.g., ultracentrifugation and/or density centrifugation to separate the EV fraction from cells and cellular debris. In some embodiments, centrifugation need not be performed to harvest EVs. In some embodiments, size exclusion filtration is used in conjunction with, or in place of centrifugation, in order to collect a particular size (e.g., diameter) of EV. In some embodiments, filtration need not be used. In some embodiments, EVs are captured by affinity chromatography using agents that bind to unique markers on or in the EVs (e.g., transmembrane proteins)). In such instances, the unique markers can be used to selectively enrich a particular EV population, such as those derived from cancer cells.

In one aspect, a method of isolating EVs comprises centrifuging a cell medium at low speed. For example, 200 mL, of cellular medium can be centrifuged at 300 g for 10 min. The method can further comrpise removing a cell pellet after the centrifugation at low speed. The method can further comrpise centrifuging the supernatant of the low speed centrifugation step at a medium speed. For example, the supernatant can be centrifuged at 2,000 g for 10 min. The method can further comrpise removing a pellet of cell debris and apoptotic bodies that result from the second centrifugation step. The method can further comrpise centrifuging the supernatant of the second centrifugation step at high speed. For example, the supernatant can be centrifuged at 10,000 g for 30 min. The method can further comrpise removing a pellet containing resulting from the high speed centrifugation step. The method can further comrpise filtering the supernatant of the high speed centrifugation step with a membrane. For example, the supernatant of the high speed centrifugation step can be filtered with a Centricon Plus 70-100 KD (10 nm pore size approx.) centrifugal filter at 3500 g for 15 min The method can further comrpise collecting a retentate of the filtering step that is enriched in EVs, such as exosomes. The retentate can be resuspended in a volume of buffer. For example, the volume of the retentate can be resuspended in 500 μL of PBS. In some embodiments, the filtrate of the filtering step can be discarded. The volume of the filtration residue was made to 500 μL using PBS.

### Therapeutic Methods

Provided are methods of treating a disease or disorder in a subject, the method comprising administration to the subject a composition comprising an AMO described herein.

In some embodiments, the present invention provides compositions and methods for reducing the amount of a target nucleic acid or tumor grogression caused by the target nucleic acid in an EV derived from a cancer cell. An AMO can have anti-tumor activity. In some embodiments, a pharmaceutical composition is administered to an animal having at least one cancer cell. In some embodiments, a pharmaceutical composition is administered to an animal having at least one symptom associated with cancer. In some embodiments, such administration results in amelioration of at least one symptom. In some embodiments, the administration of an AMO delays the onset of cancer. In some embodiments, the administration of an AMO slows the proliferation of cancer cells. In some embodiments, the administration of an AMO slows the proliferation of tumor cells. In some embodiments, the administration of an AMO prevents the growth of cancer. In some embodiments, the administration of an AMO prevents the formation of tumors. In some embodiments, the administration of an AMO causes tumor mass to decrease. In some embodiments, the administration of an AMO rescues cellular phenotype.

In some embodiments, the methods for treating cancer provided herein inhibit, reduce, diminish, arrest, or stabilize a tumor associated with the cancer. In other embodiments, the methods for treating cancer provided herein inhibit, reduce, diminish, arrest, or stabilize the blood flow, metabolism, or edema in a tumor associated with the cancer or one or more symptoms thereof. In specific embodiments, the methods for treating cancer provided herein cause the regression of a tumor, tumor blood flow, tumor metabolism, or peritumor edema, and/or one or more symptoms associated with the cancer. In other embodiments, the methods for treating cancer provided herein maintain the size of the tumor so that it does not increase, or so that it increases by less than the increase of a tumor after administration of a standard therapy as measured by conventional methods available to one of skill in the art, such as digital rectal exam, ultrasound (e.g., transrectal ultrasound), CT Scan, MRI, dynamic contrast-enhanced MRI, or PET Scan. In specific embodiments, the methods for treating cancer provided herein decrease tumor size. In some embodiments, the methods for treating cancer provided herein reduce the formation of a tumor. In some embodiments, the methods for treating cancer provided herein eradicate, remove, or control primary, regional and/or metastatic tumors associated with the cancer. In some embodiments, the methods for treating cancer provided herein decrease the number or size of metastases associated with the cancer.

In some embodiments, the methods for treating cancer provided herein reduce the tumor size (e.g., volume or diameter) in a subject by at least about 5%, 10%, 15%, 20%, 25%, 30%, 35%, 40%, 45%, 50%, 55%, 60%, 65%, 80%, 85%, 90%, 95%, 99%, or 100%, relative to tumor size (e.g., volume or diameter) prior to administration of an AMO as assessed by methods well known in the art, e.g., CT Scan, MRI, DCE-MRI, or PET Scan. In particular embodiments, the methods for treating cancer provided herein reduce the tumor volume or tumor size (e.g., diameter) in a subject by an amount in the range of about 5% to 20%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 30% to 100%, or any range in between, relative to tumor size (e.g., diameter) in a subject prior to administration of an AMO as assessed by methods well known in the art, e.g., CT Scan, MRI, DCE-MRI, or PET Scan.

In some embodiments, the methods for treating cancer provided herein reduce the tumor perfusion in a subject by at least about 5%, 10%, 15%, 20%, 25%, 30%, 35%, 40%, 45%, 50%, 55%, 60%, 65%, 80%, 85%, 90%, 95%, 99%, or 100%, relative to tumor perfusion prior to administration of an AMO as assessed by methods well known in the art, e.g., MRI, DCE-MRI, or PET Scan. In particular embodiments, the methods for treating cancer provided herein reduce the tumor perfusion in a subject by an amount in the range of about 5% to 20%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 30% to 100%, or any range in between, relative to tumor perfusion prior to administration of an AMO, as assessed by methods well known in the art, e.g., MRI, DCE-MRI, or PET Scan.

In particular aspects, the methods for treating cancer provided herein inhibit or decrease tumor metabolism in a subject as assessed by methods well known in the art, e.g., PET scanning. In specific embodiments, the methods for treating cancer provided herein inhibit or decrease tumor metabolism in a subject by at least about 5%, 10%, 15%, 20%, 25%, 30%, 35%, 40%, 45%, 50%, 55%, 60%, 65%, 80%, 85%, 90%, 95%, or 100%, relative to tumor metabolism prior to administration of an AMO, as assessed by methods well known in the art, e.g., PET scanning. In particular embodiments, the methods for treating cancer provided herein inhibit or decrease tumor metabolism in a subject in the range of about 5% to 20%, 10% to 20%, 10% to 30%, 15% to 40%, 15% to 50%, 20% to 30%, 20% to 40%, 20% to 50%, 30% to 60%, 30% to 70%, 30% to 80%, 30% to 90%, 30% to 95%, 30% to 99%, 30% to 100%, or any range in between, relative to tumor metabolism prior to administration of an AMO, as assessed by methods well known in the art, e.g., PET scan.

Some embodiments provided herein describe methods of treating cancer, wherein the method comprises treating a patient with any one of the AMOs described herein. Some embodiments provided herein describe methods of treating cancer, wherein the method comprises treating a patient with vesicles containing one of the AMOs described herein. Some embodiments provided herein describe methods of treating cancer, wherein the method comprises treating a patient with cancer cell EVs obtained from the patient, wherein the cancer cell EVs obtained from the patient are contacted with or contain one of the AMOs described herein.

In some embodiments the present disclosure comprises a method of treating a neoplasia. In some embodiments, a neoplastic cell induces an inflammatory response. In some embodiments, part of the inflammatory response to a neoplastic cell is angiogenesis. In some embodiments, angiogenesis facilitates the development of a neoplasia.

In some embodiments, the methods described herein treat cancers such as lung, breast, brain, prostate, spleen, pancreatic, cervical, ovarian, head and neck, esophageal, liver, skin, kidney, leukemia, bone, testicular, colon, or bladder cancer. In some embodiments, the cancer is pancreatic cancer, colon cancer, breast cancer, T-cell leukemias, or lymphomas. In some embodiments, the cancer is leukemia, lymphoma, or multiple myeloma.

Solid tumor cancers that can be treated by the methods provided herein include, but are not limited to, sarcomas, carcinomas, and lymphomas. In specific embodiments, cancers that can be treated in accordance with the methods described include, but are not limited to, cancer of the breast, liver, neuroblastoma, head, neck, eye, mouth, throat, esophagus, esophagus, chest, bone, lung, kidney, colon, rectum or other gastrointestinal tract organs, stomach, spleen, skeletal muscle, subcutaneous tissue, prostate, breast, ovaries, testicles or other reproductive organs, skin, thyroid, blood, lymph nodes, kidney, liver, pancreas, and brain or central nervous system. In some embodiments, the solid tumors that can be treated by the methods provided herein include, but are not limited to, sarcomas, carcinomas, and lymphomas.

Also, provided herein are combination therapies for the treatment of cancer which involve the administration of an AMO in combination with one or more additional therapies to a subject in need thereof. In a specific embodiment, presented herein are combination therapies for the treatment of cancer which involve the administration of an effective amount of an AMO in combination with an effective amount of another therapy to a subject in need thereof.

In some embodiments, an AMO described herein is administered in combination with a chemotherapeutic agent. In some embodiments, the chemotherapeutic agent is cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, gemcitabien, navelbine, farnesyl-protein tansferase inhibitors, transplatinum, 5-fluorouracil, vincristin, Velcade, vinblastin, methotrexate, or any analog or derivative variant of the foregoing.

In some embodiments, an active agent described herein is administered in combination with radiotherapy. Radio therapy can include γ-rays, X-rays, and/or the directed delivery of radioisotopes to tumor cells. In certain embodiments, microwaves and/or UV-irradiation are used according to methods of the disclosure. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the methods for treating cancer provided herein comprise administering an AMO as a single agent for a period of time prior to administering an AMO in combination with an additional therapy. In some embodiments, the methods for treating cancer provided herein comprise administering an additional therapy alone for a period of time prior to administering an AMO in combination with the additional therapy.

In some embodiments, the administration of an AMO and one or more additional therapies in accordance with the methods presented herein have an additive effect relative the administration of an AMO or the one or more additional therapies alone. In some embodiments, the administration of an AMO and one or more additional therapies in accordance with the methods presented herein have a synergistic effect relative to the administration of an AMO or the one or more additional therapies alone.

The combination therapies provided herein involve administering to a subject to in need thereof an AMO in combination with conventional, or known, therapies for treating cancer. Other therapies for cancer or a condition associated therewith are aimed at controlling or relieving one or more symptoms. Accordingly, in some embodiments, the combination therapies provided herein involve administering to a subject to in need thereof a pain reliever, or other therapies aimed at alleviating or controlling one or more symptoms associated with or a condition associated therewith.

Specific examples of anti-cancer agents that may be used in combination with an AMO include: a hormonal agent (e.g., aromatase inhibitor, selective estrogen receptor modulator (SERM), and estrogen receptor antagonist), chemotherapeutic agent (e.g., microtubule disassembly blocker, antimetabolite, topoisomerase inhibitor, and DNA crosslinker or damaging agent), anti-angiogenic agent (e.g., VEGF antagonist, receptor antagonist, integrin antagonist, vascular targeting agent (VTA)/vascular disrupting agent (VDA)), radiation therapy, and conventional surgery.

Non-limiting examples of hormonal agents that may be used in combination with an AMO include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that may be used in combination with an AMO include microtubule disassembly blocker, antimetabolite, topisomerase inhibitor, and DNA crosslinker or damaging agent. Chemotherapeutic agents that are microtubule dissemby blockers include, but are not limited to, taxenes (e.g., paclitaxel (branded/marketed as TAXOL®), docetaxel, abraxane, larotaxel, ortataxel, and tesetaxel); epothilones (e.g., ixabepilone); and vinca alkaloids (e.g., vinorelbine, vinblastine, vindesine, and vincristine (branded/marketed as ONCOVIN®)).

Chemotherapeutic agents that are antimetabolites include, but are not limited to, folate anitmetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine (GEMZAR®), cytarabine, decitabine, floxuridine, tegafur); and deoxyribonucleotide antimetabolites (e.g., hydroxyurea).

Chemotherapeutic agents that are topoisomerase inhibitors include, but are not limited to, class I (camptotheca) topoisomerase inhibitors (e.g., topotecan (branded/marketed as HYCAMTIN®) irinotecan, rubitecan, and belotecan); class II (podophyllum) topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone, and pixantrone).

Chemotherapeutic agents that are DNA crosslinkers (or DNA damaging agents) include, but are not limited to, alkylating agents (e.g., cyclophosphamide, mechlorethamine, ifosfamide (branded/marketed as IFEX®), trofosfamide, chlorambucil, melphalan, prednimustine, bendamustine, uramustine, estramustine, carmustine (branded/marketed as BiCNU®), lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin (branded/marketed as PARAPLATIN®), cisplatin, oxaliplatin, nedaplatin, triplatin tetranitrate, satraplatin, picoplatin); nonclassical DNA crosslinkers (e.g., procarbazine, dacarbazine, temozolomide (branded/marketed as TEMODAR®), altretamine, mitobronitol); and intercalating agents (e.g., actinomycin, bleomycin, mitomycin, and plicamycin).

Non-limiting examples of other therapies that may be administered to a subject in combination with an AMO include: a statin; an mTOR inhibitor; a farnesyltransferase inhibitor agent; an antifibrotic agent; a pegylated interferon; a CNS stimulant; a HER-2 antagonist; an IGF-1 antagonist or an IGF-1 kinase inhibitor; EGFR/HER-1 antagonist or EGFR kinase inhibitor (SRC antagonist; cyclin dependent kinase (CDK) inhibitor; Janus kinase 2 inhibitor; proteasome inhibitor; phosphodiesterase inhibitor; inosine monophosphate dehydrogenase inhibitor; lipoxygenase inhibitor; endothelin antagonist; retinoid receptor antagonist; immune modulator; kinase inhibitor; non-steroidal anti-inflammatory agent; human granulocyte colony-stimulating factor (G-CSF); folinic acid or leucovorin calcium; integrin antagonist; nuclear factor kappa beta (NF-κβ) antagonist; hedgehog inhibitor; histone deacetylase (HDAC) inhibitor; retinoid; hepatocyte growth factor/scatter factor (HGF/SF) antagonist; synthetic chemical; anti-diabetic; antimalarial and amebicidal drug; synthetic bradykinin; platelet-derived growth factor receptor inhibitor; receptor tyrosine kinase inhibitors of Flk-1/KDR/VEGFR2, FGFR1 and PDGFR beta; anti-inflammatory agent; and TGF-beta antisense therapy.

### Pharmaceutical Compositions

In some embodiments, AMOs may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising AMOs encompass any pharmaceutically acceptable salts, esters, or salts of such esters. In some embodiments, pharmaceutical compositions comprising AMOs comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an AMO which are cleaved by endogenous nucleases within the body, to form the active AMO compound.

Lipid moieties have been used in nucleic acid therapies in a variety of methods. In certain such methods, the nucleic acid is introduced into preformed liposomes or lipoplexes made of mixtures of cationic lipids and neutral lipids. In certain methods, DNA complexes with mono- or poly-cationic lipids are formed without the presence of a neutral lipid. In some embodiments, a lipid moiety is selected to increase distribution of a pharmaceutical agent to a particular cell or tissue.

In some embodiments, pharmaceutical compositions provided herein comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In some embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In some embodiments, certain organic solvents such as dimethylsulfoxide are used.

Pharmaceutical compositions containing target polynucleotide inhibitors suitable for use in the methods of the present invention can include a pharmaceutically acceptable carrier as described infra, one or more active agents, and a suitable delivery vehicle. Suitable delivery vehicles include, but are not limited to viruses, bacteria, biodegradable microspheres, microparticles, nanoparticles, liposomes, collagen minipellets, and cochleates. In one embodiment of the present invention, the pharmaceutical composition or formulation containing an inhibitory oligonucleotide, e.g., an AMO is encapsulated in a lipid formulation to form a nucleic acid-lipid particle.

Pharmaceutical compositions and formulations can involve incorporation of AMOs within a variety of macromolecular assemblies, micelle, or liposome compositions for cellular delivery. In some embodiments, the AMOs are formulated for conventional intravenous, subcutaneous, or intramuscular dosing. Such formulations may be conventional aqueous preparations, such as formulation in saline. In some embodiments, the AMOs are suitable or formulated for intradermal, subcutaneous, intramuscular, intraperitoneal or intravenous injection, or by direct injection into target tissue (e.g., tumor tissue). In some embodiments, a pharmaceutical composition provided herein is prepared for oral administration. In some embodiments, pharmaceutical compositions are prepared for buccal administration.

In still other aspects, the invention provides a method for delivering AMOs and the pharmaceutical compositions to mammalian cells either in vitro or ex vivo, e.g., for treating, ameliorating, or preventing the progression of a condition in a mammalian patient. The method may comprise administering the AMO to a mammalian patient or population of target cells. The patient may have a condition associated with, mediated by, or resulting from, hY5 fragment generation in cancer cell EVs. Such conditions include, for example, cancer. Thus, the invention provides a use of the modified oligonucleotides and compositions of the invention for treating such conditions, and for the preparation of medicaments for such treatments.

In some embodiments, a pharmaceutical composition provided herein comprises one or more tissue-specific delivery molecules designed to deliver the one or more pharmaceutical agents of the present invention to specific tissues or cell types. For example, in certain embodiments, pharmaceutical compositions comprising AMOs include vesicles coated with a tissue-specific antibody.

### Subjects

The term “non-human animals” and “non-human mammals” includes all vertebrates, e g, mammals, such as non-human primates, (particularly higher primates), sheep, dog, rodent (e.g. mouse or rat), guinea pig, goat, pig, cat, rabbits, and cows. In one embodiment, the subject is human. In another embodiment, the subject is an experimental animal or animal substitute as a disease model. “Mammal” refers to any animal classified as a mammal, including humans, non-human primates, domestic and farm animals, and zoo, sports, or pet animals, such as dogs, cats, cattle, horses, sheep, pigs, goats, rabbits, etc. Patient or subject includes any subset of the foregoing, e.g., all of the above, but excluding one or more groups or species such as humans, primates or rodents. A subject can be male or female. A subject can be a fully developed subject (e.g., an adult) or a subject undergoing the developmental process (e.g., a child, infant or fetus). In some embodiments, the compositions provided herein are administered to a cell ex vivo.

### Computer Systems

Certain methods described herein can be implemented by one or more computer systems or can include or be implemented in software comprising machine-executable code, which can run on such computer systems or other systems. For example, the software can be executable by a computer system, for example, that functions as the storage server or proxy server, and/or that functions as a user's terminal device.

In some cases, software can be stored on a computer system in the form of a non-transitory computer readable medium. The non-transitory computer readable medium can have stored therein sequences of instructions which, when executed by a computer system, cause the computer to perform methods described herein. Computer readable medium are well known in the art and described, e.g., in U.S. Pat. No. 7,783,072.

A computer can be in communication with a device, e.g., a thermocycler or a device for performing PCR. A computer can be connected to the Internet through a wired or wireless connection. In some cases, a health care provider or subject sends a sample to a service provider that analyzes the sample using the methods, compositions, or kits described herein. In some cases, a computer is used to transmit results of a reaction to a subject. In some cases, the subject is a patient. In some cases, a computer is used to transmit results of a reaction to a healthcare provider, e.g., a physician, or to an insurance company. In some cases, a computer is used to generate a report comprising results of one or more tests and/or additional assays.

### EXAMPLES

### Example 1

### Isolation of Extracellular Vesicles (EVs)

K562 cells were grown in complete RPMI-1640 medium (10% FBS+1% Penicillin-Streptomycin) and BJ cells were grown in DMEM (10% FBS and 1% penicillin-streptomycin). When the cells reached approximately 70-80% confluence, the media was replaced with serum-free conditioned medium and incubated for another 24 hr. The Conditioned medium was then centrifuged at 300 g for 10 min. The cell pellet was discarded and the supernatant was further centrifuged at 2000 g for 10 min The Pellet, comprising of mostly cell debris and apoptotic bodies was discarded and the supernatant was again centrifuged at 10000 g for 30 min. The pellet, comprising of microvesicles was discarded and the supernatant was filtered at 3500 g for 15 min using Centricon Plus70 100 KD NMWL cut-off (Millipore). The filtrate was discarded and the residue, enriched with EVs and other proteins was collected. The collected residues were precipitated overnight using ExoQuick-TC (System Biosciences) at 1:5 ratio (by volume) of ExoQuick to filtration residue. Next morning, the sample was centrifuged at 1500 g for 30 min. The supernatant was discarded and the pellet was centrifuged again at 1500 g for 5 min Left over supernatant, if any, was discarded and the pellet and re-suspended in 500 μL PBS.

### Example 2

### Electron Microscopy

Negative staining of exosome suspensions followed by imaging in a transmission electron microscope was used to determine vesicle shape and size distribution. Aliquots of exosome suspensions were dispensed onto sheets of Parafilm in a humidified petri dish and the vesicles were adsorbed onto freshly prepared Butvar coated EM grids (glow discharged). The adsorption was done for 5 min at room temperature. The petri dish containing the suspensions and EM grids was transferred to a large bucket of ice shavings and the grids were transferred to three successive drops of distilled water (30 s each) to remove salts, and then transferred to a drop of 1% uranyl acetate in 1% methyl cellulose for 30 s and then placed in a second drop of negative stain solution for 5 min. Excess stain was blotted off and the grids were air dried.

Immuno-gold labeling for the CD81 was done by re-suspending the EVs in primary mouse antibody to human CD81 (Abcam) diluted in PBS for 30 min at room temperature. Incubations were done in sterile 1.5 mL microcentrifuge tubes. The antibody labeled vesicles were pelleted by centrifugation, re-suspended in a 1:10 solution of 5 nm colloidal gold conjugated to rabbit anti-mouse IgM secondary antibody (Aurion, Electron Microscopy Sciences) for 30 min. The gold labeled vesicles were then adsorbed to Butvar-coated grids for 5 min and then rinsed through 3 drops of PBS to remove unbound gold particles. Negative staining of the gold labeled vesicles was completed at described above. Samples were imaged in the Hitachi H7000 Electron Microscope operated at 75 kV. Images recorded on Kodak EM film 4489 were scanned at 2400 DPI on an Epson Perfection V750 film scanner.

### Example 3

### Western Blot

Proteins were isolated using RIPA buffer (Pierce) using manufacturer's protocol, concentrated using Amicon Ultra 3K centrifugal filter (Millipore) and quantified using BCA protein quantification kit (Pierce). 1 microgram of proteins from K562 whole cell and EVs were loaded on pre-cast 4-20% Tris-Glycine gel and transferred to PVDF membrane. Membrane was blocked using Pierce TBST blocking buffer for 1 hr at room temperature (RT). Primary antibody incubation was performed overnight at 4° C. at a 1:1000 dilutions while secondary antibodies were used at 1:10,000 dilutions. Membranes were developed with Amersham ECL plus western blotting development kit (GE). Anti-Fibrillarin, Anti-Protein disulphide Isomerase antibodies and Anti-Prohibitin were used as nuclear, endoplasmic reticulum and mitochondrial marker, respectively. Anti-PDC6I, Anti-Tsg101 and Anti-Transferrin receptor antibodies were used as exosomal marker. Goat polyclonal antibody to Rabbit IgG and Rabbit polyclonal antibody to Mouse IgG were used as secondary antibodies.

### Example 4

### Nanoparticle Tracking Analysis

Quantification of the extracellular vesicles was performed by Nanoparticle Tracking Analysis (NTA) was performed using NanoSight LM10 at 25° C. PBS was used as a diluent and samples were run at 1:500 dilutions for K562 EVs and 1:5 dilutions for BJ EVs

### Example 5

### Isolation of RNA

RNA isolation was performed using an Ambion Mirvana miRNA Isolation kit Prior to RNA isolation, EVs were treated with Ambion RNAse cocktail at 37° C. for 15 min. 1 mL of lysis/binding buffer was immediately added to the RNAse treated EVs to deactivate the RNAse.

Alternatively, RNA purification was performed with Trizol LS (Invitrogen, Life Technologies). In some fractions, 10 g RNAse-free glycogen was added as a carrier. RNA was quantified using a Qubit 2.0 fluorometer (Life Technologies) and a Qubit RNA high sensitivity kit, according to manufacturer's instructions.

### Example 6

### Detergent and RNAse Treatment

To determine if the isolated RNA were exosomal RNA cargo and not any artifact of purification, RNA isolated from EVs without RNAse treatment was compared with RNA isolated from RNAse treated EVs and RNA isolated from detergent and RNAse treated EVs. RNAse treatment of EVs re-suspended in PBS was performed with Ambion RNAse cocktail at 37° C. for 15 min Detergent treatment was performed with RIPA buffer for 15 min followed by RNAse treatment as described above

### Example 7

### Small RNA Sequencing

Small RNA was isolated with Mirvana miRNA isolation kit (Life Tech) and DNase treated with Ambion Turbo-DNase (Life Tech). Ribosomal RNA depletion was performed on Whole cell RNA using Eukaryote Ribominus kit (Life Tech) using manufacturer's protocol. Both exosomal and whole cell RNA was treated with Tobacco Acid Pyro-phosphatase (Epicenter) to make 5′ capped and tri-phosphate RNAs amenable to adapter ligation. Libraries were constructed using Illumina TruSeq small RNA kit according to manufacturer's protocol, except reverse transcription was 200nts region was cut and gel-purified with Qiagen gel extraction kit. Libraries were quantified on Agilent Bio-analyzer HS-DNA chip and sequenced on Illumina HiSeq2000.

### Example 8

### Bioinformatics Analysis

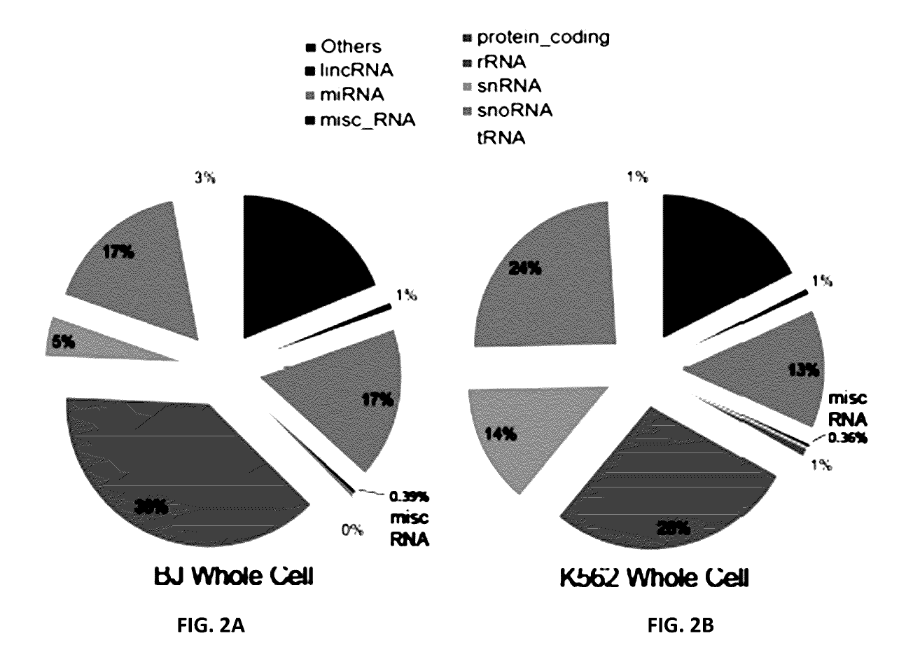

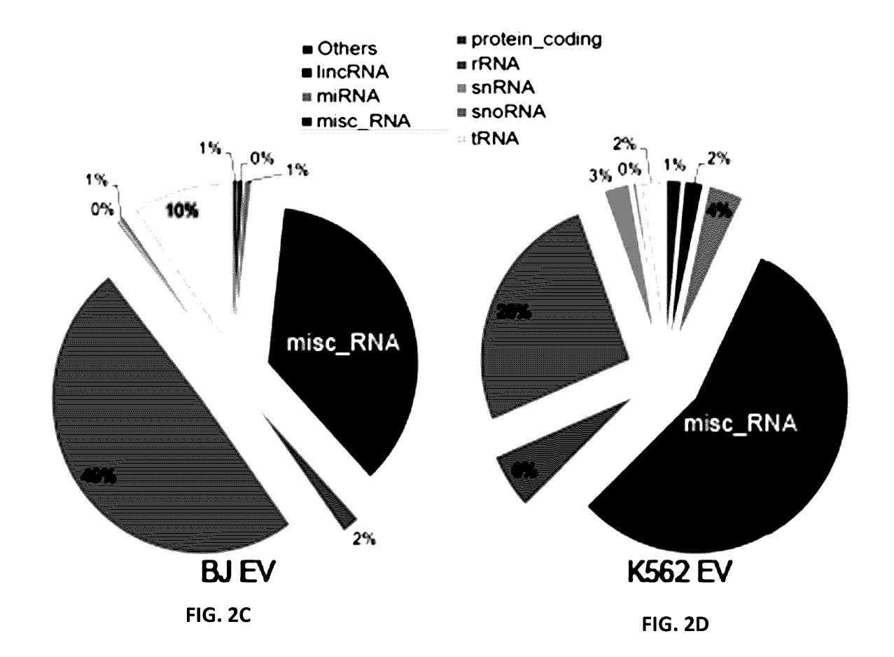

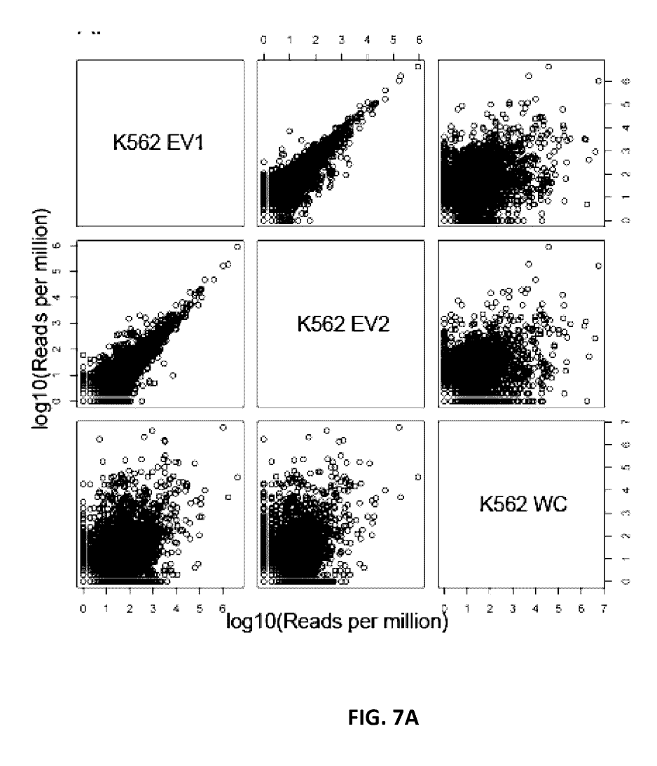

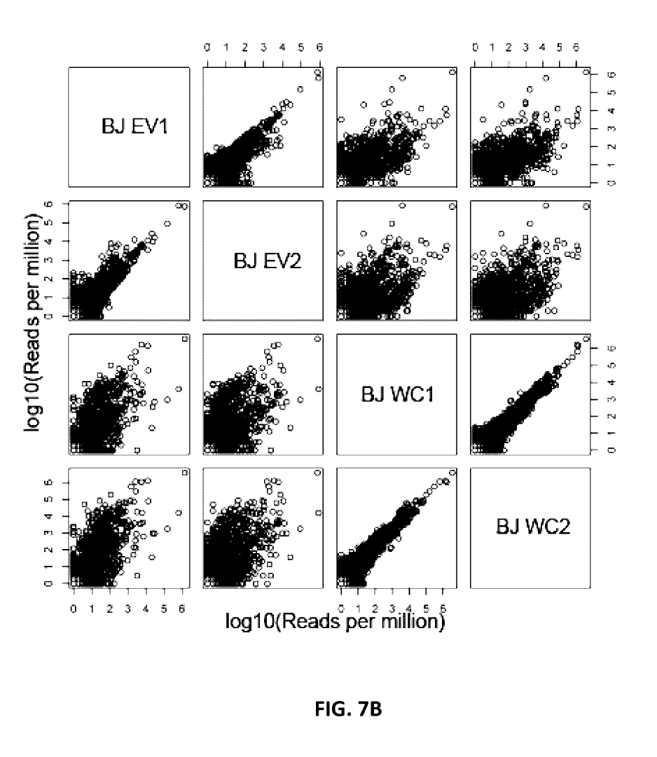

All data from RNA sequencing experiments in the study were mapped to Human Genome version 19 (hg19, GRCh37) obtained from the UCSC genome browser website. RNAseq reads were aligned using the STAR v1.9 software, and up to 5 mismatches per alignment were allowed. Only alignments for reads mapping to 10 or fewer loci were reported. Annotations were not utilized for mapping the data. The obtained BAM files were further processed using HTSeq software in order to appropriate the number of reads originating from each annotated regions of the genome, utilizing annotations obtained from Gencode v19 of the human genome, using the “Union mode” option of the software for all libraries, tRNA annotations were obtained from tRNAscan database. Reads per million (rpm) values for each gene was obtained by dividing the number of reads uniquely mapping within the limits of a gene annotation, by the total number of uniquely mapping reads in the library and multiplying by a million. These rpm values were used between replicates (FIG. 7A, B) to establish correlation between biological replicates of exosomal RNA libraries. Relative abundance of RNA families (FIG. 2A-D), was calculated using the cumulative rpm values of all genes within the Gencode defined RNA families such as miRNA, snoRNA, miscellaneous RNA (miscRNA), protein coding etc. Within each pie chart in FIG. 2, the group termed as “Others” includes Gencode all categories other than lincRNA, miRNA, miscRNA, rRNA, tRNA, snRNA, snoRNA and protein coding genes, (such as 3′-overlapping-ncRNA, immune-globulin genes, mitochondrial tRNA, mitochondrial rRNA, anti-sense RNA, antisense, pseudogenes, T-cell receptor genes, sense-intronic, sense-overlapping genes, etc). Density plots, were obtained by calculating the ratio of rpm within exosomes to the sum of rpms within exosomes and whole cell for both K562 and BJ cells (FIG.). The density function for genes of each RNA family within these graphs was calculated from these ratios using the kernel density function within the R stats package.

Fragment analysis to identify the most commonly found fragments within the hY5 gene was found by taking into account start and end positions of all reads that mapped to the hY5 gene from chromosome 7 between position 148638580 and 148638658 in the positive strand. All reads which began at the 5′ end of hY5 gene and were greater than 29nt in length mapped uniquely to hY5 gene. Similarly reads that began in places other than the 5′ end of the hY5 gene mapped uniquely to the genes primary location on chromosome 7. However genes which started in the 5′ end of the gene and were 29nt in length or shorter were all multi-mappers and mapped with 100% identity to two other locations (chromosome 12:45581224-45581252 and chromosome 13:103472349-103472369) and 97% identity to few other locations (chromosome 12:98223788-98223816, chromosome 19:36540048-36540076, and chromosome 1:35893466-35893493), thus making it impossible to accurately establish the true origin of these reads absolutely. These locations are annotated as pseudogenes of the hY5 gene, and to resolve this uncertainty of their origin they were included for the fragment analysis. The secondary structure of hY5 was obtained using the online resource of the mfold package, within which the most frequently occurring fragments were highlighted.

In order to identify genes which are differentially expressed (DE) between time points for the molecular phenotype section, bio-replicates from time points 2, 6, and 24 hr after treatment with exosomes were compared to the untreated replicates, by using DESeq on the read counts of the genes derived from the HTSeq software, filtering by false discovery rate (FDR) less than 0.01 and by fold-change greater than or equal to 2 or less than or equal to 0.5. The fold change at the time point of maximal change was then taken into account as the maximal amplitude of change for each gene. The list of DE genes common between the two cell types on treatment with K562 exosomes and the list of DE genes common between the two cell types after 5′ 32-mer treatment were then used for further over-representation analysis on the GO biological processes using the online resource of Panther Pathways, where only biological processes with a p-value less than 0.05 was taken to be significant. The list and map of genes within the FAS/TGF-β pathway was obtained from KEGG pathways and those genes within out DE gene lists were overlaid on the map, where red color indicates a fold change below 0.05, and green indicates fold change greater than 2, and blue indicates no significant fold change after treatment in each cell type.

### Example 9

### Lipid Labeling of EVs & Imaging

K562 EVs were isolated as described above. 2 microliter of PKH67 (Sigma, cat. no. MINI67-1KT) was re-suspended in 500 μL diluent and added to purified EVs for 4 min in dark and EVs were isolated using ExoQuick-TC as described above. The labelled exosomal pellet was re-suspended in complete medium (DMEM+10% FBS+1% Penicillin-Streptomycin) and added to BJ cells for overnight incubation. Imaging was done on Deltavision OMX microscope and image analysis was performed with Delta-vision SoftWorx software.

### Example 10

### Metabolic Labeling of RNA & Imaging

K562 cells (2×10⁷) were incubated at a final concentration of 0.2 mM 5-Ethnyl uridine (EU) for 24 hr. EVs were isolated from the conditioned medium as described above. 3T3 cells were treated with ActinomycinD at a final concentration of 1 μM for 1 hr to block its endogenous transcription. The drug-treated media was replaced with fresh complete DMEM medium and the cells were incubated with EU labeled K562 EVs for 2 hr. The cells were subsequently fixed with 4% Para-formaldehyde and permeabilized with 0.5% Triton-X-100. EU incorporated exosomal RNA was detected using Click chemistry and nuclei was counterstained using Hoechst. Cells were imaged on Delta-vision OMX microscope and image analysis was performed with Delta-vision SoftWorx. As a negative control, 3T3 cells treated with ActinomycinD and directly incubated with EU was performed which showed no signal of EU-incorporated RNA thus confirming block of endogenous transcription

### Example 11

### Subcellular Localization of hY5 31-Mer

2×10⁵ BJ cells were plated overnight and next morning cells were transfected with 100 pmol of synthetic hY5 31-mer coupled with Alexa 488 fluorophore at its 3′ end. After 6 hr, transfection medium (Opti-MEM) was replaced with complete DMEM medium and incubated for another 24 hr. Imaging was performed on Delta-Vision OMX microscope and Image processing was performed with Delta-vision SoftWorx software.

### Example 12

### Interspecies Transfer of RNA by RNASeq

Mouse HB4 cells (ATCC) were treated with K562 EVs for 0 hr, 12 hr, and 24 hr and HB4 cells untreated (Neg. control) and RNA isolation was performed using Mirvana miRNA isolation kit. Isolated RNA was ethanol-precipitated, DNase treated and size separated into long (>200nt) and short RNA (<200nt). The short RNA was ribo-depleted using Ribo-minus Eukaryote ribo-depletion kit (Life Tech) using manufacturer's protocol and ethanol precipitated.

The precipitated RNA was then treated with Tobacco Acid Pyrophosphatase at 37° C. for 1 hr to convert the 5′ capped and triphosphate RNA molecules into 5′ monophosphate and make them amenable for adapter ligation. RNA was then purified by phenol-chloroform treatment followed by ethanol precipitation. The Small RNA libraries were then constructed using a-tailing protocol. The amplified libraries were then run on 2% agarose gel and the region between 20-200nt was cut and gel extracted with Qiagen gel extraction kit. Finally, libraries were quantified using Agilent Bioanalyzer and sequenced on Illumina MiSeq platform. Mapping was performed by STAR against combined Human and Mouse genome and reads which mapped uniquely to humans only were considered for analysis. hY5, a human specific gene enriched in EVs was used as a marker to demonstrate inter-species transfer of human K562 EV RNA to Mouse HB4 cells.

### Example 13

### Oligonucleotide End-Labeling

Oligonucleotides (90 pmol for DNA oligonucleotides and 15 pmol for RNA oligonucleotides) were end-labeled in reactions containing 20 μCi of γ-³²P-ATP (PerkinElmer), 5 units T4 polynucleotide kinase (New England BioLabs), 70 mM Tris-HCl pH 7.6, 10 mM MgCl₂, and 5 mM dithiothreitol (DTT). Labeling proceeded for 30 min at 37° C., followed by phenol-chloroform extraction.

### Example 14

### Northern Blots

Whole cell total RNA and EV RNA from K562 and BJ cells (850 ng each) was separated on 8% acrylamide, 8 M urea gels. Thereafter, the RNA was blotted to nitrocellulose membranes (Zeta-Probe, Bio-Rad). The blots were probed with an oligonucleotide complementary to the 5′ end of the hY5 transcript (5′-CTT AAC AAT AAC CCA CAA CAC TCG GAC CAA CT-3′) (SEQ ID NO:37).

### Example 15

### In Vitro Processing

K562 Whole cell and EV proteins were extracted with RIPA buffer (Thermo Scientific). Cold processing reactions contained the indicated amount of protein, 10 mM MgCl₂, 10 mM DTT and 2 pmol synthetic full length hY5 RNA where indicated. After 30 min incubation at 37° C., reactions were phenol-chloroform extracted, separated on 8% acrylamide, 8 M urea gels, then blotted and probed as described for northern blots. Hot processing reactions were performed with synthetic versions of wild type hY5 5′ 31-mer (SEQ ID NO:38), shuffled 31-mer (5′-UGG UGC GUG UUG UUU AGA UUA AGU GGU UGA C-3′) (SEQ ID NO:40) or hY5 31-mer with a core8nt motif shuffled (GUU GUG GG (SEQ ID NO: 1)→ACG UAC AG) (SEQ ID NO:42). Each reaction contained 4 μg of K562 EV protein extract where indicated, 10 mM MgCl₂ and 0.15 pmol of end labeled RNA. After 2 hr incubation at 37° C., samples were separated on 8% acrylamide, 8 M urea gels. Thereafter, the gels were subjected to autoradiography.

### Example 16

### RNA Transfection

2×10⁵ cells were plated in 6-well plates overnight. The next day, RNA transfection was performed with Lipofectamine 2000 and Opti-MEM medium for 6 hr. After 6 hr, Opti-MEM media was replaced with complete medium and cells were incubated for another 24 hr.

### Example 17

### Flow Cytometry

Quantification of cell death was performed on a BD LSR-II Cell Analyzer (BD Biosciences, San Jose, Calif.) using a flow cytometry kit that detects membrane permeability, chromatin condensation and dead cell apoptosis (Life Tech, cat. no. V23201). YO-PRO-1 was excited by the 488 nm laser and its emission was collected with a 530/30 filter. A 405 nm Violet laser was used to excite Hoechst and emission was collected with a 440/40 filter. Unstained cells and single color control samples (YO-PRO-1 only and Hoechst only) were used for setting the PMT voltages and eliminating any spectral overlap between these two fluorochromes. Only events positively labeled with Hoechst were considered for quantification. Cells double-labeled with Hoechst and Yo-Pro-1 were quantified as “dead cells” and cells labeled with Hoechst but not with Yo-Pro-1 was quantified as “living cells”. YO-PRO1, a nucleic acid binding dye which was permeable to apoptotic and dead cells but not living cells was used for quantification of cell death. Cells were trypsinized and re-suspended in 800 μL DMEM medium. Cells were labeled with 1 microliter of YO-PRO1 and Hoechst for 15 min at room temperature. The labeled cells were kept on ice and then passed through a cell strainer prior to running on the LSR-II.

### Example 18

### EVs Incubation with Cells and Cell Death Quantification

EVs were isolated from 1×10⁸ cancer (K562, HeLa, U205, and MCF7) or primary (BJ) cells as explained above and incubated with BJ or K562 cells for 24 hr. After 24 hr, quantification of cell death was performed by flow cytometry as explained above. Percent of cell death observed in K562 cells when treated with K562 EV and EV RNA was shown in Table 1A. Percent of cell death observed in BJ cells when treated with cancer and primary EV and EV RNA was shown in Table 1B.

TABLE 1A

| Sample | Rep1 | Rep2 | Mean |
|---|---|---|---|
| Untreated | 2.4 | 3.5 | 2.95 |
| K562 EV treated | 6.6 | 4.2 | 5.4 |
| MOCK treated | 4.4 | 4.3 | 4.35 |
| K562 EV RNA treated | 4.2 | 4 | 4.1 |
| Complete scrambled 31-mer treated | 3.5 | 4.5 | 4 |

TABLE 1B

| Sample | Rep1 | Rep2 | Mean |
|---|---|---|---|
| Untreated | 4 | 4.9 | 4.45 |
| Mock treated | 7.1 | 7.7 | 7.4 |
| BJ EV RNA treated | 10.3 | 10.9 | 10.6 |
| K562 EV RNA treated | 20 | 21 | 20.5 |
| HELA EV treated | 26.8 | 18.52 | 22.66 |
| U2OS EV treated | 26.9 | 11.36 | 19.13 |
| MCF7 EV treated | 35.45 | 16.9 | 26.175 |
| K562 EV treated | 27.8 | 21.9 | 24.85 |
| BJ EV treated | 5.4 | 4.7 | 5.05 |

### Example 19

### Exosomal RNA Transfection and Quantification of Cell Death

Exosomal RNA was isolated from K562 and BJ EVs in duplicates with Mirvana miRNA isolation kit as explained above. RNA transfection was performed with Lipofectamine-2000 and Cell death quantification was performed after 24 hr incubation by flow cytometry as described above. Net increase in cell death with 100 pmol of hY5 31-mer treatment of cancer and primary cells (hY5 treatment—mock) was shown in Table 1C. Dose response of hY5 31-mer (percent cell death) and nonspecific RNA control in BJ cells was shown in Table 1D.

TABLE 1C

| Cells | Rep1 | Rep2 | Mean |
|---|---|---|---|
| BJ | 17.25 | 16.85 | 17.05 |
| IMR90 | 8.9 | 9.4 | 9.15 |
| HUVEC | 14.7 | 13.5 | 14.1 |
| HFFF (200 pmol hY5 31-mer) | 13.6 | 13.4 | 13.5 |
| MCF7 | 0 | 0 | 0 |
| HeLa | 8.15 | 7.1 | 7.625 |
| U2OS | 0.75 | 2.75 | 1.75 |
| K562 | 0 | 2 | 1 |

TABLE 1D

| Sample | Rep1 | Rep2 | Mean |
|---|---|---|---|
| Untreated | 1.3 | 1.6 | 1.45 |
| Mock treated | 2.6 | 2.5 | 2.55 |
| Nonspecififc RNA 10 pmol | 5.1 | 4.9 | 5 |
| Nonspecific RNA 50 pmol | 5.5 | 5.5 | 5.5 |
| Nonspecific RNA 100 pmol | 5.8 | 5.8 | 5.8 |
| Nonspecific RNA 200 pmol | 8.3 | 8.2 | 8.25 |
| Nonspecific RNA 300 pmol | 6.2 | 6.5 | 6.35 |
| Nonspecific RNA 400 pmol | 11.2 | 10.6 | 10.9 |
| hY5 31-mer10 pmol | 6.4 | 6.5 | 6.45 |
| hY5 31-mer 50 pmol | 8.8 | 9.1 | 8.95 |
| hY5 31-mer 100 pmol | 12.2 | 12 | 12.1 |
| hY5 31-mer 200 pmol | 23.8 | 22.3 | 23.05 |
| hY5 31-mer 300 pmol | 30 | 29.6 | 29.8 |
| hY5 31-mer 400 pmol | 40.9 | 40.5 | 40.7 |

### Example 20

### Synthetic Ribonucleotides Transfection and Cell Death Quantification

2×10⁵ BJ or K562 cells were plated overnight and next day, cells were transfected with 100 pmol of hY5 31-mer and 100 pmol hY5 23-mer with 5 μL Lipofectamine-2000 in Opti-MEM medium. After 6 hr, Opti-MEM media was replaced with complete DMEM media (for BJ) or complete RPMI-1640 medium (for K562). Untreated and Mock treatment was used as negative controls. AllStars negative control siRNA was used as non-specific RNA control. A 31nt scrambled RNA oligonucleotide was used as a scrambled RNA control. Furthermore, RNA oligonucleotides with 8nt motif (nucleotides 14-21) scrambled, scrambled with secondary structure intact and 8nt motif deleted oligonucleotide were used as controls for identifying the motif sequence responsible for phenotype. Finally, transfection of 83nt full length hY5 and a double stranded hY5 31-mer shows substantially lower cell death. Percent cell death in BJ cells with synthetic hY5 31-mer and controls was shown in Table 1E. Table 1F shows % K562 cell death with synthetic hY5 31-mer and controls.

TABLE 1E

| Sample | Rep1 | Rep2 | Mean |
|---|---|---|---|
| Untreated | 1.45 | 2.5 | 1.97 |
| Mock treated | 2.75 | 4 | 3.37 |
| Allstar nonspecific RNA control | 6 | 5.8 | 5.9 |
| 8 nucleotide motif deleted | 5.3 | 4.8 | 5.05 |
| Complementary side 32-mer | 4.2 | 4.5 | 4.35 |
| 8 nucleotide motif scrambled | 7.1 | 7.4 | 7.25 |
| hY5 completely scrambled 31-mer | 6.6 | 7.9 | 7.25 |
| Double stranded hY5 31-mer | 9.3 | 9.3 | 9.3 |
| Full length hY5 | 10.5 | 11 | 10.75 |
| hY5 31-mer | 19.8 | 17.43 | 18.61 |
| hY5 23-mer | 25.8 | 26.4 | 26.1 |

TABLE 1F

| Sample | Rep1 | Rep2 | Mean |
|---|---|---|---|
| Untreated | 9.2 | 10.5 | 9.85 |
| Mock | 9.7 | 10.5 | 10.1 |
| 8nt motif deleted | 11.9 | 10.5 | 11.2 |
| 8nt motif scrambled | 10.3 | 10.2 | 10.25 |
| Allstar nonspecific RNA treated | 11.6 | 9.7 | 10.65 |
| 31-mer scrambled | 8.9 | 8.8 | 8.85 |
| Full length hY5 | 9.8 | 9.5 | 9.65 |
| Double stranded hY5 31-mer | 10.3 | 10.3 | 10.3 |
| hY5 31-mer | 9.8 | 12.1 | 10.95 |

### Example 21

### Generality of the Phenotype

Generality of hY5 31-mer mediated cell death phenotype was assessed in 4 cancer (K562, HeLa, U2OS and MCF7) and 4 primary cells (BJ, HUVEC, IMR90 and Human fetal foreskin fibroblast (HFFF)). In each case, 2×10⁵ cells were plated overnight. Next day, cells were transfected with 100 pmo of synthetic hY5 31-mer (except HFFF, which was transfected with 200 pmol of hY5) and 5 μL Lipofectamine-2000 as described above. Cell death quantification was performed after 24 hr incubation as described above.

### Example 22

### Dose Response Curve of hY5 31-Mer

Transfection of BJ cells was performed with hY5 31-mer and Qiagen AllStars negative control siRNA (non-specific RNA control) in a dose dependent manner Briefly, 2×10⁵ cells were plated overnight and on the following day, cells were transfected with hY5 31-mer (10, 50, 100, 200, 300 and 400 pmol) or AllStars control (10, 50, 100, 200, 300 and 400 pmol) with 10 μL Lipofectamine in Opti-MEM medium. Both Untreated and Mock treated (Lipofectamine only) was also performed as negative controls. After 6 hr, media was replaced with complete DMEM medium and incubated for another 24 hr. Quantification of cell death was performed as described above

### Example 23

### Co-Culture and Cell Death Quantification

Co-culture of K562 and BJ cells were performed both as direct co-culture as well as transwell co-culture. In direct co-culture system, 2×10⁵ BJ cells were plated on 6 well plates and next day, cells were labeled with Hoechst33342 for 15 min in dark at 37° C. Cells were washed with thrice with PBS and replaced with complete DMEM medium. 2×10⁵K562 cells re-suspended in 2 mL RPMI-1640 medium were added to the same well and directly co-cultured with BJ cells. As negative control, BJ cells were grown alone in 2 mL DMEM+2 mL RPMI-1640 medium. After 24 hr, both cells were harvested together but were only labeled with YO-PRO-1. Quantification of cell death was performed by flow cytometry as described above. Since K562 cells, although present in the solution were not labeled with Hoechst, Hoechst and YO-PRO-1double labeled cells were quantified as “dead BJ cells” while Hoechst positive but YO-PRO-1negative cells were quantified as “living BJ cells”.

In Transwell co-culture system, 2×10⁵ BJ cells were plated at the bottom of the well. Next day, 2×10⁵ K562 cells were plated in RPMI medium in the same well but across a Transwell membrane (Corning, 1 μm pore size). After 24 hr, K562 cells on top of the membrane were discarded while the BJ cells on the well were labeled with YO-PRO-1 and Hoechst and flow cytometry was performed for quantification as described above.

### Example 24

### Synthetic RNA Oligonucleotides Sequences

Synthetic oligonucleotides used for this study include those depicted in Table 2 below.

TABLE 2

| | SEQ | |
|---|---|---|
| Synthetic Oligo | ID | Sequence (5′ to 3′) |
| hY5 31-mer: | 38 | rArGrUrUrGrGrUrCrCrGrArGrUrGrUrUrGrUrGrGrGrUrUrArUrUrGrUrUrArA |
| hY5 23-mer: | 39 | rArGrUrUrGrGrUrCrCrGrArGrUrGrUrUrGrUrGrGrGrUrU |
| hY5 31nucleotide | 40 | rUrGrGrUrGrCrGrUrGrUrUrGrUrUrUrArGrArUrUrArArGrUrGrGrUrUrGrArC |
| complete | | |
| scrambled: | | |
| hY5 8nucleotide | 41 | rArGrUrUrGrGrUrCrCrGrArGrUrUrUrArUrUrGrUrUrArA |
| motif deleted: | | |
| hY5 31-mer with 8 | 42 | 5rArGrUrUrGrGrUrCrCrGrArGrUrArCrGrUrArCrArGrUrUrArUrUrGrUrUrAr |
| nucleotide motif | | A |
| scrambled: | | |
| hY5 32-mer | 43 | rCrCrCrCrArCrArArCrCrGrCrGrCrUrUrGrArCrUrArGrCrUrUrGrCrUrGrUrUr |
| complementary (3′ | | U |
| side) fragment: | | |
| Full length hY5 | 44 | rArGrUrUrGrGrUrCrCrGrArGrUrGrUrUrGrUrGrGrGrUrUrArUrUrGrUrUrArAr |
| 83-mer: | | GrUrUrGrArUrUrUrArArCrArUrUrGrUrCrUrCrCrCrCrCrCrArCrArArCrCrGrC |
| | | rGrCrUrUrGrArCrUrArGrCrUrUrGrCrUrGrUrUrU |
| Double-stranded | 45 | rArGrUrUrGrGrUrCrCrGrArGrUrGrUrUrGrUrGrGrGrUrUrArUrUrGrUrUrArAr |
| hY5 31-mer: | | G |
| | 46 | rCrCrCrCrArCrArArCrCrGrCrGrCrUrUrGrArCrUrArGrCrUrUrGrCrUrGrUrUr |
| | | U |

### Example 25

### Isolation, Quantification and Characterization of EV RNA Cargoes of Primary and Cancer Cell Lines

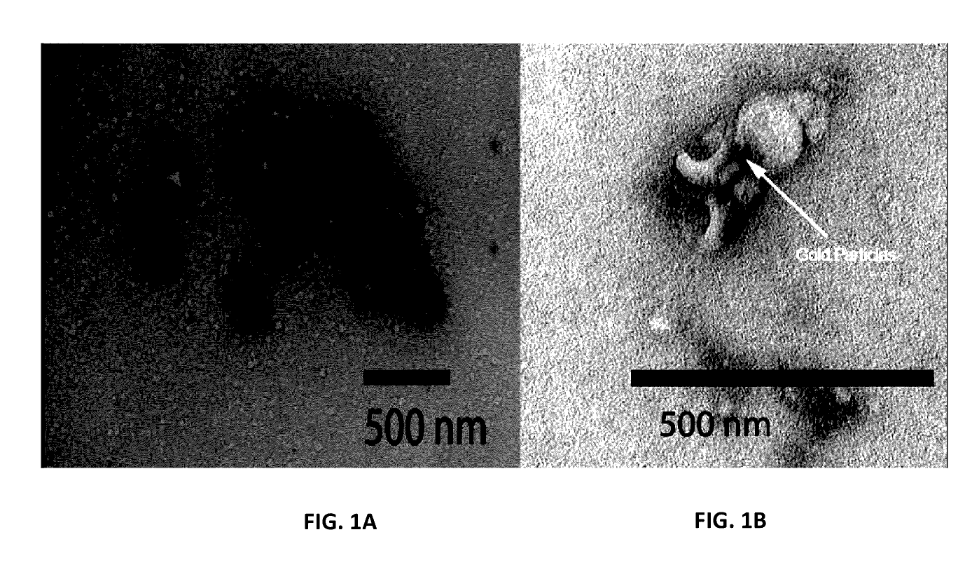

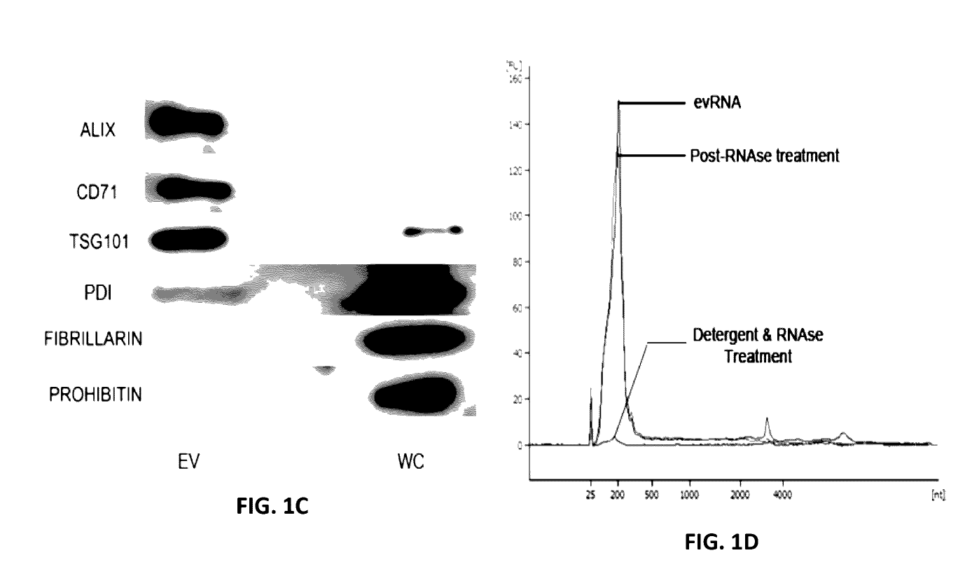

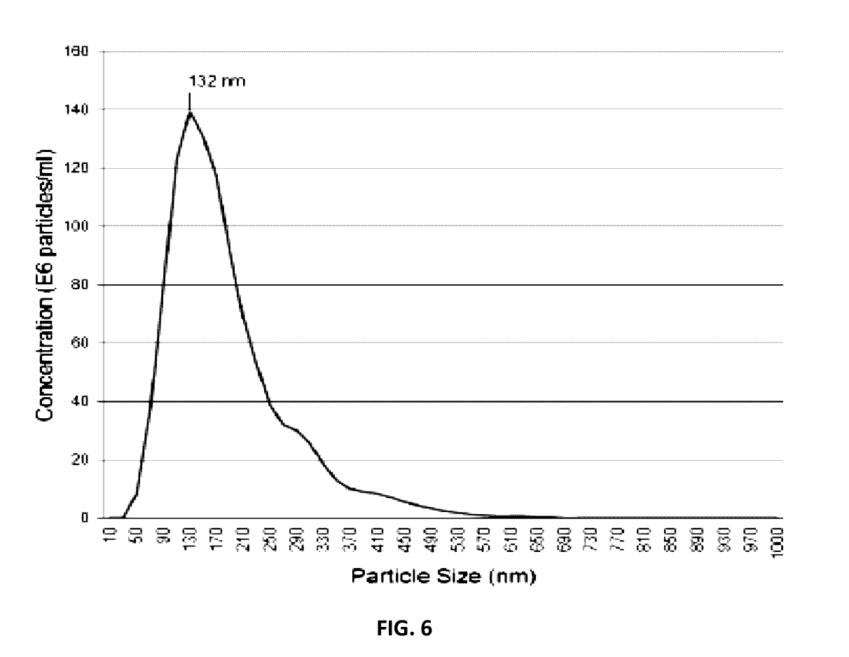

Enriched preparations of EVs were carried out (FIG. 6A). Verification of the isolation and enrichment of EVs compared to the cells of origin (K562 myelogenous leukemia and BJ primary fibroblast) was carried out using three methods: transmission electron (FIG. 1A) and immuno-electron micrographic techniques (FIG. 1B) and Western blot analyses of the EV specific membrane proteins compared to several cellular protein markers (FIG. 1C). The determination that the detected RNAs are cargos of the EVs rather than an artifact associated with EV purification was made treatment of preparation of EVs prior to RNA isolation with RNAse A and T1 and compared to RNA isolated from untreated EVs as well as EVs treated with detergent followed by RNAse (FIG. 1D). These results indicate the RNAs isolated from EVs were internalized within vesicles and thus protected from nuclease attack. Using a nanoparticle tracking technology (Nanosight Inc.) the number of EVs isolated from cultured 10⁸ K562 cells was conservatively estimated to be approximately 1.1×10¹¹ (FIG. 6B, Table 3). The number of EVs (quantified by Nanoparticle Tracking analysis) and quantity of RNA (quantified by Nanodrop) isolated from 1×10⁸ K562 and BJ cells is seen in Table 3.

TABLE 3

| Cells | Number of EVs | Quantity of RNA |
|---|---|---|
| K562 | 1.135 × 10¹¹ | 2-3 μg |
| BJ | 4.75 × 10⁹ | 800 ng-1 μg |

K562 cells were observed to have the most EVs released. A more typical EV production from the same number of cells was exemplified by the BJ cell lines of approximately 4.8×10⁹.

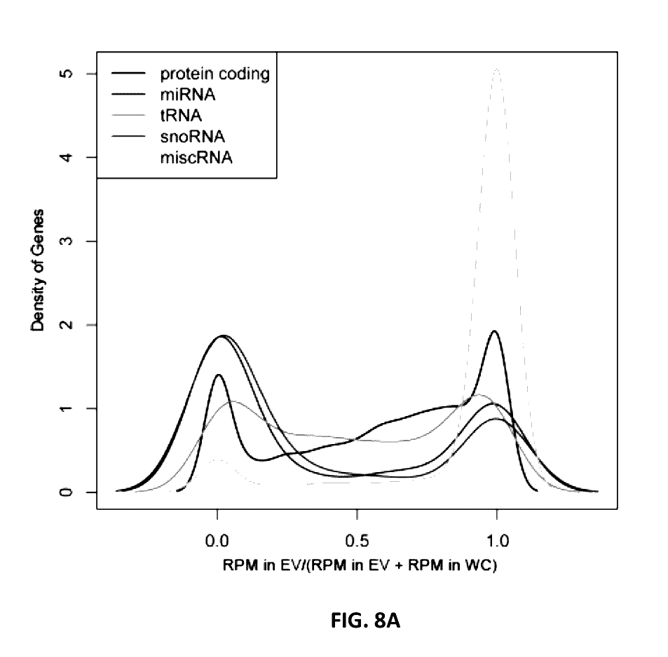

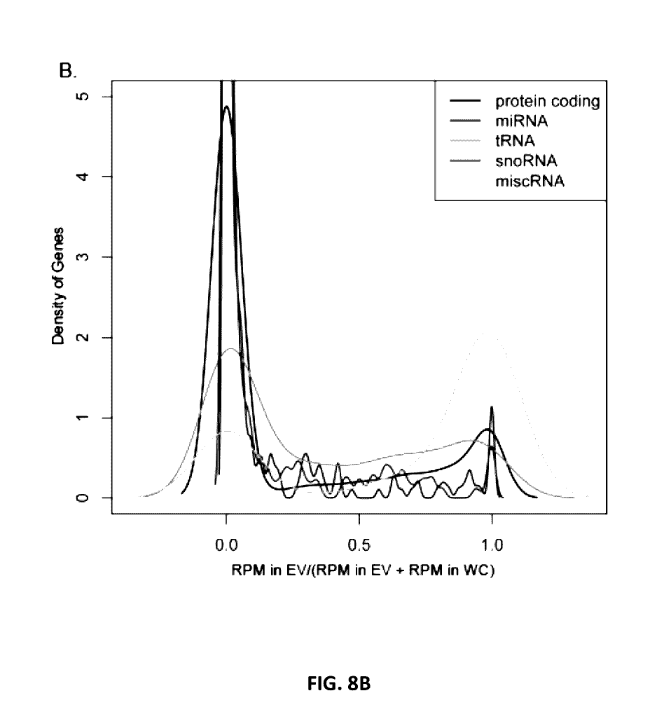

To study the RNA content of isolated EVs, an RNAseq profile analysis was performed on replicates of whole cells and EV cargoes derived from K562 (myelogenous leukemia) and BJ (foreskin fibroblast) cells. Profiles obtained from both cell lines and enriched EVs were highly reproducible (FIG. 7A, B). However, a low degree of correlation between RNA profiles in EVs and their source cells was readily evident. A detailed quantification of annotated sRNAs (reads per million [rpm]) isolated from BJ and K562 whole cells (FIG. 2A, B) indicated a predominance of rRNA, snoRNA, and miRNAs. In contrast, the relative distribution of sRNAs in EVs from the same cells indicates almost a considerable enrichment of the miscellaneous RNA (miscRNA) group and predominance of rRNA and tRNA (FIG. 2C, D). A comparison of the relative abundance of sRNA families between source cells and their EVs specifically highlights the enrichment of genes within the miscRNA group, consisting of several families of sRNAs—small Cajal body (sca), Y-RNA and vault (vt) RNAs (FIG. 8A, B). hY5 was the most abundant miscRNA gene present in EVs, composing 35% of all sRNAs in BJ EVs and 48% in K562 EVs. In contrast, hY5 accounts for only 0.1% and 0.2% of all reads from sRNAs within BJ and K562 whole cells, respectively. In EVs from both BJ and K562, the hY5 gene contributes over 89% of the reads from miscRNA, whereas in whole cells it constitutes only 40% of miscRNA reads, emphasizing the particular enrichment of this gene within EVs Enrichment levels of hY5 in EVs compared to whole cell RNAs from BJ and K562 were 196- and 68-fold, respectively.

### Example 26

### Processing of hY5 RNAs in EVs, but not in Whole Cells

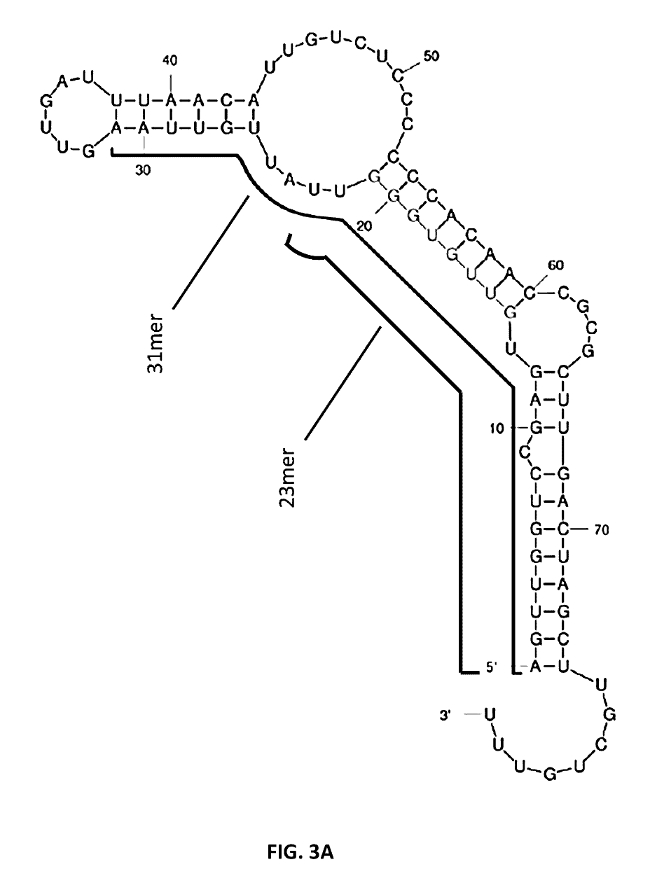

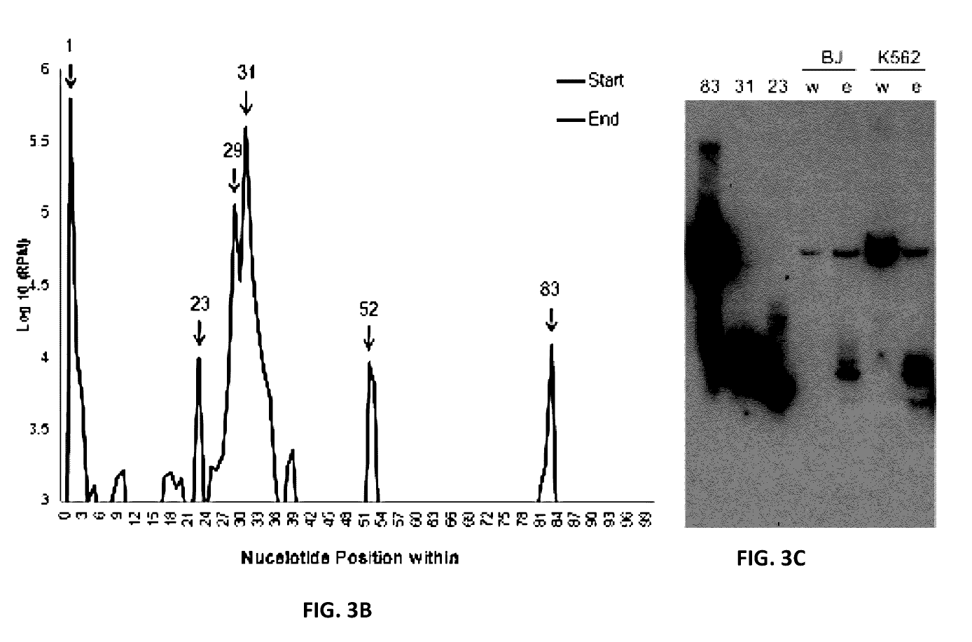

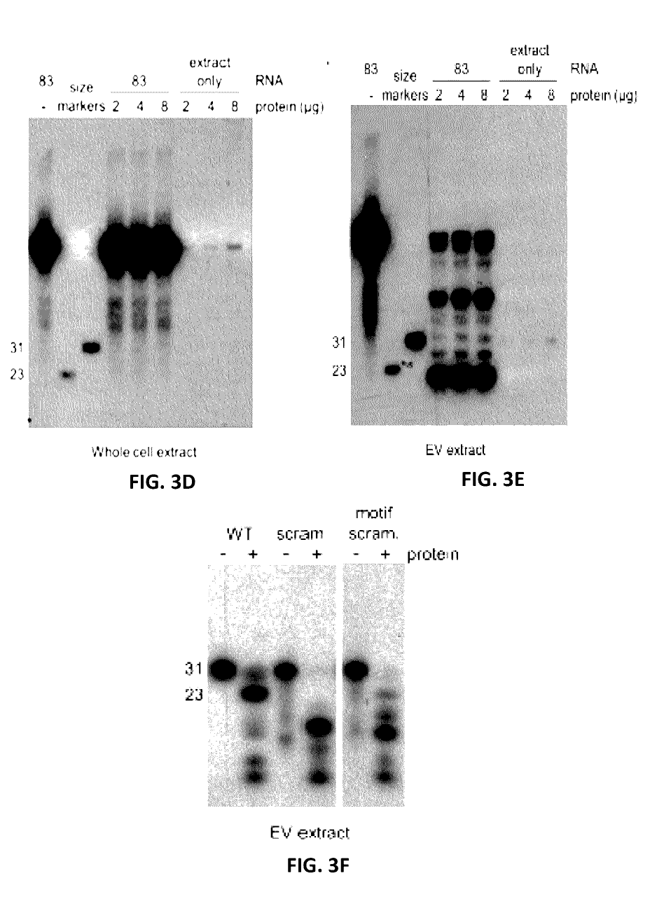

In the EVs, using RNAseq data, the 83nt hY5 primary transcript (FIG. 3A) was detected as well as shorter products of 23, 29, and 31nt in length, with start and end positions for each of these forms located at the 5′ end of the Gencode gene annotation (FIG. 3B). Additionally, a separate 31nt product mapping between nucleotide positions 51 to 83 of the primary transcript was observed, which was partially complementary to the 31nt 5′ fragment.

Northern hybridization analyses using a probe complementary to the first 31nt of the hY5 showed that the form of hY5 present in the whole cell was exclusively the full length 83nt transcript (FIG. 3C). While the RNA extracted from EVs contained the 83nt transcript, it was highly enriched for the 29-31nt forms, as well as a modest amount of a 23nt product, which was in agreement with the RNAseq results observed for the EV RNAs (FIG. 3B).

To further investigate the processing of hY5 seen in the EVs, a synthetic form of the 83nt hY5 transcript was incubated with K562 whole cell and EV protein extracts, followed with detection by Northern analysis. Synthetic copies of the 83nt hY5 incubated with K562 whole cell extracts exhibited no detectable processing (FIG. 3D), whereas incubation with K562 EV extracts led to dose dependent formation of all processed forms (23, 29, 31nt) detected in vivo (FIG. 3E). Additionally, a prominent hY5 processed species larger than 31nt was detected. The altered ratios of processed products and the appearance of a larger species in vitro, could be the result of the different conditions in an in vitro reaction (FIG. 3E). Treatment of the synthetic version of the 31nt RNA with K562 EV extract produced the same 23nt product as seen using the 83nt substrate (FIG. 3F) confirming that the 23nt product can be produced from either an 83nt or 31nt substrate. However, when a shuffled version of the 31nt RNA was treated with EV extract, no 23nt product was observed, demonstrating the sequence specificity of the processing activity of the EV extract (FIG. 3F).

A conserved double stranded sequence motif in the upper stem of all vertebrate Y-RNAs correlates with their participation in initiating DNA replication. Each of the products processed from the 5′ side of hY5 in vivo and in vitro contains a single stranded version of this motif. The motif was 8 nucleotides long (5′ GUU GUG GG 3′ (SEQ ID NO: 1)) extending from nucleotides 14-21 of hY5 (FIG. 3A). An alternate form of the 31nt substrate carrying a shuffled motif only exhibits residual processing into a 23nt product (FIG. 3F), underscoring the importance of the motif for processing of hY5 transcripts.

### Example 27

### Intercellular Transfer and Subcellular Localization of EVs and their RNA Cargoes

The transfer of EVs and their molecular cargoes from one cell type to another was demonstrated by use of both microscopic and molecular methods. The transfer of EVs between K562 and BJ cells and between K562 and two mouse cell lines (3T3 and HB4) was monitored. The goals of these experiments were to confirm the transfer of RNA content of EVs from one cell type to another in a species independent manner and to identify the subcellular localization and kinetics of the transferred EVs and RNA contents.

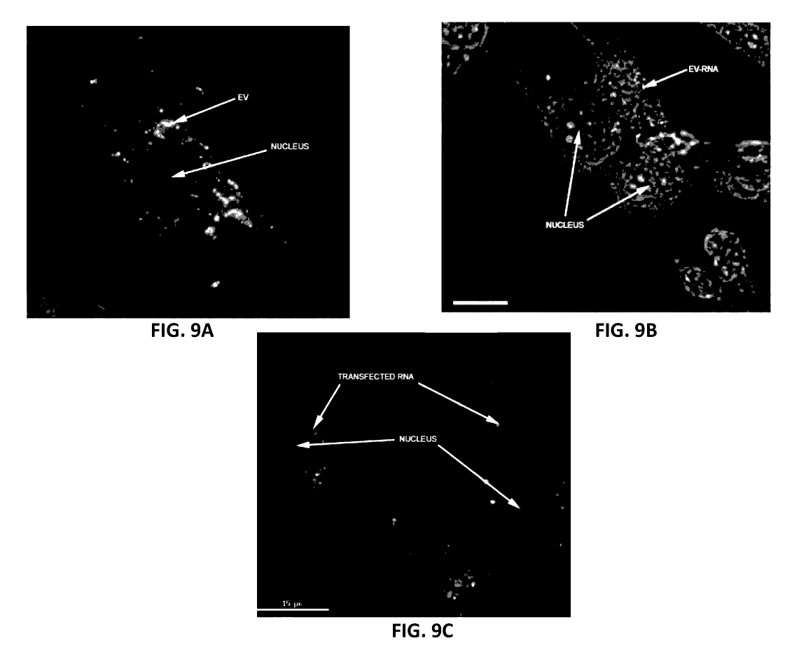

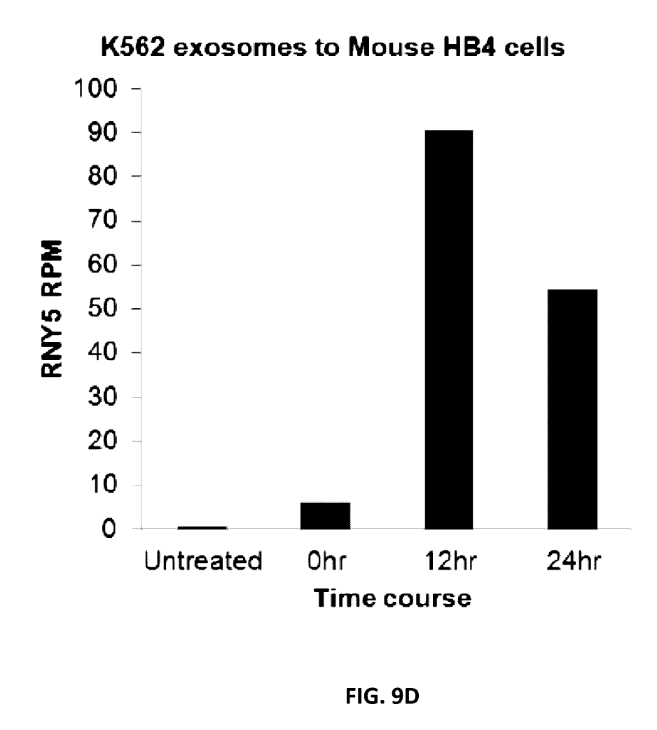

K562 EVs were first labeled with the lipid dye PKH67 after isolation. Following exposure of human BJ cells to labeled EVs, the EVs were found to be localized almost exclusively in the cytosol (FIG. 9A). To monitor the transfer of EV RNA, K562 cells were metabolically labeled with 5′ ethynyl uridine, and EVs were isolated. Transfer of labeled RNA contained in EVs was monitored after entry into mouse 3T3 cells. The localization of the labeled RNAs was also found to be primarily cytoplasmic (FIG. 9B). The same cytosolic localization was observed when primary human fibroblasts (BJ cells) were transfected with synthetic 31nt oligonucleotides versions of hY5 via lipofection (FIG. 9C). The larger and heterogeneous sizes of the lipofected vesicles distinguish these transfers compared to the EV transfers. These data also point to a lack of cell-type and species specificity in the transfer of the EVs. This former property was also observed with EVs from multiple human cell types transferred into different recipient cell lines.

The kinetics of intercellular transfer of EV RNAs was studied by treating mouse HB4 cells with EVs from human K562 cells followed by RNAseq analysis. Mouse cells were chosen for this experiment as a recipient cell type because of the absence of the hY5 gene in the mouse genome, allowing for the unambiguous monitoring of human hY5 transcripts. A temporal study lasting 24 hr revealed that maximum levels of hY5 were achieved by 12 hr post exposure followed by a progressive decrease in hY5 levels (FIG. 9D).

### Example 28

### Biological Phenotype of EVs and Processed hY5 RNAs

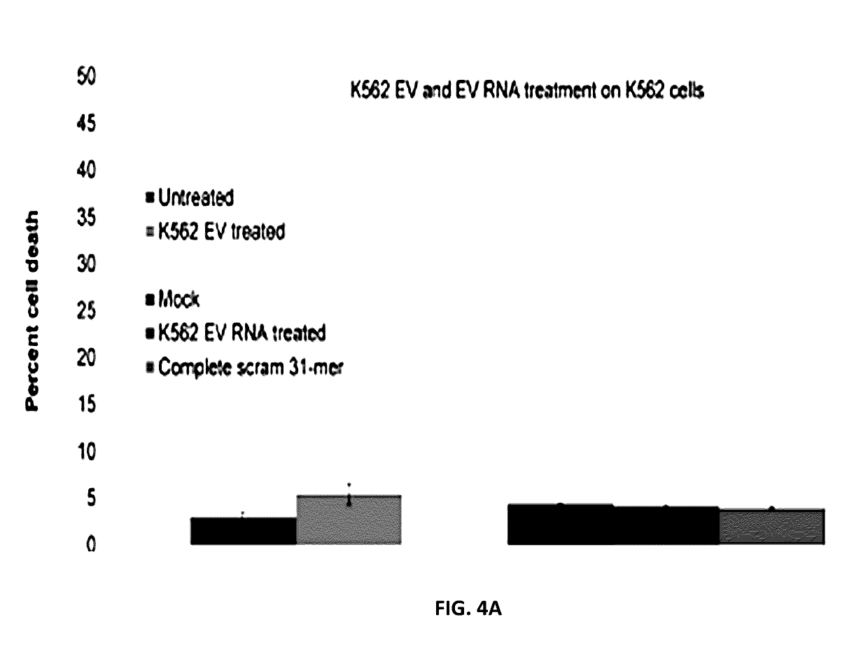

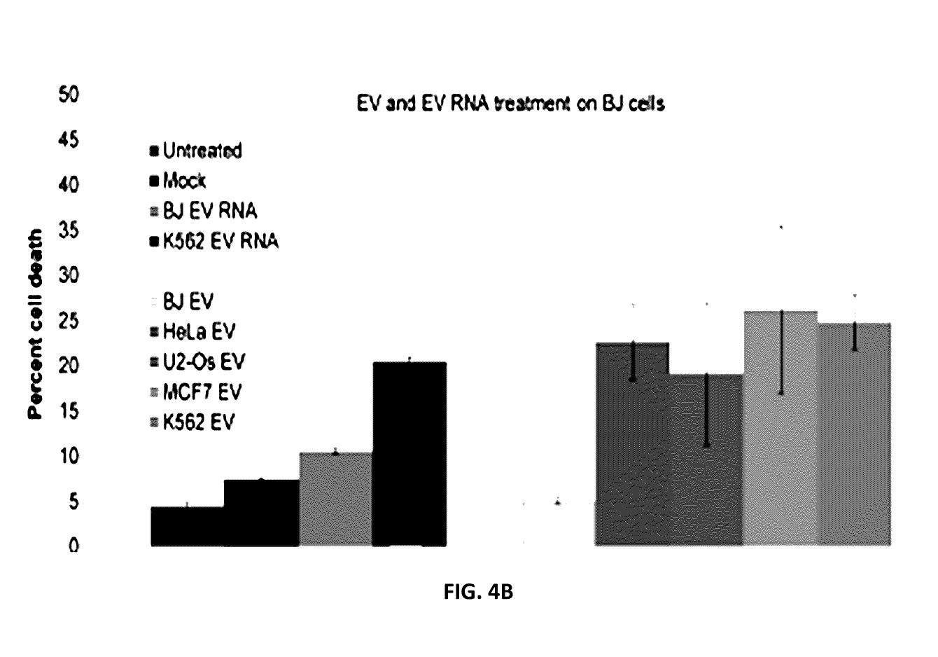

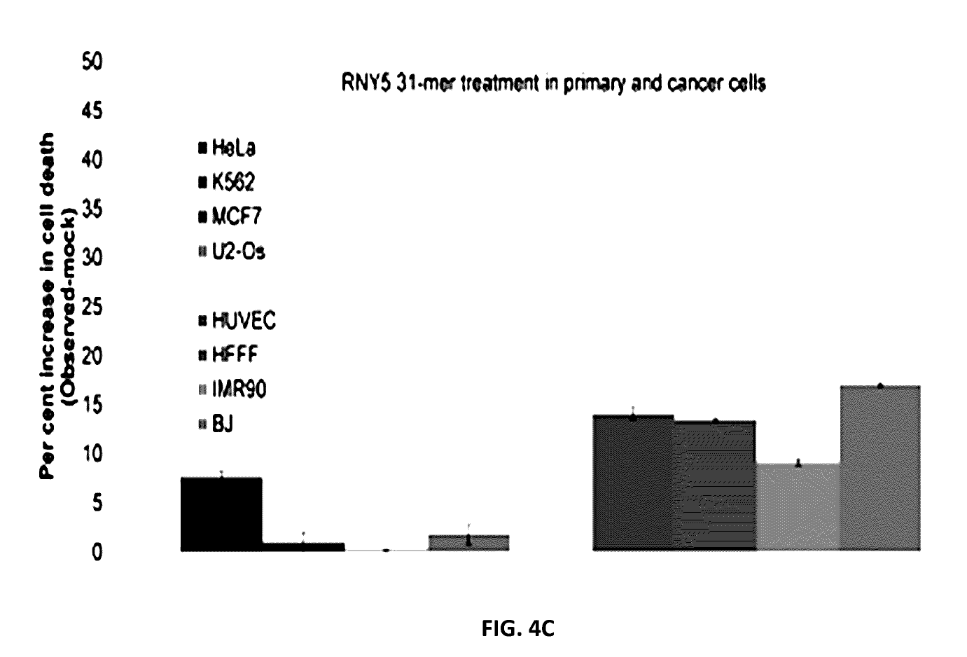

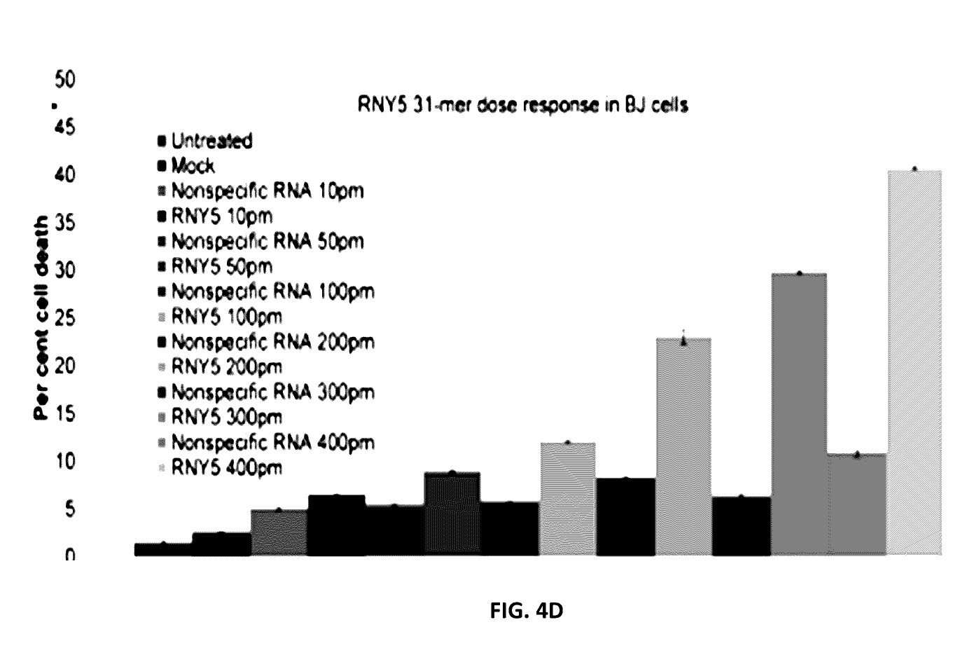

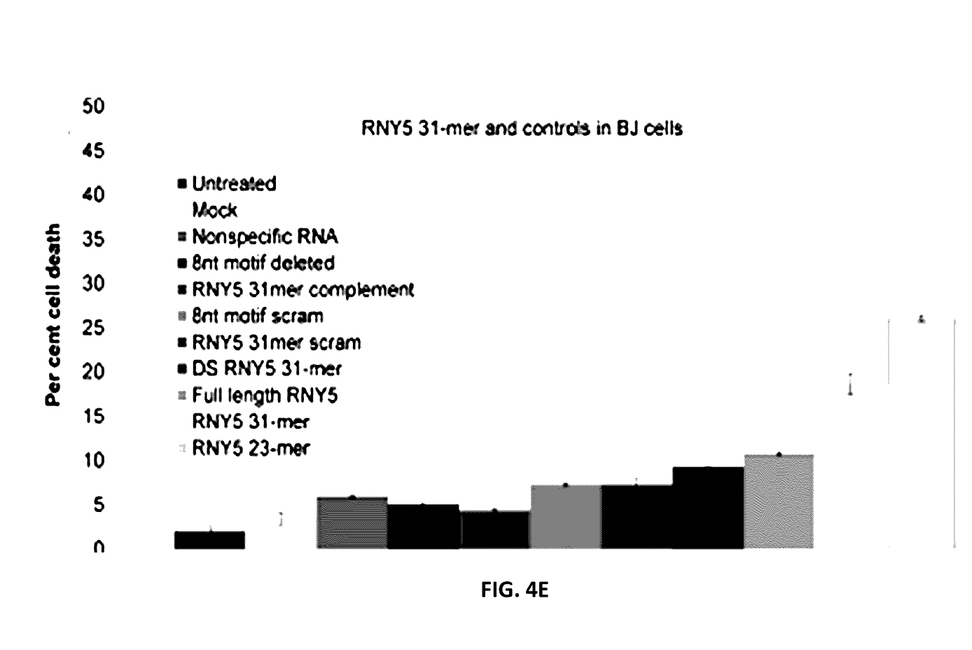

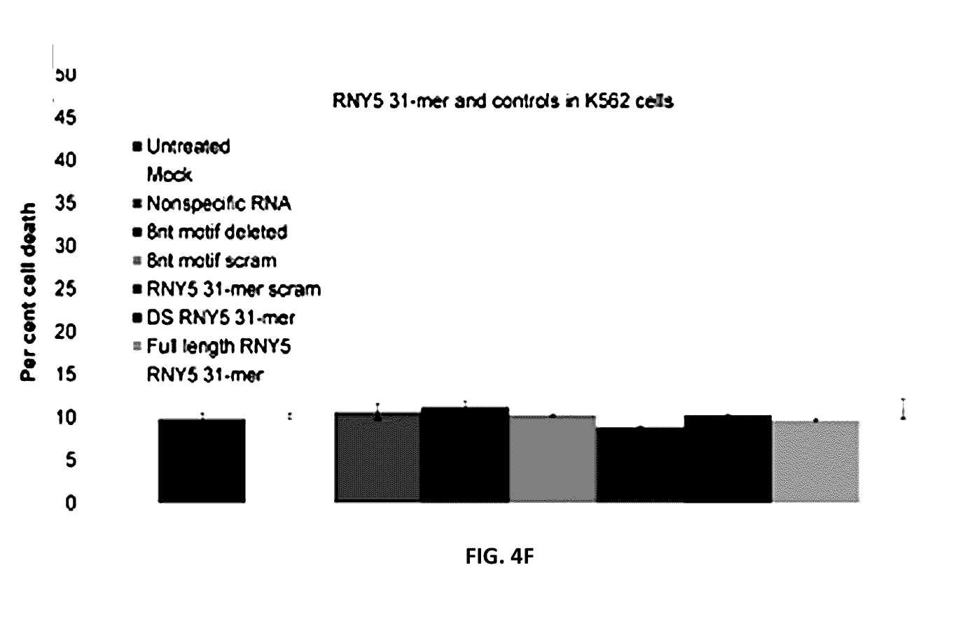

Using EVs isolated from the BJ human primary cells, and four cancer (K562, HeLa, U205, MCF7) cell lines, evaluations for the identification of phenotypic responses by cells taking up EVs were made. In each test, 2×10⁵ primary or cancer recipient cells were exposed to EVs from approximately 1×10⁸ cells. Exposure of BJ cells to BJ EVs or K562 cells to K562 EVs (FIG. 4A, B) resulted in no observable cellular phenotype. However, exposure of primary BJ cells to EVs from each of the cancer cell lines resulted in a relatively rapid cell death phenotype (FIG. 4B).

To determine if the causative agent triggering this cell death phenotype was the RNA cargo resident in the EVs, the totality of deproteinized and DNAse-treated RNA was isolated from each of the EV preparations obtained from the BJ and K562 cell lines. The total RNA preparations from each of the cell lines were then transfected via lipofection into the BJ and K562 cell lines. Transfection of total RNA obtained from K562 EVs resulted in an approximately two fold increase (10.6% vs. 20.5%) in the cell death of the BJ cells compared with BJ EV total RNA (FIG. 4B), while K562 cells were unaffected by the transfection of total K562 EV RNA (FIG. 4A).

Based on the significant abundance of the 31nt processed product from the 5′ side of hY5 in EVs, whether the cell death phenotype was specifically attributable to this RNA was investigated. A total of 4 human primary (BJ, IMR90, HUVEC, HFFF,) and 4 cancer (K562, HeLa, U205, MCF7) cell lines were each transfected with a synthetic version of 31nt processed hY5. Each of the primary cells tested exhibited a cell death phenotype while none of the cancer cell lines exhibited this phenotype (FIG. 4C). Varying the amounts of the synthetic 31nt RNA resulted in a dose-dependent cell death phenotype for BJ cells. (FIG. 4D).

Since other forms of hY5 can be detected in EVs, whether any of them may also contribute to the phenotype was investigated. Transfection of 23nt oligonucleotide in BJ cells induced comparable levels of cell death to that seen with the 5′ 31nt synthetic RNA (FIG. 4E). However, the 83nt full length hY5 RNA, the synthetic version of the 3′ 31nt fragment, and a double stranded version comprised of the 5′ and 3′ 31nt species induced substantially lower levels of cell death in BJ cells (FIG. 4E). The levels of cell death triggered by these synthetic RNA products and observed in K562 cells were all similar and at background levels (FIG. 4F). RNA sequencing statistics for all the different sequencing libraries from both K562 and BJ EVs and whole cells are shown in Table 4.

TABLE 4

| | K562 EV1 | K562 EV2 | K562 WC1 | BJ EV1 | BJ EV2 | BJ WC1 | BJ WC2 |
|---|---|---|---|---|---|---|---|
| Number of input reads | 15312204 | 38109015 | 37450624 | 12805596 | 13757050 | 13474063 | 18944518 |
| Average input read length | 28 | 38 | 60 | 30 | 43 | 62 | 55 |

UNIQUE READS:

| | | | | | | | |
|---|---|---|---|---|---|---|---|
| Uniquely mapped reads number | 5021255 | 16183627 | 28362474 | 3821688 | 5881994 | 10658696 | 13182629 |
| Uniquely mapped reads % | 32.79 | 42.47 | 75.73 | 29.84 | 42.76 | 79.11 | 69.59 |
| Average mapped length | 38.12 | 49.03 | 63.5 | 33.06 | 56.73 | 63.26 | 57.73 |

MULTI-MAPPING READS:

| | | | | | | | |
|---|---|---|---|---|---|---|---|
| Number of reads mapped to | 3778069 | 11996007 | 5116446 | 6452579 | 5784261 | 1620129 | 3399157 |
| multiple loci | | | | | | | |
| % of reads mapped to multiple | 24.67 | 31.48 | 13.66 | 50.39 | 42.05 | 12.02 | 17.94 |
| loci | | | | | | | |

UNMAPPED READS:

| | | | | | | | |
|---|---|---|---|---|---|---|---|
| % of reads unmapped | 42.54 | 26.05 | 10.61 | 19.76 | 15.2 | 8.87 | 12.47 |

To test whether the inability of double stranded versions of the RNA to cause the phenotype may be related to sequestration of the 8nt motif, the importance of which was demonstrated in the processing assays, its role in causing the phenotype was investigated. Cell death phenotype was lost when the motif was scrambled or deleted (FIG. 4E), further emphasizing the importance of this motif.

### Example 29

### Genome-Wide Gene Responses Associated with EV and Processed hY5

Comparison of transcriptional profiles prior to and 24 hr after treatment with EVs derived from K562 cells, as well as the synthetic version of the 31nt form of hY5 were made on two human primary cell lines (BJ and HUVEC). After 24 hr of treatment, a large number of annotated coding genes were seen in the EV treated cells to be differentially expressed by greater than two fold (BJ: 11,703 genes; HUVEC: 11,756 genes, of which almost half (5,574 genes) are common between the two cell types Similar number of genes seem to be differentially expressed using this threshold after treatment with the 31nt oligonucleotide (BJ: 9,311 genes; HUVEC; 12,061 genes), with a significant overlap between the two cell types observed here as well (3,748 genes). 1774 genes changed commonly between both types of treatment across both cell types, indicating that the 31nt hY5 fragment by itself was able to recapitulate a large part of the changes caused by EVs. Fold changes in genes within the TGF-β pathway after treatment of BJ and HUVEC cells with EV derived from K562 and the synthetic 5′ 31nt fragment are shown in Table 5.

TABLE 5

| | | BJ | | HUVEC | |
|---|---|---|---|---|---|
| Ensembl ID | Gene Name | EV | Y5 32-mer | EV | Y5 32-mer |
| ENSG00000011485 | PP5 | 2.976875179 | 3.421057404 | 0.0676865 | 0.0084047 |
| ENSG00000026103 | FAS | 4.656635422 | 2.048862289 | 0.009357006 | 0.563608352 |
| ENSG00000034152 | MKK3 | 0.09353015 | 0.0445025 | 0.0338432 | 0.0275619 |
| ENSG00000060656 | PTP | 3.061930644 | 0.326981718 | 0.0338432 | 0.02029715 |
| ENSG00000080839 | p107 | 0 | 0.0171034 | 0 | 1.876965838 |
| ENSG00000081189 | MEF2C | 0.117922946 | 1.08374729 | 0.583742961 | 0.421850359 |
| ENSG00000099942 | CRKL | 0.175407565 | 0.31541529 | 4.121847102 | 0.442392244 |
| ENSG00000100393 | p300 | 3.197199574 | 3.231212849 | 0.164448822 | 4.850764805 |
| ENSG00000100614 | PP2CA | 2.339504222 | 0.51617598 | 0.093728919 | 0.582309083 |
| ENSG00000105173 | CycE | 0.0531801 | 0 | 0 | 2.380874956 |
| ENSG00000105329 | TGFB | 2.058540504 | 1.693291308 | 0.131157533 | 0.403729161 |
| ENSG00000105810 | CDK6 | 4.544841922 | 2.544864042 | 0.025786698 | 12.85967948 |
| ENSG00000105851 | PI3K | 0 | 0 | 0.00216821 | 0.0202628 |
| ENSG00000106799 | TGFBR1 | 0.191907325 | 0.308684282 | 0.217953267 | 1.018430748 |
| ENSG00000108984 | MKK6 | 0 | 1.743880337 | 0 | 0.01867215 |
| ENSG00000110092 | CycD | 7.03248404 | 4.04805578 | 0.013477673 | 0.154756127 |
| ENSG00000110395 | CBL | 5.852682073 | 1.616081412 | 0.021747473 | 0.149827381 |
| ENSG00000111276 | KIP1 | 0.464566725 | 2.858382611 | 0.641712284 | 4.832878577 |
| ENSG00000112062 | p38 | 3.576198214 | 7.138180358 | 0.328897645 | 2.043336133 |
| ENSG00000116717 | GADD45 | 4.831559762 | 0.547287857 | 2.056499589 | 1.847321473 |
| ENSG00000117560 | FASL | 0 | 0 | 0 | 0 |
| ENSG00000120129 | MKP | 0.229483538 | 0.390175081 | 0.138351762 | 0.297863431 |
| ENSG00000123080 | INK4C | 0.613554654 | 1.582814268 | 0.152010301 | 2.406647683 |
| ENSG00000123374 | CDK2 | 6.12384958 | 6.836905194 | 0.082224316 | 1.471357954 |
| ENSG00000124762 | CIP1 | 4.745429033 | 3.980018471 | 0.132035636 | 3.616877976 |
| ENSG00000125952 | MAX | 0.434920714 | 0.406949119 | 1.025513794 | 0.692645795 |
| ENSG00000129355 | INK4D | 0.0198651 | 0.02980155 | 0 | 0.02029715 |
| ENSG00000129757 | KIP2 | 0.194830207 | 0.577973107 | 0.04777165 | 0.190159 |
| ENSG00000132646 | PCNA | 0.0198651 | 0.059603 | 0 | 0.02029715 |
| ENSG00000133740 | E2F5 | 0.497752197 | 0.433805695 | 0.015289917 | 0.22448795 |
| ENSG00000135446 | CDK4 | 3.166250701 | 0.666385609 | 0.019019883 | 0.308141895 |
| ENSG00000136997 | c-MYC | 10.50665047 | 5.430016103 | 0.059701521 | 2.688282508 |
| ENSG00000141510 | p53 | 1.98187379 | 3.096556868 | 0.0313505 | 0.269983647 |
| ENSG00000141646 | SMAD4 | 0.555839525 | 1.600218374 | 0.089552557 | 2.991030495 |
| ENSG00000142208 | AKT | 3.356141175 | 1.42663778 | 0.018674131 | 0.21442243 |
| ENSG00000145386 | CycA | 0.545009408 | 0.877365545 | 0.036757445 | 0.186117826 |
| ENSG00000147883 | INK4B | 0.167797249 | 0.182268818 | 0.046117279 | 1.966090745 |
| ENSG00000147889 | INK4A | 0.222979975 | 1.687551851 | 0.038680165 | 0.222069396 |
| ENSG00000150907 | FKHR | 4.826340311 | 0.435969251 | 0.095522544 | 0.411308598 |
| ENSG00000163513 | TGFBR2 | 8.577095271 | 3.259840411 | 4.335404091 | 5.448230223 |
| ENSG00000166949 | SMAD3 | 0.23328192 | 0.299832248 | 0.187844567 | 0.32353526 |
| ENSG00000167193 | CRK | 0.463050572 | 0.343327532 | 0.09405179 | 0.146491557 |
| ENSG00000168229 | DP1 | 0.0198651 | 0.0218838 | 0 | 0.02521415 |
| ENSG00000175197 | GADD153 | 1.612330178 | 1.296914308 | 0.014444114 | 0.231295431 |

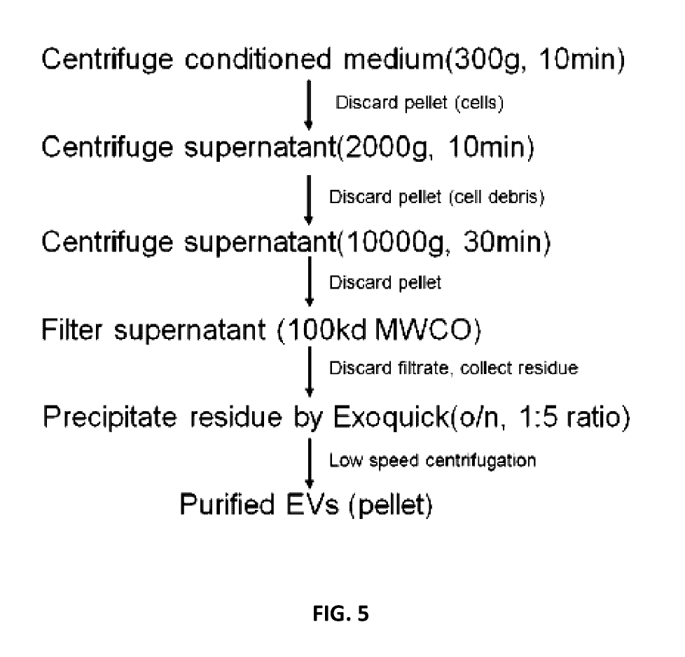

A gene set over-representation analysis for GO biological processes of the commonly differentially expressed genes indicated significant enrichment of genes from processes related to intercellular communication such as, regulation of cell signaling (p-value <6.4×10⁻⁵), regulation of cell communication (p-value <6.7×10⁻⁵), regulation of signal transduction (p-value <6.1×10⁻⁴), regulation of response to stimulus (p-value <9.3×10⁻⁴) and intracellular signal transduction (p-value <1.7×10⁻²) Similar gene set overrepresentation analysis on differentially expressed genes in HUVEC and BJ cell lines treated with the 31nt synthetic RNA and K562 EV taken separately, indicated that the genes involved in the regulation of cell death (p-value <4.64×10⁻³) and cell cycle (p-value <9.4×10⁻¹⁰) were significantly changed after EV treatment in both cell types, though these functional categories were not significant with only oligonucleotide treatment in both cells. The transcriptional profiles of primary cells treated with EVs from cancer cells was demonstrated to trigger differential expression of several genes associated with the FAS/TGF-β-Smad2/3 apoptotic pathway. These same genes were significantly altered both by treatment with EVs or oligonucleotides in both primary cell types tested (GO process—Signaling by TGF-beta Receptor Activating SMADs—EV treatment (p-value <4.4×10⁻⁸, hY5 treatment p-value <8.8×10⁻³). (FIG. 5). Also observable was the decrease in expression of the downstream Ink 4b which was a negative regulator of cyclin E, cyclin A and CDK2, and decreased expression of SMAD2/3/4 re-enforcing an apoptotic phenotype (FIG. 5). The absence of any potential cofactor accompanying the synthetic 31nt RNA, indicates that the RNA itself was sufficient to trigger the apoptotic phenotype (FIG. 4C).

### Example 30

### Evidence of Primary Cell Targeting by Cell to Cell Transfer

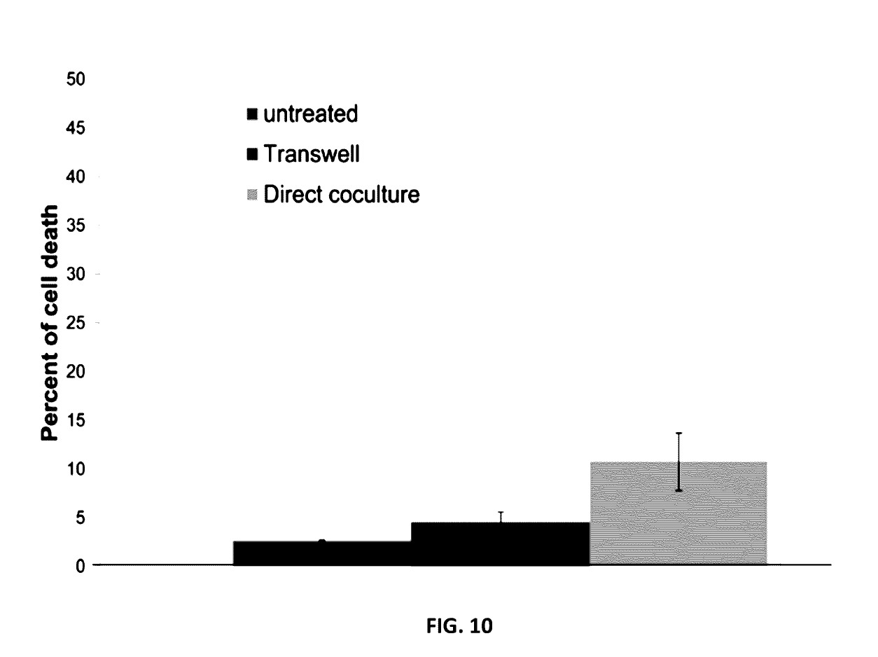

To determine if selective primary cell death caused by cancer cells present in numbers that favored neither cell type, co-culture of cancer and primary cells at 1:1 ratio (i.e., 2×10⁵ cells for each cell type) were carried out. Co-culture conditions were of two types, first involving cell to cell contact and second separate growth of each cell type in permeable trans-well culture conditions. Approximately four fold more cell death of primary cells (BJ) compared with untreated controls was observed in the cell to cell contact experiments (FIG. 10). The results using a trans-well assay approach in which the primary and cancer cell populations were separated by approximately 1 mm also demonstrated primary cell death, indicating that direct physical contact between cells and smaller volumes of media are not necessary for the occurrence of the phenotype.

### Example 31

### Materials

Total exosome isolation (from serum) reagent (Invitrogen), Total exosome isolation (from cell culture media) reagent (Invitrogen), Total exosome RNA and protein isolation kit (Invitrogen), blood serum from two donors, cell culture media from HeLa cells, 10×PBS, nuclease-free water (Ambion), 100% ethanol, nonoptical adhesive covers (Applied Biosystems), optical adhesive covers (Applied Biosystems), 384-well PCR standard plates (Applied Biosystems), 96-well PCR standard plates (Applied Biosystems), universal PCR master mix II (Applied Biosystems), human TaqMan miRNA assays, Veriti 96-well thermocyclers (Applied Biosystems), 7900HT Instrument, SW v2.3, TaqMan microRNA reverse transcription kit (Applied Biosystems), 1000 reactions, and Ion Total RNA-Seq kit v2 (Life Technologies) were utilized.

### Example 32

### Extraction of Exosomes from Cell Media Using Total Exosome Isolation Reagents

Fresh cell media was harvested from HeLa cells, grown in T175 flasks. Initially, the cells were grown in media containing 10% FBS (to ˜90% cell density), then washed twice with PBS and grown for the remaining 12 hr in 10% exosome-depleted FBS. The cell media samples were then centrifuged at 2,000 g for 30 min to remove cell debris. The supernatant containing the cell-free cell media was transferred to a fresh container and held on ice until use. Next, each sample was combined with ½ volume of total exosome isolation (from cell media) reagent and mixed well by vortexing or pipetting up and down until a homogenous solution was formed. Typical cell media volume utilized was 1 mL; however, the range of 100 μL-50 mL was used depending on the downstream application. The samples were incubated at 4° C. overnight and then centrifuged at 4° C. at 10,000 g for 1 hr. The supernatant was aspirated and discarded, and the exosome pellet was resuspended in PBS buffer and then stored at 4° C. short term (1-7 days) or −20° C. long term.

### Example 33

### Extraction of Exosomes from Human Blood Serum Using Total Exosome Isolation Reagents

Frozen serum samples were thawed in a water bath at room temperature until samples were completely liquid and then centrifuged at 2,000 g for 30 min to remove any cellular debris. The supernatant containing the cell-free serum was transferred to a fresh container and briefly held on ice until use. Next, each serum sample was combined with 0.2 volumes of Total exosome isolation (from serum) reagent and then mixed well by vortexing or pipetting up and down until a homogenous solution was formed. Typical serum volume utilized was 100 μL; however, the range of 50 μL-5 mL was used depending on the downstream application. The samples were incubated at 4° C. for 30 min and then centrifuged at room temperature at 10,000 g for 10 min. The supernatant was aspirated and discarded, and the exosome pellet was resuspended in PBS buffer and then stored at 4° C. short term (1-7 days) or −20° C. for long term.

### Example 34

### Sizing and Quantification of Exosomes with Nanosight LM10 Instrument

Exosomes purified from cell media and blood serum were diluted with PBS buffer (10-5000× in order to have the nanovesicle concentration in the working range for the Nanosight LM10, 2×10⁸-8×10⁸) and then quantified and sized using the Nanosight LM10 instrument (Nanosight, UK), following the manufacturer's protocol. The LM10 uses a laser light source to illuminate nanoscale particles (10-1000 nm) which are seen as individual pointscatters moving under Brownian motion. The paths of the point scatters, or particles, are calculated over time to determine their velocity which can be used to calculate their size independent of density. The image analysisntA software compiles this information and allows the user to automatically track the size distribution and number of the nanoparticles.

### Example 35

### Western Blot Analysis

Exosome samples isolated from cell media or blood serum (typically equivalent of 50 μL cell media and 5 μL serum) were mixed with 2× nonreducing Tris-glycine SDS sample buffer (Novex) for CD63, and 2× reducing buffer for CD9, then heated at 75° C. for 5 min and loaded onto a 1.5 mm×15 well 4-20% Tris-Glycine gel (Novex). Benchmark prestained protein ladder (Invitrogen) was added to one well as a control to monitor the molecular weight of the protein samples. The gel was run under denaturing conditions at 150 V for 1.5 hr and then transferred to a membrane using the iBlot instrument (Life Technologies). After transfer, the membranes were processed on the BenchPro 4100 (Life Technologies) with CD63 or CD9 antibody diluted 100 μg into 20 mL. The WesternBreeze Chemiluminescence kit was utilized on the next step; membranes were exposed to X-ray film for 1-10 min and the film was analyzed.

### Example 36

### RNA Recovery Using the Total Exosome RNA and Protein Isolation Kit

The Total exosome RNA and protein isolation kit (Invitrogen) was utilized for recovery of RNA from the exosome samples obtained with the reagent and ultracentrifugation protocol and parental samples for each sample type, HeLa cell pellets (1×10⁶ cells) and cell-free serum. 200 μL of each sample (brought up to volume with PBS if necessary) was combined with 205 μL of 2× denaturing solution, vortexed to lyse, and then incubated on ice for 5 min. After incubation, 410 μL of acid-phenol:chloroform was added to the mixture and vortexed for 30-60 s to mix. Samples were then centrifuged for 5 min at 10,000 g at room temperature to separate the mixture into aqueous and organic phases. Once centrifugation was complete, the aqueous (upper) phase was carefully removed without disturbing the lower phase or the interphase and transferred to a fresh tube. 1.25 volumes of 100% EtOH was added to the aqueous phase for each sample and then vortexed to mix. 700 μL of volume was placed onto spin column in a collection tube and then spun at 10,000 g for 15 s to move the sample through the filter cartridge. Samples were then washed once with 700 μL wash solution 1 and 2× with 500 μL wash solution ⅔ (centrifuged at 10,000 g for 15 sec for each wash). After washing, filter was dried by spinning for an additional 1 min at 10,000 g. The filter cartridge was transferred into a fresh collection tube and 50 μL of preheated (95° C.) nuclease-free water was applied to the center of the filter. Samples were centrifuged for 30 sec at 10,000 g to recover the RNA, and then a second 50 μL volume of preheated (95° C.) nuclease-free water was applied to the center of the filter and centrifuged for 30 sec at 10,000 g. After the second spin, the eluate containing the RNA was collected and stored at −20° C. For cell pellet RNA, a DNase treatment was performed using the DNase-free Kit (Ambion) to remove any contaminating DNA; DNase treatment was not performed on exosome samples as they had a much smaller input. After treatment, each sample was diluted to 2 ng/μL and 1 μL was analyzed on the Agilent 2100 Bioanalyzer using the Agilent RNA 6000 Pico Kit (Series II) to determine the mass of RNA going into downstream analysis.

### Example 37

### Reverse Transcription and Quantitative Real-Time PCR (qRT-PCR) Analysis of the RNA Sequences Isolated from the Exosomes

Reverse Transcription (RT) Master Mix was prepared for each sample using the TaqMan MicroRNA Reverse Transcription Kit reagents and protocol (Applied Biosystems) with hY5 specific RT primers). 10 μL of the RT master mix was added to corresponding wells in a 96-well plate, and 5 μL of each sample was added to the master mix. Plates were covered with adhesive (nonoptical) cover and spun down to remove air bubbles and then placed into a 9700 thermocycler and incubated as follows: 4° C. for 5 min, 16° C. for 30 min, 42° C. for 30 min, and 85° C. for 5 min. Reactions were kept at 4° C. until use.

qPCR master mixes were prepared for each of five microRNAs by combining 5 μL of AB Universal PCR Master Mix II, 2.5 μL of nuclease-free water, and 0.5 μL of the 20× TaqMan assay. After mixing, 8 μL of each master mix was placed into wells in a 384-well plate (enough for triplicate reactions for each isolation replicate). Two μL of each RT reaction was added in triplicate to the master mix of each target and the plates were sealed with an optical adhesive cover. Plates were spun down to remove air bubbles and then placed into a 7900HT instrument and run using the following thermocycler protocol 95° C. for 10 min+(95° C. for 15 s; 60° C. for 60 s) for 40 cycles. Once the run was complete, automatic Ct analysis was performed with SDS v2.3 software, and average and standard deviations were calculated for each set of isolations and qPCR reactions for each target.

### Example 38

### Preparation of the Small RNA Libraries and Sequencing Exosomal RNA

Small RNA libraries were prepared using the Ion Total RNA-Seq Kit v2 (Life Technologies) protocol and materials. However, a number of modifications were introduced into the RNA-Seq protocol in order to accommodate the specific nature of the exosome samples: (1) relatively low amount of RNA and (2) majority of the RNA cargo being <200nt in size. For library construction, the RNA sample was dried down to 3 μL and then combined with the hybridization reagents and incubated at 65° C. for 10 min and 16° C. for 5 min Ligation reagents were then added and the samples were incubated for 16 hr (overnight). After ligation, reverse transcription was performed: RT master mix was added to the samples, tubes were incubated at 70° C. for 10 min, samples were snap-cooled on ice, the RT enzyme was added, and the samples were incubated at 42° C. for 30 min. cDNA from the RT reaction was purified using the kit's clean-up module containing MagMAX Beads (5 μL per well of a 96 well plate) and eluted in 12 μL of nuclease-free water. Six μL of the purified cDNA was combined with PCR primers and Platinum PCR SuperMix High Fidelity reaction mix was then placed in a thermocycler and amplified using the following protocol: 94° C. for 2 min (94° C. for 30 s, 50° C. for 30 s, and 68° C. for 30 s) 2 cycles; (94° C. for 30 s, 62° C. for 30 s, and 68° C. for 30 s) 16 cycles; 68° C. for 5 min. Once protocol was complete, reactions were stored on ice until purification. The amplified DNA (final library) for each sample was purified using the kit's clean-up module containing MagMAX Beads (5 μL per well of a 96-well plate) and eluted in 10 μL of nuclease-free water. Final libraries were stored on ice for the short term and at −20° C. for long term. To assess the yield and size distribution, 1 μL of the library was run on an Agilent DNA High Sensitivity chip (Agilent). The molar concentration of the library was determined with the Agilent 2100 Bioanalyzer Instrument Expert software and used to dilute libraries to correct concentration for sequencing. Sequencing was performed for each sample on the Ion Torrent PGM instrument using 318 chips (11,000,000 wells per chip) and the protocol listed in the Total exosome RNA and protein isolation kit (Invitrogen) with 160 flows (40 cycles).

### Example 39

### Nuclease and Protease Protection Assays

For RNAseA protection assays, exosomes were inubated with 4U/mL RNAseA (Sigma) while or PBS buffer. After 30 min incubation at 37° C., an adequate volume of Trizol LS was added to denature the RNAse and proceed to RNA isolation as previously indicated. For protease protection assays, EVs were incubated with proteinase K (Sigma) at 64 g/mL. After 30 min incubation at 37° C., phenylmethylsulfonyl fluoride (PMSF; Sigma) was added at 5 mM final concentration. After protease inhibition, 4U/mL RNAseA or buffer was added. Samples were incubated for 30 min at 37° C. and subjected to RNA isolation. RNA from both assays was analyzed by SL-RTqPCR specific to hY5. The Cq values for paired samples were calculated and taken together to determine the effect of RNAse treatment versus no treatment, and of protease followed by RNAse versus protease alone.

### Example 40

### Novel Exosome Isolation Method

Comparative analysis of methods based on multiple parameters such as yield, efficiency and morphology of exosomes and consistency in detection of exosomal cargo has reported unique advantages and limitations of each method. While the initial differential centrifugation steps in ultracentrifugation method ensures the depletion of majority of other particles present in the medium or body fluid such as microvesicles and apoptotic bodies, the efficiency of exosomes isolation using ultracentrifugation was low and the effect of high gravitational force on the integrity and morphology of exosomes and its cargo was poorly understood. Precipitation based approach are prohibitively expensive, for example when preparing RNA-Seq libraries. Centrifugal ultrafiltration often runs the risk of loss of vesicles due to clogging or membrane fouling.

The limitations of these individual approaches has led to the development of a novel ‘hybrid’ approach which combines the unique advantages of each of these individual methods into one unifying hybrid approach. The first three low speed differential centrifugation steps of ultracentrifugation method deplete larger non-exosomal contaminants, which can be performed relatively quickly without an expensive ultracentrifuge. Ultrafiltration with 100 kDa NMWL membrane reduces large volumes of media to only a few ml of exosomes enriched residue, which makes liquid handling quick and easy. Most importantly, this step allows quick and affordable downstream use of Exoquick based “gentle” precipitation of exosomes from large volumes of media otherwise not possible. Thus, by combining low-speed differential centrifugation steps of ultracentrifugation with 100 kDa membrane centrifugal ultrafiltration and followed by Exoquick based precipitation of exosomes, a novel hybrid approach of exosome isolation which was easy, highly efficient, consistent and scalable was developed. A comparative study of this approach with conventional ultracentrifugation, precipitation and ultrafiltration based approaches based on multiple parameters, including yield and size distribution of isolated exosomes and exosomal RNA, scalability as well as reproducibility in detection of RNA to demonstrate the superiority of the hybrid approach was exemplified below.

### Cell Culture and Isolation of Exosomes

Exosomes were isolated in replicates by four different methods, namely Ultracentrifugation, ultrafiltration, Precipitation using Exoquick-TC and Hybrid method.

Ultracentrifugation:

Briefly, 200 ml of conditioned medium was centrifuged at 300 g for 10 min to discard the cell pellet. The supernatant was centrifuged at 2000 g for 10 min and the pellet comprising of cell debris and apoptotic bodies was discarded. The supernatant was centrifuged again at 10,000 g for 30 min and the microvesicles pellet was discarded. The supernatant was ultra-centrifuged at 110,000 g for 70 min using Sorvall SW-28 rotor. The supernatant was discarded and the pellet composed of exosomes and protein complexes were suspended in PBS. The exosomes was centrifuged again at 110,000 g for 70 min. The supernatant was discarded again and the pellet was suspended in 500 microliter PBS.

Precipitation:

exosome isolation was performed by Exoquick-TC from 50 mL of conditioned medium (1×10⁷ source cells approx.) due to prohibitive expense of the precipitation reagent Exoquick-TC (System Biosciences). Briefly, conditioned medium was centrifuged at 300 g for 10 min The cell pellet was discarded and the supernatant was centrifuged at 2000 g for 10 min The pellet, comprising of cell debris and apoptotic bodies was discarded. 10 mL of Exoquick-TC was added to the 50 mL supernatant (1:5 ratios) and incubated for 12 hr at 4° C. Next day, the conditioned media-Exoquick-TC mixture was centrifuged at 1500 g for 30 min. The supernatant was discarded and the pellet was centrifuged again at 1500 g for 5 min. The left over supernatant was discarded and the pellet was suspended in 500 μL PBS. Yield of exosomes and exosomal RNA from 200 mL conditioned medium using precipitation was extrapolated by multiplying the yield by a factor of 4.

Ultrafiltration:

200 mL of conditioned medium was centrifuged at 300 g and 2000 g to discard the cells and cell debris pellet respectively. Microvesicles and other larger vesicles were first depleted using from the supernatant by ultrafiltration using 0.45 μm polycarbonate filter (Sterivex, Millipore). The filtrate was then further filtered using Centricon Plus-70 100 KD filters (about 10 nm pore size). The isolated exosomes in the residue was collected and the filtrate was discarded. The volume of the collected exosomes was brought to 500 μL with PBS.

Hybrid:

200 mL of conditioned medium was centrifuged at 300 g for 10 min. The cell pellet was discarded and the supernatant was centrifuged at 2000 g for 10 min. The pellet, comprising of cell debris and apoptotic bodies was discarded and the supernatant was centrifuged at 10000 g for 30 min. The microvesicles pellet was discarded and the supernatant was filtered with Centricon Plus-70 100 KD (10 nm pore size approx.) centrifugal filters at 3500 g for 15 min. The residue, enriched in exosomes was collected while the filtrate was discarded. The volume of the filtration residue was made to 500 μL using PBS.

Nanoparticle Tracking Analysis (NTA):

Nanoparticle tracking analysis was performed on the purified exosomal samples using Nanosight LM10. The samples were run at 25° C. using PBS as a diluent.

Transmission Electron Microscopy (TEM):

Aliquots of exosomes suspensions were dispensed on parafilm on a petri dish and Butvar coated EM grids were adsorbed on them for 5 min at room temperature and then kept on ice. The grids were transferred to drops of distilled water thrice for 30 s each to wash off excessive salts. The grids were then transferred to a drop of 1% uranyl acetate in 1% methyl cellulose for 30 s followed by another transfer to a second drop for 5 min. The grids were air dried and excess stain was blotted off. Imaging was performed using Hitachi H7000 electron microscope at 75 kV.

Isolation of RNA:

Purified exosomes, re-suspended in PBS, were treated with 15 μL of RNAse cocktail (Ambion) at 37° C. for 30 min to degrade any free RNA molecules that was not enclosed within exosomes. The RNAses were immediately inactivated with the lysis/binding buffer of mirvana miRNA isolation kit (Ambion) and immediately proceeded to total RNA isolation using manufacturer's protocol and ethanol precipitated with 2.5 volumes of 100% ethanol and 0.25 volumes 3M sodium acetate. The precipitated RNA was treated with Turbo-DNase (Ambion) and precipitated with ethanol. The re-suspended RNA was quantified using an Agilent Bio-analyzer RNA pico-chip.

Small RNA-Sequencing:

Small RNA libraries were constructed using Illumina TruSeq Small RNA Sequencing kit. The purified RNA samples were first treated with Tobacco Acid Pyrophosphatase (TAP) for 1 hr at 37° C. to convert 5′ capped and triphosphate RNA molecules into monophosphate and make then amenable to adapter ligation. The RNA was subsequently extracted using phenol-chloroform and precipitated with 2.5 volumes 100% ethanol and 0.25 volumes sodium acetate. The precipitated RNA sample was then used for adapter ligation, reverse transcription and PCR amplification. While Ultracentrifugation, hybrid and filtration libraries were amplified for 15 PCR cycles, libraries from Precipitation method were amplified for 30 PCR cycles due to its extremely low starting input of RNA. The amplified cDNA was run on a 2% agarose gel and region pertaining to 20-200 bp of RNA (145-350 bp cDNA on gel) were cut out of the gel. The cDNA was then extracted using Qiagen gel extraction kit according to manufacturer's protocol, ethanol precipitated and quantified using Bioanalyzer HS-DNA chip. Finally, replicates of libraries were multiplexed and run on Illumina HiSeq 2000 or MiSeq.

### Comparison by Yield and Size Distribution of Exosomes:

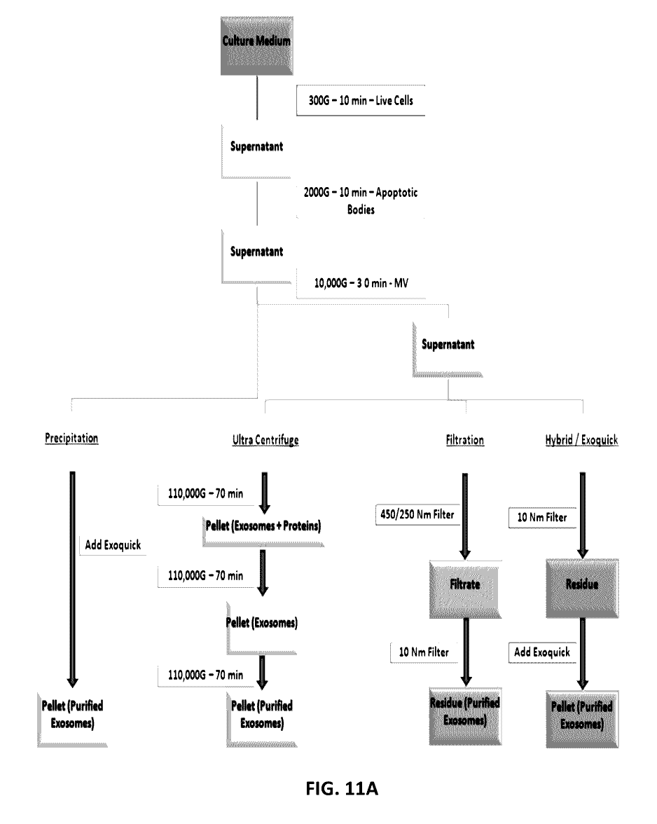

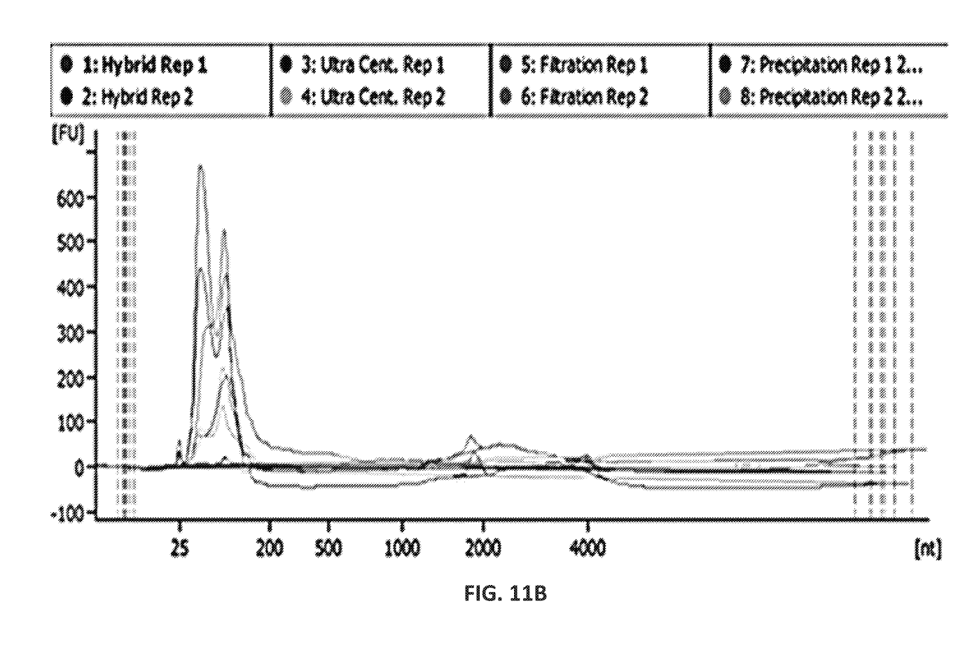

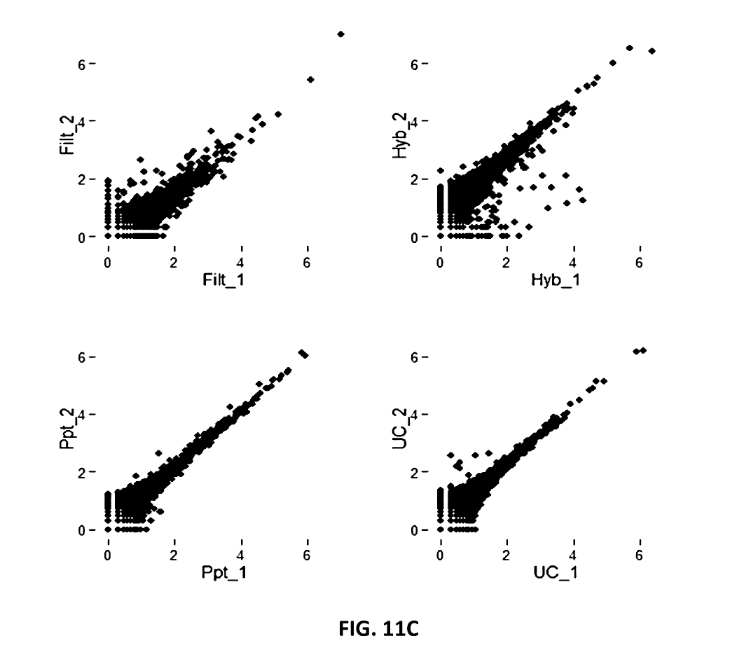

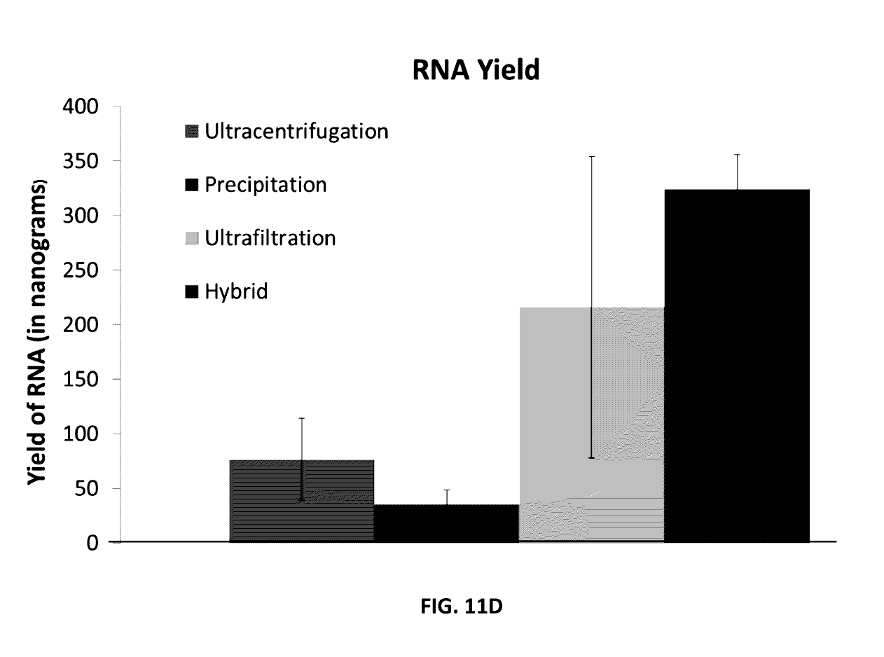

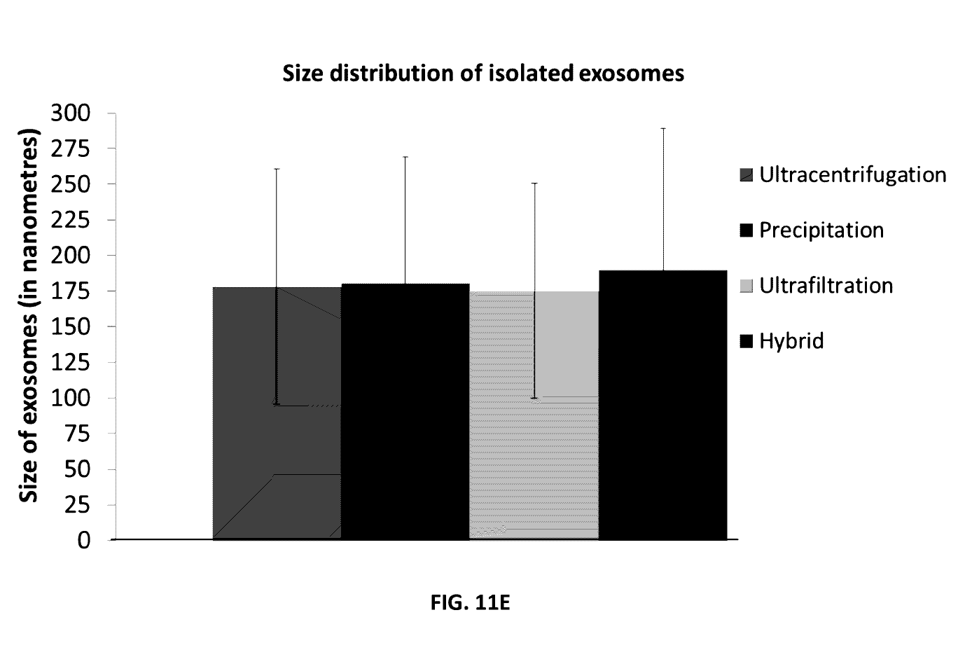

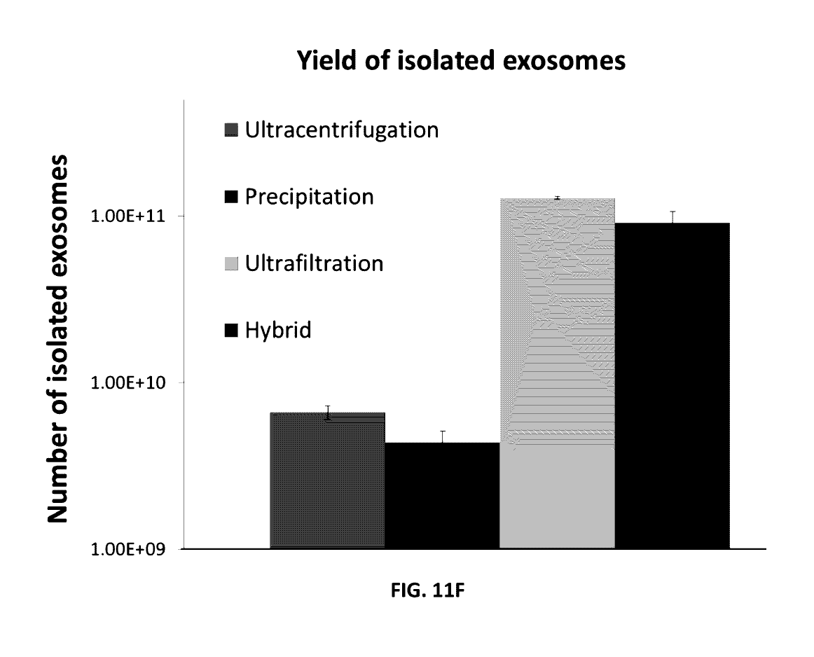

The yield of purified exosomes achieved was an important parameter to assess the isolation methods. Nanoparticle tracking analysis allowed us to quantify and compare the number of exosomes isolated by each isolation method. The hybrid method yielded 1.06×10″ and 7.59×10¹⁰ exosomes. In contrast, conventional ultracentrifugation methods isolated 7.27×10⁹ and 6×10⁹ exosomes. Replicates of ultrafiltration yielded 1.31×10¹¹ and 1.26×10¹¹ exosomes respectively and precipitation method yielded 5.10×10⁹ and 3.68×10⁹ exosomes. Thus, although the yield of exosomes by the hybrid method was slightly lower than ultrafiltration method, the yield from hybrid method was higher than the traditional ultracentrifugation and filtration methods by at least an order of magnitude (FIG. 11A).

NTA analysis also allowed us to compare the size distribution of the isolated exosomes. The hybrid method isolated vesicles of remarkably similar size distribution when compared with other methods. The mean diameter of exosomes isolated in replicates by hybrid method was 185 nm and 195 nm, with standard deviation of 89 nm and 109 nm respectively, while ultrafiltration method isolated exosomes of mean 173 nm and 177 nm with standard deviation 80 nm and 71 nm respectively. Similarly, while precipitation method isolated exosomes of mean diameter 173 nm and 188 nm with standard deviation 84 nm and 93 nm respectively, the ultracentrifugation method isolated exosomes of mean diameter 183 nm and 173 nm with standard deviation 84 nm and 81 nm respectively (FIG. 11E). Thus, the size distribution profile of exosomes isolated by the hybrid method are remarkable consistent with all other methods.

### Comparison by Yield and Size Distribution of RNA

The quantity and size distribution of the RNA molecules enclosed in exosomes was determined. Bioanalyzer profiles showed that each of the methods resulted in isolated exosomes consisting of mostly small RNAs of less than 200nt. The amount of long RNA (>200nt) present in exosomes was very low. The RNA size distribution profile obtained by the hybrid method was found to be remarkably consistent and displayed much overlap with the RNA size distribution obtained with established methods (FIG. 11E).

The yield of RNA isolated by the four methods also varied greatly within the replicates of each method (FIG. 11D). Replicates of the Hybrid method isolated 356 ng and 292 ng of RNA. In contrast, replicates of Ultracentrifugation method yielded 114 ng and 38.8 ng of RNA. The Precipitation method yielded 48.8 ng and 22 ng of RNA. Surprisingly, RNA yield from ultrafiltration showed inconsistency among replicates. While, a first replicate of ultrafiltration method yielded 354 ng of RNA, a second replicate yielded just 77.8 ng of RNA. The highest and most consistent yield of RNA from exosomes was achieved by the Hybrid method. This strongly underscores the ability of hybrid method to purify structurally intact exosomes resulting in minimal RNA loss.

### Comparison by RNA-Seq

RNAseq analysis allowed assessment of the degree of reproducibility in detection achieved by each of the isolation methods as well as the consistency of detection among the four methods. Illumina TruSeq small RNA-Sequencing was performed on the exosomal RNA isolated by the four methods (in duplicates). Each library was sequenced (Table 6) and mapped using STAR. The proportion of reads mapping to the genome was determined and was highly consistent among the libraries. The percentage of reads that mapped uniquely to the genome was determined and the percentage of reads that mapped to multiple locations in the genome was also determined (Table 6). The average read length around was determined and the read length distribution of the libraries was extremely similar to each other.

TABLE 6

| | Hyb | Ppt | UC |
|---|---|---|---|
| Filt | 0.840 | 0.849 | 0.879 |
| Hyb | | 0.878 | 0.927 |
| Ppt | | | 0.880 |

To investigate the inherent consistency of detection of exosomal RNA obtained by each method, the Pearson's correlation coefficient between the replicates of each method was utilized. While each method demonstrated strong correlation between its replicates, the highest correlation was observed using the precipitation (r²=0.91) method. Ultracentrifugation and hybrid method came a close second and third, with correlation coefficients of 0.89 and 0.87, respectively. Replicates of ultrafiltration had a relatively weak correlation with a coefficient (r²) around 0.8 (Table 7).

TABLE 7

| Method | Correlation |
|---|---|
| Filtration (450 Nm) | 0.8002574 |
| Hybrid | 0.8735242 |
| Precipitation | 0.9158689 |
| Ultra Centrifugation | 0.8996195 |

Since ultracentrifugation has traditionally been recognized as the “gold standard” method for isolation of exosomes, the expression levels of RNA detected by the other methods were compared with that of ultracentrifugation. The Pearson's coefficient of correlation of exosomal RNA expression detected between the four isolation methods was determined. The hybrid method demonstrated the strongest correlation with ultracentrifugation (correlation coefficient 0.92), followed by precipitation and ultrafiltration with 0.88 and 0.87, respectively. The hybrid method was also highly correlated with precipitation and filtration, with correlation of 0.87 and 0.84 respectively. The correlation between precipitation and filtration was 0.84. The number of transcripts that are commonly detected by each method was also determined. A small number of transcripts were detected uniquely by ultracentrifugation, ultrafiltration, precipitation and hybrid method.

This exemplary novel approach of isolation of exosomes from cell culture medium or body fluids was systematically compared with current methods of isolation of exosomes. Yield and size distribution of exosomes isolated by the hybrid method are remarkably consistent with currently existing methods. Bioanalyzer profiles further indicated the consistency in RNA size distribution with existing methods. While other methods resulted in comparatively lower and inconsistent yield of exosomal RNA, the hybrid method consistently yields highest quantity of RNA from exosomes. The scalability of the hybrid approach has been scaled down to 25 mL volume of conditioned media, and could be readily scaled down to even lower volumes of media or body fluid. RNA-seq analysis further confirmed the hybrid method's ability to consistently and reproducibly isolate RNA transcripts from exosomes. Moreover, strong correlation of gene expression observed between the hybrid method and each of the other methods, including the ultracentrifugation method, underscored the reliability of its performance. Taken together, these results coupled with high and consistent yields of RNA, demonstrate the advantages of the hybrid method as the method of choice for isolation of exosomes for downstream exosomal RNA oriented/related studies.

### Example 41

### Cancer-Secreted EVs May Destroy the Barrier Function of Endothelial Monolayer

An in vitro permeability assay can be performed by measuring the traversing of rhodamine-labeled 70K dextran probes through cell monolayers growing on 0.4-μm filters. Treatment of the endothelial barrier with cancer cell EVs may cause passage of the fluorescent probes from top to the bottom wells in a manner that may be dependent on functional hY5 fragments. The trans-endothelial electrical resistance can be measured in cell monolayers, and treatment with cancer cell EVs may significantly reduce the unit area resistance compared to control EV treatment. The effect of treatment with EVs from cancer cells containing hY5 fragments on vascular destruction can be further tested in a 3D vascular sprouting assay. In this system, endothelial cells will form vascular sprouts after 4 to 5 days in culture. At that time, purified EVs from control or cancer cells can be added into the culture media and the effects on already established vascular structures analyzed 5 days later. Significant destruction of vascular structures may be observed with the treatment of hY5 fragment-containing EVs in comparison to the control. Lastly, to directly simulate the barrier-traversing step in metastasis, trans-endothelial invasion of cancer cells can be examined using cell monolayers grown on 3-μm filters. The number of GFP-labeled cancer cells that invade through the monolayer treated with cancer cell EVs may be significantly greater in comparison to the number that invade through untreated or control EV-treated cell monolayers. Pre-treatment of cancer cell EVs with an ASO that inhibits hY5 fragments may inhibit the number of GFP-labeled cancer cells that invade through the monolayer.

### Example 42

### Cancer Cell EVs May Induce Vascular Permeability and Promote Metastasis In Vivo

To further demonstrate the in vivo effect functional hY5 fragments in cancer cell EVs on endothelial barriers, EVs secreted by control cells, or cancer cells, can be injected into the tail vein of NOD/SCID/IL2Rγ-null (NSG) mice and examined lung and brain, organs that frequently host BC metastases, after EV treatment. The results may indicate that cancer cell EVs with functional hY5 fragments, but not EVs from control cells, can significantly increase hY5 fragment levels in lung and brain, and may be accompanied by enhanced vascular permeability. Alternatively, mice can be pretreated with EVs secreted by control or cancer cells before an intracardiac injection of luciferase-labeled cancer cells. Three weeks later, tissues can be collected for RT-qPCR of luciferase gene using mouse 18S as internal control to quantify metastases. Consistent with their effect on destroying the endothelial barriers, cancer cell EVs, but not control EVs, may significantly increase metastases in lung and brain. Pre-treatment of the cancer cell EVs with an ASO that inhibits hY5 fragments may inhibit the increase of metastases in lung and brain.

### Example 43

### hY5 Fragments in Cancer Cell EVs May Promote Tumor Invasion and Metastasis In Vivo

Cancer cell EVs containing functional hY5 fragments, hY5 fragments isolated from cancer cell EVs, or synthetic hY5 fragment polynucleotides can be contacted to an MCF-10A-derived tumorigenic line MCFDCIS, which forms comedo ductal carcinoma in situ-like lesions that spontaneously progress to invasive tumors. Compared to control EV treated cells, the treatment of MCFDCIS cells with the hY5 fragment preparations may show significantly enhanced migration in transwell and wound closure assays. Pre-treatment of the cancer cell EVs containing functional hY5 fragments, hY5 fragments isolated from cancer cell EVs, or synthetic hY5 fragment polynucleotides with an ASO that inhibits hY5 fragments may abolish the pro-migratory effect of the hY5 fragments.

Next orthotopic xenografts can be established using luciferase-labeled MCFDCIS cells with or without treatment with cancer cell EVs containing functional hY5 fragments, hY5 fragments isolated from cancer cell EVs, or synthetic hY5 fragment polynucleotides. Although hY5 fragment preparations may affect primary tumor growth, distant metastases may also be significantly induced in lung and brain in mice treated with the cancer cell EVs containing functional hY5 fragments, hY5 fragments isolated from cancer cell EVs, or synthetic hY5 fragment polynucleotides, compared to control EV treatments. Histological staining can be used to determine levels of local invasiveness. In vivo vascular permeability may be dramatically increased compared to the control group. A relatively high vascular permeability may be observed in the primary tumors of both groups. In mice treated with hY5 fragments, hY5 fragments would then be detected not only in primary tumors but also in the metastasis-free areas of distant organs. These results would indicate that EVs from tumor cells containing functional hY5 fragments have greater metastatic potential through the dual advantages of enhanced tumor cell invasion and weakened endothelial barriers in the host.

### Example 44

### ASO Treatment of Cancer Cells EVs can be Used to Suppress Metastasis and Restore Vascular Integrity In Vivo

To further explore the potential therapeutic effect of inhibiting functional hY5 fragments, xenografts can be established from highly-metastatic cancer cells that will be generated through explant culture of a spontaneous meningealmetastasis of cells. In vitro treatment of these cells with an ASO compound comprising the nucleotide sequence of 5′-CCC ACA AC-3′ (SEQ ID NO:7) may suppress migration, which would be consistent with the effect of hY5 fragments observed in other experiments. In vivo treatment with the ASO compound may reduce the volume of primary tumors and may suppress distant metastases to lung and brain compared to the groups that will receive EVs from control cells. Tumors treated with the ASO compound may have a clear margin with significantly reduced tumor cell infiltration into the surrounding tissues. The in vivo vascular permeability assay may indicate lack of rhodamine-dextran penetration into various tissues in tumor-free mice; conversely, leakage of the dye into these tissues in tumor-bearing animals may occur even at a premetastatic stage, which could suggest an effect of tumor-secreted factors in destroying the vascular integrity of a distant organ during early pre-metastatic site formation. Notably, treatment with the ASO compound may efficiently block this effect, and may restore the vascular integrity in tumor-bearing animals Thus, ASO compound treatment may be used to supress metastasis by reducing tumor invasiveness and restoring the barrier function of endothelial site cells.

### Example 45

### hY5 Fragments in Cancer Cell EVs May be Associated with Metastatic Cancer Progression

Because hY5 fragments that may have pro-metastatic and/or that may cause increased cancer cell progression are uniquely found in a functional form in EVs from cancer cells, it may be possible that cancer-secreted EVs containing functional hY5 fragments could be detected in the circulation of patients. Thus, functional hY5 fragments may serve as a prognostic marker for tumor progression potential or metastatic potential. As an example, the serum hY5 fragments levels can be measured in mice bearing xenograft tumors at either pre-metastatic (week 3 after cancer cell implantation) or metastatic stages (week 6 after cancer cell implantation) in comparison to tumor-free animals Circulating hY5 fragments that from cancer cell EVs and circulating EVs containing functional hY5 fragments that have pro-metastatic and/or that can cause increased cancer cell progression, may be significantly elevated in animals with tumors at both pre- and metastatic stages. Thus, hY5 fragments derived from primary tumor EVs with functional hY5 fragments and high metastatic potential may be detected in the blood at an early stage before clinical detection of metastasis.

To further determine if circulating hY5 fragments derived from EVs produced by primary tumors in cancer patients are functionally active in regulating endothelial cells, 3D vascular structures can be treated with serum from a healthy donor or a cancer patient with a high level of circulating hY5 fragments derived from EVs produced by primary tumors. The patient serum but not normal serum may result in destruction of vascular structures, which may be abolished by pre-treatment of the patient serum with the AMO compound. Using a logistic regression model, higher levels of circulating functional hY5 fragments may predict metastasis sensitively and specifically. In patients with paired serum and tumor specimens, a strong positive correlation may be detected between circulating and tumor hY5 fragments. Overall, the clinical data could indicate that hY5 fragments from cancer-cells may be used as a blood-based marker for the prediction or early diagnosis of cancer metastasis, and may play a role in promoting cancer progression.

### Example 46

### EV Electroporation

Cancer cell-derived EVs at a total protein concentration of 100 μg (measured by Bradford Assay) and 10 μg of an ASO inhibitor of a hY5 RNA fragment can be mixed in 400 μL of electroporation buffer (1.15 mM potassium phosphate pH 7.2, 25 mM potassium chloride, 21% Optiprep) and then electroporated in a 4 mm cuvette using a Gene Pulser Xcell Electorporation System (Biorad). After electroporation, the EVs can be tested for activity or functionality, or can be administered to a subject in need thereof.

1. A composition comprising an antisense masking oligonucleotide (AMO), wherein the AMO has anti-tumor activity, specifically binds to a RNA fragment of a primary RNA transcript of an extracellular cancer vesicle (ECV) and inhibits tumor progression mediated by the RNA fragment.

2. The composition of claim 1, wherein the RNA fragment is a human (h)Y fragment.

3. The composition of claim 2, wherein the human (h)Y fragment is hY5.

4. The composition of claim 1, wherein the RNA fragment is from about 8 to 40 nucleotides in length.

5. The composition of claim 4, wherein the RNA fragment is about 23, 29, or 31 nucleotides in length.

6. The composition of claim 1, wherein the RNA fragment comprises the sequence 5′ GUU GUG GG 3′ (SEQ ID NO: 1).

7. The composition of claim 1, wherein the ECV comprises at least one of: programmed cell death 6-interacting protein (PDCDIP), transferrin receptor (CD71), TSG101, or an Endosomal Sorting Complexes Required for Transport (ESCRT) protein complex.

8. The composition of claim 1, wherein the AMO comprises the sequence 5′-CCC ACA AC-3′ (SEQ ID NO: 7).

9. The composition of claim 1, wherein the AMO inhibits at least one of: apoptosis of non-tumor cells in a tumor microenvironment, angiogenesis in a tumor microenvironment, metastasis, inflammation or cell migration.

10. A method of treating cancer in a mammal comprising administering to the mammal, a composition or pharmaceutical composition of claim 1.

11. The method of claim 10, wherein the composition or pharmaceutical composition inhibits at least one of: apoptosis of non-tumor cells in a tumor microenvironment, angiogenesis in a tumor microenvironment, metastasis, inflammation or cell migration.

12. A method of producing a therapeutic ECV comprising an antisense masking oligonucleotide (AMO) that specifically binds to a RNA fragment of a primary RNA transcript of the ECV, wherein the RNA fragment mediates tumor progression, comprising:
(a) providing a cancer cell that can produce ECVs;
(b) allowing the cancer cell to produce the ECVs;
(c) transfecting an AMO in the ECVs; and
(d) isolating exosomes produced by the cell, wherein the ECVs comprise the AMO bound to the RNA fragment of a primary RNA transcript.

13. The method of claim 12, wherein the AMO inhibits at least one of: apoptosis of non-tumor cells in a tumor microenvironment, angiogenesis in a tumor microenvironment, metastasis, inflammation or cell migration.

14. A method of identifying an AMO that inhibits tumor progression mediated by a RNA fragment of a primary RNA transcript of an ECV, comprising:
(a) providing a testing system comprising ECVs and target cells, wherein the ECVs are located in proximity to the target cells;
(b) measuring tumor progression of the target cells; and
(c) identifying the AMO that inhibits tumor progression mediated by the RNA fragment of the primary RNA transcript of the ECVs.

15. The method of claim 14, wherein the system further comprises a cancer cell population that produces the ECVs.

16. The method of claim 14, wherein the RNA fragment is a human (h)Y fragment.

17. The method of claim 16, wherein the human (h)Y fragment is hY5.

18. The method of claim 14, wherein the RNA fragment is from about 8 to 40 nucleotides in length.

19. The method of claim 14, wherein the RNA fragment comprises the sequence 5′ GUU GUG GG 3′ (SEQ ID NO: 1).

20. The method of claim 14, wherein the AMO comprises the sequence 5′-CCC ACA AC-3′ (SEQ ID NO: 7).


SEQUENCE LISTING (46 sequences)

SEQ ID NO: 1 - RNA, 8 nt, Artificial Sequence
  Description of Artificial Sequence Synthetic oligonucleotide
guuguggg

SEQ ID NO: 2 - RNA, 31 nt, Artificial Sequence
  Description of Artificial Sequence Synthetic oligonucleotide
aguugguccgaguguuguggguuauuguuaa

SEQ ID NO: 3 - RNA, 23 nt, Artificial Sequence
  Description of Artificial Sequence Synthetic oligonucleotide
aguugguccgaguguuguggguu

SEQ ID NO: 4 - RNA, 23 nt, Artificial Sequence
  Description of Artificial Sequence Synthetic oligonucleotide
aguugguccgaguguuguggguu

SEQ ID NO: 5 - RNA, 31 nt, Artificial Sequence
  Description of Artificial Sequence Synthetic oligonucleotide
aguugguccgaguacguacaguuauuguuaa

SEQ ID NO: 6 - RNA, 83 nt, Artificial Sequence
  Description of Artificial Sequence Synthetic oligonucleotide
aguugguccgaguguuguggguuauuguuaaguugauuuaacauugucuccccccacaaccgcgcuugacuagcuugcuguuu

SEQ ID NO: 7 - DNA, 8 bp, Artificial Sequence
  Description of Artificial Sequence Synthetic oligonucleotide
cccacaac

SEQ ID NO: 8 - RNA, 32 nt, Artificial Sequence
  Description of Artificial Sequence Synthetic oligonucleotide
ccccacaaccgcgcuugacuagcuugcuguuu

SEQ ID NO: 9 - DNA, 32 bp, Artificial Sequence
  Description of Artificial Sequence Synthetic oligonucleotide
ccccacaaccgcgcttgactagcttgctgttt

SEQ ID NO: 10 - RNA, 23 nt, Artificial Sequence
  Description of Artificial Sequence Synthetic oligonucleotide
cccacaaccgcgcuugacuagcu

SEQ ID NO: 11 - DNA, 23 bp, Artificial Sequence
  Description of Artificial Sequence Synthetic oligonucleotide
cccacaaccgcgcttgactagct

SEQ ID NO: 12 - RNA, 24 nt, Artificial Sequence
  Description of Artificial Sequence Synthetic oligonucleotide
cccacaaccgcgcuuggacuagcu

SEQ ID NO: 13 - DNA, 24 bp, Artificial Sequence
  Description of Artificial Sequence Synthetic oligonucleotide
cccacaaccgcgcttggactagct

SEQ ID NO: 14 - RNA, 20 nt, Artificial Sequence
  Description of Artificial Sequence Synthetic oligonucleotide
cccacaacacuugacuagcu

SEQ ID NO: 15 - DNA, 20 bp, Artificial Sequence
  Description of Artificial Sequence Synthetic oligonucleotide
cccacaacacttgactagct

SEQ ID NO: 16 - RNA, 21 nt, Artificial Sequence
  Description of Artificial Sequence Synthetic oligonucleotide
cccacaacacuuggacuagcu

SEQ ID NO: 17 - DNA, 21 bp, Artificial Sequence
  Description of Artificial Sequence Synthetic oligonucleotide
cccacaacacttggactagct

SEQ ID NO: 18 - RNA, 23 nt, Artificial Sequence
  Description of Artificial Sequence Synthetic oligonucleotide
uuaacauugucuccccccacaac

SEQ ID NO: 19 - DNA, 23 bp, Artificial Sequence
  Description of Artificial Sequence Synthetic oligonucleotide
ttaacattgtctccccccacaac

SEQ ID NO: 20 - RNA, 18 nt, Artificial Sequence
  Description of Artificial Sequence Synthetic oligonucleotide
uuaacaauaacccacaac

SEQ ID NO: 21 - DNA, 18 bp, Artificial Sequence
  Description of Artificial Sequence Synthetic oligonucleotide
ttaacaataacccacaac

SEQ ID NO: 22 - DNA, 32 bp, Artificial Sequence; identical to SEQ ID NO: 9
  Description of Artificial Sequence Synthetic oligonucleotide

SEQ ID NO: 23 - DNA, 23 bp, Artificial Sequence
  Description of Artificial Sequence Synthetic oligonucleotide
cccacaaccgcgcttgactagct

SEQ ID NO: 24 - DNA, 24 bp, Artificial Sequence
  Description of Artificial Sequence Synthetic oligonucleotide
cccacaaccgcgcttggactagct

SEQ ID NO: 25 - DNA, 20 bp, Artificial Sequence
  Description of Artificial Sequence Synthetic oligonucleotide
cccacaacacttgactagct

SEQ ID NO: 26 - DNA, 21 bp, Artificial Sequence
  Description of Artificial Sequence Synthetic oligonucleotide
cccacaacacttggactagct

SEQ ID NO: 27 - DNA, 23 bp, Artificial Sequence
  Description of Artificial Sequence Synthetic oligonucleotide
ttaacattgtctccccccacaac

SEQ ID NO: 28 - DNA, 18 bp, Artificial Sequence
  Description of Artificial Sequence Synthetic oligonucleotide
ttaacaataacccacaac

SEQ ID NO: 29 - DNA, 8 bp, Artificial Sequence
  Description of Artificial Sequence Synthetic oligonucleotide
cccacaac

SEQ ID NO: 30 - RNA, 32 nt, Artificial Sequence; identical to SEQ ID NO: 8
  Description of Artificial Sequence Synthetic oligonucleotide

SEQ ID NO: 31 - RNA, 23 nt, Artificial Sequence
  Description of Artificial Sequence Synthetic oligonucleotide
cccacaaccgcgcuugacuagcu

SEQ ID NO: 32 - RNA, 24 nt, Artificial Sequence
  Description of Artificial Sequence Synthetic oligonucleotide
cccacaaccgcgcuuggacuagcu

SEQ ID NO: 33 - RNA, 20 nt, Artificial Sequence
  Description of Artificial Sequence Synthetic oligonucleotide
cccacaacacuugacuagcu

SEQ ID NO: 34 - RNA, 21 nt, Artificial Sequence
  Description of Artificial Sequence Synthetic oligonucleotide
cccacaacacuuggacuagcu

SEQ ID NO: 35 - RNA, 23 nt, Artificial Sequence
  Description of Artificial Sequence Synthetic oligonucleotide
uuaacauugucuccccccacaac

SEQ ID NO: 36 - RNA, 18 nt, Artificial Sequence
  Description of Artificial Sequence Synthetic oligonucleotide
uuaacaauaacccacaac

SEQ ID NO: 37 - DNA, 32 bp, Artificial Sequence
  Description of Artificial Sequence Synthetic oligonucleotide
cttaacaataacccacaacactcggaccaact

SEQ ID NO: 38 - RNA, 31 nt, Artificial Sequence; identical to SEQ ID NO: 2
  Description of Artificial Sequence Synthetic oligonucleotide

SEQ ID NO: 39 - RNA, 23 nt, Artificial Sequence
  Description of Artificial Sequence Synthetic oligonucleotide
aguugguccgaguguuguggguu

SEQ ID NO: 40 - RNA, 31 nt, Artificial Sequence
  Description of Artificial Sequence Synthetic oligonucleotide
uggugcguguuguuuagauuaagugguugac

SEQ ID NO: 41 - RNA, 23 nt, Artificial Sequence
  Description of Artificial Sequence Synthetic oligonucleotide
aguugguccgaguuuauuguuaa

SEQ ID NO: 42 - RNA, 31 nt, Artificial Sequence; identical to SEQ ID NO: 5
  Description of Artificial Sequence Synthetic oligonucleotide

SEQ ID NO: 43 - RNA, 32 nt, Artificial Sequence; identical to SEQ ID NO: 8
  Description of Artificial Sequence Synthetic oligonucleotide

SEQ ID NO: 44 - RNA, 83 nt, Artificial Sequence; identical to SEQ ID NO: 6
  Description of Artificial Sequence Synthetic oligonucleotide

SEQ ID NO: 45 - RNA, 32 nt, Artificial Sequence
  Description of Artificial Sequence Synthetic oligonucleotide
aguugguccgaguguuguggguuauuguuaag

SEQ ID NO: 46 - RNA, 32 nt, Artificial Sequence; identical to SEQ ID NO: 8
  Description of Artificial Sequence Synthetic oligonucleotide